(19) United States
(12) Patent Application Publication
(10) Pub. No.: US 2023/0321278 A1
(43) Pub. Date: Oct. 12, 2023

(54) AAV VECTOR DELIVERY SYSTEMS

(71) Applicant: President and Fellows of Harvard College, Cambridge, MA (US)

(72) Inventors: Ya-Chieh Hsu, Cambridge, MA (US); Yulia Shwartz, Cambridge, MA (US); Meryem Gonzalez Celeiro, Cambridge, MA (US); Emily Rivkah Scott-Solomon, Cambridge, MA (US)

(21) Appl. No.: 18/015,521

(22) PCT Filed: Jul. 12, 2021
(86) PCT No.: PCT/US2021/041336

§ 371 Date: Jan. 10, 2023

Related U.S. Application Data
(60) Provisional application No. 63/050,401, filed on Jul. 10, 2020.

(51) Int. Cl. A61K 48/00; C12N 15/86; C12N 9/88; A61K 38/18; A61P 17/14

(52) U.S. Cl. CPC A61K 48/0058; C12N 15/86; C12N 9/88; C12Y 401/02042; A61K 48/0041; A61K 38/1825; A61P 17/14; C12N 2830/008; C12N 2750/14143; C12N 2750/14145

(57) ABSTRACT

Disclosed herein are viral vector delivery systems and methods for the targeted delivery of genes to a predetermined skin cell. The viral vector delivery systems may be adeno-associated viral (AAV) vector delivery systems.

### RELATED APPLICATION(S)

This application is related to and claims the benefit of U.S. Provisional Application No. 63/050,401, filed Jul. 10, 2020. The entire teachings of the application are incorporated herein by reference.

### BACKGROUND OF THE INVENTION

Skin diseases are diverse, complex, and affect a large population—almost every household has someone who is affected by a skin disease or condition. For example, 50% of males by the age 50 and 40% of females by menopause have some degree of androgenic alopecia (male pattern baldness). In addition, at least 6 million people suffer from chronic wounds within the United States alone. For example, epidermolysis bullosa (butterfly children), a group of genetic diseases that cause skin to blister easily, remains one of the most devastating genetic disorders.

Over the past decades, progress in stem cell biology and skin biology have identified several genes and pathways that might serve as potential therapeutic targets. However, a major roadblock to translating these findings is the lack of a safe and effective method to deliver modifying agents of these pathways locally to the desired skin cells—agonists or antagonists of a gene may not always be available, and a lack of safe technologies to deliver DNA only into the desired tissues and cell types remains one of the greatest challenges.

### SUMMARY OF THE INVENTION

Adeno-associated virus (AAV) is one of the most actively investigated gene therapy vehicles. Compared to other viruses or delivery methods, AAV is among the safest—it elicits mild immune responses, infects dividing and quiescent cells, is replication-defective, and does not integrate into the genome. However, one key challenge is to deliver AAV only into desired tissues or cell types, since systemic delivery of almost all AAVs has a strong tropism for the liver.

To investigate the potential of using AAVs to treat skin diseases, several different viral delivery methods have been tested and compared. It was shown that local intradermal injection of AAV8 infected a wide variety of cell types in skin, including various dermal fibroblasts, dermal papilla (a cluster of dermal cells critical in secreting factors to promote hair follicle regeneration), Schwann cells, and dermal adipocytes. Under the appropriate titer, only the injected area is infected, not the surrounding skin or elsewhere in the body. These results demonstrate the great promise of using AAVs in treating a wide variety of skin diseases.

Disclosed herein are delivery systems. The delivery systems (e.g., viral vector delivery systems) include an adeno-associated virus (AAV) and a promoter for delivery of a gene to a cell selected from the group consisting of fibroblasts, dermal papilla, adipocytes, arrector pili muscle, sensory nerves, sympathetic nerves, immune cells, and panniculus carnosus.

In some embodiments, the promoter is selected from the group consisting of CAG, EF1a, NPY, and hSYN. In some embodiments, the AAV is selected from the group consisting of AAV2, AAV6, AAV8, AAV9, AAVrh10, AAV-DJ, AAV-PHP.S, and AAV-retro, and in more particular aspects, the AAV is selected from the group consisting of AAV8, AAVrh10, AAV6, AAV-PHP.S, and AAV-retro.

In one embodiment, the AAV comprises AAV2, the promoter comprises CAG, and the cell comprises adipocytes. In one embodiment, the AAV comprises AAV9, the promoter comprises CAG, and the cell is selected from the group consisting of adipocytes, fibroblasts, and arrector pili muscle. In one embodiment, the AAV comprises AAV-DJ, the promoter comprises CAG, and the cell comprises adipocytes. In one embodiment, the AAV comprises AAV8, the promoter comprises CAG, and the cell is selected from the group consisting of fibroblasts, dermal papilla, adipocytes, arrector pili muscle, and immune cells. In one embodiment, the AAV comprises AAV8, the promoter comprises EF1a, and the cell is selected from the group consisting of fibroblasts, dermal papilla, adipocytes, arrector pili muscle, and immune cells. In one embodiment, the AAV comprises AAVrh10, the promoter comprises CAG, and the cell is selected from the group consisting of fibroblasts, adipocytes, and arrector pili muscle. In one embodiment, the AAV comprises AAV6, the promoter comprises CAG, and the cell is selected from the group consisting of fibroblasts, adipocytes, and arrector pili muscle. In one embodiment, the AAV comprises AAV6, the promoter comprises EF1a, and the cell comprises adipocytes and arrector pili muscle. In one embodiment, the AAV comprises AAV-PHP.S, the promoter comprises CAG, and the cell is selected from the group consisting of fibroblasts, adipocytes, arrector pili muscle, sensory nerves, sympathetic nerves and panniculus carnosus. In one embodiment, the AAV comprises AAV-PHP.S, the promoter comprises EF1a, and the cell is selected from the group consisting of fibroblasts, dermal papilla, adipocytes, and arrector pili muscle. In one embodiment, the AAV comprises AAV-PHP.S, the promoter comprises NPY, and the cell is selected from the group consisting of sensory nerves and sympathetic nerves. In one embodiment, the AAV comprises AAV-PHP.S, the promoter comprises hSYN, and the cell is selected from the group consisting of sensory nerves and sympathetic nerves. In one embodiment, the AAV comprises AAV-retro, the promoter comprises CAG, and the cell is selected from the group consisting of adipocytes and sympathetic nerves. In one embodiment, the AAV comprises AAV-retro, the promoter comprises hSYN, and the cell comprises sympathetic nerves.

Also disclosed herein are delivery systems comprising an adeno-associated virus (AAV) and a promoter for delivery of a gene to an arrector pili muscle (APM) or a fibroblast. In some embodiments, the AAV is AAV-PHP.S. In some embodiments, the promoter is CAG.

Also disclosed herein are delivery systems comprising an adeno-associated virus (AAV) and a promoter for delivery of a gene to a skin cell. In some embodiments, the AAV is AAV-PHP.S, the enhancer is CAG, and the skin cell is not a sympathetic nerve, a blood vessel, or a dermal sheath. In some embodiments, the gene is a DTA.

Disclosed herein are delivery systems comprising an adeno-associated virus (AAV) and a promoter for delivery of a gene to a hair follicle stem cell (HFSC). In some embodiments, the AAV is AAV8, the promoter is CAG, and/or the gene is FGF18.

In some embodiments, the delivery system is suitable for administration to a patient via intradermal injection.

Also disclosed herein are pharmaceutical compositions comprising a delivery system disclosed herein.

Also disclosed herein are methods of treating a condition, disease, or disorder in a subject comprising administering a pharmaceutical composition described herein to the subject.

Also disclosed herein are methods of encouraging hair growth in a subject. The methods include elevating sympathetic nerve activity by exposing the subject to a cold temperature for a period of at least two hours.

In some embodiments, the exposure to the cold temperature activates hair follicle stem cells (HFSCs). In some embodiments, the exposure to the cold temperature results in enhanced c-Fos expression. In one embodiment, the cold temperature is a temperature of about 5° C. In some embodiments, the cold temperature is applied directly and/or specifically to the location of desired hair growth. In one embodiment, the location of desired hair growth is the scalp.

Unless defined otherwise, all technical and scientific terms used herein have the meaning commonly understood by a person skilled in the art. The following references provide one of skill with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3.sup.rd ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well-known and commonly used in the art. Standard techniques may be used for chemical synthesis, chemical analyses, pharmaceutical preparation, formulation, and delivery and treatment of patients.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E demonstrate sympathectomy delays anagen entry whereas elevation of sympathetic tone drives anagen entry. FIG. 1A shows immunofluorescent staining for tyrosine hydroxylase (TH) in control and sympathectomized (6-OHDA) skin. FIG. 1B shows immuno-colocalization of EdU, CD34, and P-Cadherin (PCAD) in control and sympathectomized P25 skin. FIG. 1C shows Hematoxylin & Eosin (H&E) staining of control and sympathectomized skin. Graph: hair cycle distribution at P30 (n=4-5 mice per condition, 20 hair follicles (HF) per mouse). FIG. 1D shows H&E staining of control and sympathectomized TH-CreER; Rosa-lsl-attenuated DTA (TH-CreER; DTA) skin. Graph: hair cycle distribution at P31 - P34 (n=4-5 mice per condition, 10 HF per mouse). FIG. 1E shows topical application of isoproterenol at the 2nd telogen results in precocious anagen entry (n=10 mice per condition). Graph: back skin hair regrowth (%). Unless otherwise specified, all scale bars=50 μm. Data are mean±SEM. *: p<0.05; **: p<0.01; ***: p<0.001. See also FIG. 8.

FIGS. 2A-2F demonstrate HFSC activity is modulated by ADRB2. FIG. 2A provides expression of adrenergic receptors in HFSCs (RNA-seq). FIG. 2B shows chromatin modifications around the loci of Adrb genes in HFSCs. FIG. 2C provides a schematic of K15-CrePGR activity (blue) and experimental design (arrow denotes harvesting). qRT-PCR of Adrb2 from FACS-purified HFSCs of control and K15-CrePGR; Adrb2 fl/fl (Adrb2-cKO) mice (n=3 mice per condition). FIG. 2D shows H&E staining of control and Adrb2-cKO skin. Graph: hair cycle distribution (n=5-7 mice per condition, 20 HF per mouse). FIG. 2E shows topical application of procaterol (ADRB2 agonist) drives premature anagen entry (n=10 mice per condition). Graph: back skin hair regrowth (%). FIG. 2F shows colony formation assay on control and procaterol-treated human HFSCs. Graph: area covered by colonies (n=3-5 wells per condition). Data are mean±SEM. *: p<0.05; **: p<0.01; ***: p<0.001. See also FIG. 9.

FIGS. 3A-3J demonstrate transcriptome analyses of Adrb2-depleted HFSCs. FIG. 3A provides a schematic of workflow. FIG. 3B shows immunofluorescent staining for phospho-histone H3 (pH3), CD34, and PCAD in control and Adrb2-cKO mice. FIG. 3C provides principal component analysis (PCA) comparing the transcriptome of control and Adrb2-cKO HFSCs. FIG. 3D provides Ingenuity Pathway Analysis (IPA) of significantly deregulated genes in Adrb2-cKO mice. FIG. 3E shows heatmap plotting expression of cell cycle-related genes. Positive Z-score depicts higher expression; negative Z-score indicates lower expression. FIG. 3F shows quiescent-related transcription factors in control and Adrb2-cKO HFSCs. High-low bar graph, line at mean. FIG. 3G shows qRT-PCR of Foxpl and Fgf18 from FACS-purified HFSCs (n=2-3 mice per condition). FIG. 3H provides a schematic of bulge and sympathetic innervation (HFSCs are the outer bulge and K6+ cells are the inner bulge. Sympathetic nerve innervates only HFSCs). FIG. 31 shows in situ hybridization of Fgf18 in control and Adrb2-cKO mice (arrowheads: positive signals in HFSCs). Graph: Fgf18+ signal spots in HFSCs. FIG. 3J shows H&E staining of control and AAV8-CAG-FGF18-3XHA (AAV-FGF18) injected mice. Graph: hair cycle distribution (n=5 mice per condition, 10 HF per mouse). Scale bar, 25 μm in FIG. 31. Data are mean±SEM. *: p<0.05; **: p<0.01; ***: p<0.001; n.s.: not significant. See also FIG. 10.

FIGS. 4A-4G demonstrate that a sympathetic network surrounds HFSCs and forms synapse-like connections with HFSCs. FIG. 4A shows immunofluorescent staining for TH and PCAD reveals a sympathetic network. Insert: nerve bridges (arrowhead) between the main bundles. FIG. 4B shows immunofluorescent staining for TH, Smooth muscle actin (SMA), and PCAD. Sympathetic nerve fibers (arrowheads) extend beyond APMs and approach HFSCs at both the old and new bulge. FIG. 4C shows a main sympathetic bundle innervates the APM and the old bulge (caudal side), while smaller branches from both the caudal and rostral bundles innervate the new bulge and hair germ. A bottom view of the 3D-reconstructed image in FIG. 4C is seen in C′. A single orthogonal section showing points of contact (arrowheads) between HFSCs and sympathetic fibers is shown is C″. Schematic: wrapping of sympathetic nerves (green) around the old bulge (light pink), new bulge (light blue), and hair germ (light blue). Eye cartoon: viewing angle in C′. Dashed line: plane of orthogonal view in C″. FIG. 4D shows sympathetic nerve fibers colocalize with the pre-synaptic marker Synaptotagmin when approaching HFSCs (arrowheads in insert: points of nerve-HFSC interaction). FIG. 4E shows immunofluorescent staining for TH and PCAD shows varicose axons (arrowheads in insert: varicosities). FIG. 4F provides a schematic: synapse-like connections between HFSCs and sympathetic nerves. 3D electron microscope (EM) reconstruction of sympathetic axon terminals demonstrates varicose regions (red arrows). Right: Tracing of the same two axons (axon 1 and axon 2) shows changes in axon diameter and Schwann cell wrapping (Sch, pink). Plane a, varicose region (black arrow: exposed axon). Plane b, non-varicose region. FIG. 4G shows 3D-reconstruction of EM stacks showing sympathetic (SN) axons (green), HFSCs (blue), and endoneurial fibroblast-like cells (EFLC, brown, component of endoneurium). Insert shows that endoneurium opens up on the side facing HFSCs to expose enwrapped axons. Right: Single EM sections showing that the endoneurium is closed when sympathetic axons are farther away from HFSCs, but becomes open when the axons approach HFSCs. Scale bar, 10 μm in inserts FIG. 4D and FIG. 4E; 1 μm in FIG. 4F and FIG. 4G. See also FIG. 11.

FIGS. 5A-5G demonstrate APMs provide stable anchors that maintain sympathetic innervations to HFSCs. FIG. 5A provides a schematic: SMA-YFP-DTR construct and expression patterns (green). FIG. 5B shows co-localization of YFP and ITGA8 in diphtheria toxin (DT) injected control and SMA-YFP-DTR mice. FIGS. 5C-5D show TH and ITGA8 immunofluorescent staining (in FIG. 5C) and TH and PCAD immunofluorescent staining (in FIG. 5D) in DT injected control and SMA-YFP-DTR mice (n=3 mice per condition). Arrowheads: APMs in FIG. 5C and points of nerve-HFSC interaction in FIG. 5D. Loss of APMs leads to loss of sympathetic innervations to HFSCs. FIG. 5E shows H&E staining in DT injected control and SMA-YFP-DTR showing a delay in anagen entry of APM ablated mice (n=3 mice per condition). FIG. 5F provides a schematic: experimental design. APMs are the only cells that carry both Myh11-CreER and AAV-PHP.S-flex-DTA. Immunofluorescent staining for TH and ITGA8 in Myh11-CreER mice injected with AAV-PHP.S-flex-DTA (control: treated with EtOH; Myh11-AAV-DTA: treated with 4-OH-tamoxifen) shows the absence of HFSC innervation in APM ablated mice (n=4 mice per condition). FIG. 5G provides a schematic: Myh11-CreER activity (green) and experimental design (arrows: harvesting). Immunofluorescence and quantification of ITGA8 and YFP colocalization in Myh11-CreER; Rosa-lsl-YFP mice (n=3 mice, 7-12 APMs per mouse). Tam, tamoxifen or 4-OH-tamoxifen; Telo, telogen; Ana, anagen. See also FIG. 12.

FIGS. 6A-6F demonstrate that cold temperature causes piloerection and HFSC activation. FIG. 6A provides a schematic showing sympathetic axons extend to HFSCs while cell bodies are at the sympathetic ganglia. FIG. 6B shows immunofluorescent staining of TH and c-FOS in the sympathetic ganglia from mice under thermoneutral (control) or cold exposure for 2 hours. Graph: % of c-FOS positive cells per ganglion (n=2 mice per condition, 3-5 ganglia per animal). FIG. 6C shows norepinephrine concentration in the skin after 2 hours of cold exposure (n=6 mice per condition). FIG. 6D shows cold exposure results in piloerection (goosebumps). Magnification of the boxed area shows erection of the hair. FIG. 6E provides a schematic: experimental design (arrow: harvesting). 2 weeks of cold exposure in 2nd telogen results in premature anagen entry (n=9 mice per condition). Graph: % of hair regrowth in back skin. FIG. 6F shows H&E staining of control and 2-week cold exposed skin. Data are mean±SEM. *: p<0.05; **: p<0.01; ***: p<0.001.

FIGS. 7A-7J demonstrate SHH regulates APM development and sympathetic innervation to HFSCs. FIG. 7A provides a schematic: sequential development of hair follicles, APMs, and sympathetic innervations. FIG. 7B shows immunofluorescent staining of ITGA8 and TH. Arrowheads: APMs; solid circles: dermal papilla. FIG. 7C shows LacZ and ITGA8 co-localization at P2 Glil-LacZ skin. FIG. 7D shows ITGA8, H&E, and Masson trichrome staining of control and Pdgfra-Cre; Smo fl/fl (Smo-cKO) mice at P4. Graph: % of HFs with APMs (n=3 mice per condition, 200-280 HF per mouse). FIG. 7E shows ITGA8 and TH immunofluorescent staining on control and Smo-cKO mice at P8. FIG. 7F shows Keratin 14 (K14) and ITGA8 immunofluorescent staining of control and K14-Cre; Shh fl/fl on PO skin. FIG. 7G shows K14 and ITGA8 immunofluorescent staining of control and K14-Cre; Rosa-lsl-rtTA; TetO-P27 (K14-P27) mice on P4 skin. Graph: % of HFs with APMs (n=2 mice per condition, 120-180 HF per mouse). FIG. 7H shows in situ hybridization of Shh in control and K14-P27 mice at P4. FIGS. 7I-7J provide immunofluorescent staining of nephronectin (NPNT) in control, K14-Cre; Shh fl/fl (FIG. 7I) and Smo-cKO (FIG. 7J) mice. Data are mean±SEM. *: p<0.05; **: p<0.01; ***: p<0.001. n.s.: not significant. See also FIGS. 13 and 14.

FIGS. 8A-8G demonstrate sympathectomy does not result in overt changes of other skin cell types (related to FIG. 1). FIG. 8A provides a schematic of the tri-lineage unit. FIG. 8B shows immunofluorescent staining for active Caspase3 (aCAS3), CD34, and PCAD in control and sympathectomized (6-OHDA) mice. FIGS. 8C-8D provide intact sensory innervation (TUJ1) of the hair follicles (FIG. 8C, above the bulge) and Merkel cells (FIG. 8D, marked by K8) in sympathectomized mice. FIGS. 8E-8F provide integrin alpha8 (ITGA8) staining shows intact APMs in both models of sympathectomized mice: 6-OHDA and TH-CreER; Rosa-lsl-attenuated DTA (TH-CreER; DTA). FIG. 8G shows intact sensory innervation (TUJ1) of the hair follicle in TH-CreER; DTA mice.

FIGS. 9A-9J demonstrate nerve-derived norepinephrine impacts HFSC function whereas adrenal gland-derived catecholamines are dispensable (related to FIG. 2). FIG. 9A shows norepinephrine content in the skin of control and sympathectomized mice 7 days after injection (n=6 mice per condition). FIG. 9B shows chromatin modifications around the loci of Adra gene family. FIG. 9C shows K15-CrePGR; Adrb2 fl/fl (Adrb2-cKO) mice exhibit a delay in anagen entry. FIGS. 9D-9E provide immunofluorescent staining for TH (FIG. 9D) and aCAS3 (FIG. 9E) in control and Adrb2-cKO mice. FIG. 9F provides a schematic of adrenalectomy (ADX) experimental design. CORT: corticosterone supplement. Arrow: harvesting. FIGS. 9G-9I provide hormone concentrations in the plasma of mice that underwent a sham operation (sham) and ADX mice supplemented with corticosterone (ADX+CORT). FIG. 9J shows H&E staining of sham-operated and ADX+CORT mice. Graph: hair cycle distribution (n=4-6 mice per condition, 15 HF per mouse). Data are mean±SEM. *: p<0.05; **: p<0.01; ***: p<0.001; n.s.: not significant.

FIGS. 10A-10H demonstrate validation of hair cycle stage, gene expression, and complementary pathway analysis of control and Adrb2 depleted HFSCs (related to FIG. 3). FIG. 10A shows H&E staining of control and Adrb2-cKO skin collected for RNA-seq. FIG. 10B provide a heatmap of differentially expressed genes in control and Adrb2-cKO HFSCs. p_{adj}<0.1 and absolute fold change≥2. FIG. 10C shows Adrb2 normalized counts on sorted HFSCs of control and Adrb2-cKO showing efficient knockout. High-low bar graph, line at mean. FIG. 10D provides a Gene Ontology analysis of biological processes of significantly deregulated genes in Adrb2-cKO HFSCs. FIGS. 10E-10F provide heatmaps plotting expression of genes involved in oxidative phosphorylation (FIG. 10E) and ribosomal machinery (FIG. 10F). Positive Z-score depicts higher expression, negative Z-score indicates lower expression. FIG. 10G provides a schematic illustrating how sympathetic nerve dependent Fgf18 levels are propagated beyond innervation site to affect all HFSCs. FIG. 10H shows intact APM (ITGA8) and sympathetic innervation (TH) in AAV8-CAG-FGF18-3XHA (AAV-FGF18) injected mice (indicated by arrowhead). Immunofluorescent staining for HA indicates efficient infection (shown in insert). Scale bar, 50 μm. *: p<0.05.

FIGS. 11A-11I demonstrate EM and immunofluorescent analysis reveal synapse-like structures (related to FIG. 4). FIG. 11A shows immunofluorescent staining of TH and PCAD illustrating the complexity of the sympathetic network. FIG. 11B provides a graph: sympathetic innervation frequency at different positions (n=2-3, 10-30 HF per mouse). FIG. 11C shows interactions between sympathetic nerve and HFSCs located at different positions. All panels show a main sympathetic bundle innervating APM. In addition, smaller nerve branches innervate the new bulge and hair germ. Innervations (arrowheads) can diverge from a caudal nerve bundle (left panel) or from a rostral bundle (right panel). Top or bottom view of the corresponding 3D reconstructed hair follicle are provided in C′. Single orthogonal sections demonstrating proximity and points of contact (arrowheads) between HFSCs and sympathetic fibers are provided in C″. Dashed lines: plane of orthogonal view in C″. Eye cartoon: viewing angles. FIGS. 11D-11E show sympathetic nerve fibers colocalize with the pre-synaptic markers Synaptophysin (FIG. 11D) and vesicular monoamine transporter 2 (VMAT2) (FIG. 11E) when approaching HFSCs (arrowheads: points of nerve-HFSC interaction). FIG. 11F provides a schematic of synapse-like structures between sympathetic nerves and HFSCs. FIG. 11G shows single plane images (by serial block face scanning EM) of several independent hair follicles showing exposed axons (arrowheads). FIG. 11H shows EM showing neurotransmitter vesicles in sympathetic nerves near HFSCs (white arrowheads). Black arrowheads: exposed axons. FIG. 111 shows sensory axons (yellow) and terminal Schwann cells (pink) exhibit different morphology than sympathetic axons. Scale bar, 10 μm in inserts in FIG. 11D and FIG. 11E; 1 μm in FIG. 11G and FIG. 11H; 100 nm in inserts in FIG. 11H.

FIGS. 12A-12K demonstrate an extended analysis of SMA-YFP-DTR model and AAV-PHP.S cellular tropism in the skin (related to FIG. 5). FIG. 12A provides maximum intensity projection images of immunofluorescent staining for aCAS3, TH, and PCAD in control and K15-CrePGR; Rosa-lsl-DTA (K15-DTA) mice (n=2-3 mice per condition). FIGS. 12B-12C show immunofluorescent staining for aCAS3 (FIG. 12B) and endothelial marker CD31 (FIG. 12C) in DT injected control and SMA-YFP-DTR mice 2 days after injection. FIG. 12D shows immunofluorescent staining for PCAD, SMA, and CD31 in control and DT injected SMA-YFP-DTR mice 14 days after injection. Inserts show both original images (top) as well as reconstructed volume (vol) of CD31 and SMA+ staining in the blood vessel (bottom). Graph: volume of SMA+ positive cells adjacent to endothelial cells as % of the total volume of endothelial cells (n=3 mice per condition). FIG. 12E shows immunofluorescent staining of CD140a and SMA in control and SMA-YFP-DTR mice showing intact dermal sheath in APM ablated mice (n=3 mice per condition, 3-8 images per mouse). FIG. 12F shows immunofluorescent staining of tdTomato (TOM) in mice intradermally (id) injected with AAV-PHP.S-CAG-tdTomato (n=3 mice). Schematic summarizes infected cell types. FIGS. 12G-12J show immunofluorescent staining shows that AAV-PHP.S-CAG-tdTomato infects APMs (ITGA8 in FIG. 12G) but not sympathetic nerve (TH in FIG. 12H), blood vessels (both CD31 endothelial cells and SMA positive cells in FIG. 12I), or dermal sheath (CD140a in FIG. 12J) (n=3 mice). FIG. 12K shows immunofluorescent staining of ITGA8 and YFP on untreated Myh11 -CreER; Rosa-lsl-YFP (Myh11-CreER; YFP) mice showing minimum leakiness of Myh11-CreER without tamoxifen induction. Scale bar, 10 μm in FIG. 12A. n.s.: not significant.

FIGS. 13A-13G demonstrate hair follicle differentiation when Smo is depleted from dermal fibroblasts (related to FIG. 7). FIG. 13A shows co-localization of ITGA8 and YFP in Pdgfra-Cre; Rosa-lsl-YFP (Pdgfra-Cre; YFP) skin. FIG. 13B shows immunofluorescent staining of TH and YFP in Pdgfra-Cre; Rosa-lsl-YFP skin. FIG. 13C shows Shh in situ hybridization in control and Pdgfra-Cre; Smo fl/fl (Smo-cKO) mice at P4. FIGS. 13D-13E shows immunofluorescent staining of APMs (SMA or ITGA8) and hair follicles (PCAD or SOX9) in control and Smo-cKO mice at P8. FIGS. 13F-13G show immunofluorescent staining for differentiation markers Keratin 82 (K82), GATA3, and Keratin 6 (K6) in control and Smo-cKO P8 skin. Scale bar, 10 μm in inserts FIG. 13F and FIG. 13G.

FIGS. 14A-14G demonstrate hair follicle-derived SHH signaling is essential for APM 25 development (related to FIG. 7). FIG. 14A shows qRT-PCR for Shh, Dhh, and Ihh from PO skin (n=3). FIG. 14B shows ITGA8 and TH immunofluorescent staining in control and Dhh knockout mice (Dhh −/−) at P5. Graph: Percent of HFs with APMs (n=2-3 mice per condition, 82-211 HF per mouse). FIG. 14C shows ITGA8 and TH immunofluorescent staining in control and Advillin-Cre; Shh fl/fl (Avil-Cre Shh fl/fl) at P4. Graph: Percent of HFs with APMs (n=2 mice per condition, 150-219 HF per mouse). FIG. 14D shows immunofluorescent staining of pH3 and K14 in control and K14-Cre; Rosa-lsl-rtTA; TetO-P27 (K14-P27) mice at P4. Graph: number of proliferating HF cells (n=3 mice per condition, 15-75 HF per mouse). FIG. 14E shows immunofluorescent staining for differentiation markers PCAD, GATA3, K82, and K6 in control and K14-P27 mice at P4. FIGS. 14F-14G provide a model summarizing the formation and function of the tri-lineage unit during development, tissue maintenance, and upon cold stimulation. Scale bar, 10 μm in inserts in FIG. 14E. Data are mean±SEM. *: p<0.05; **: p<0.01; ***: p<0.001. n.s.: not significant.

FIGS. 15A-15D demonstrate AAV8/6/rh10 serotype efficiently infects dermal fibroblasts. Intra dermal (local) injection of 5E10 genome content of different AAV serotypes carrying fluorescent reporter (GFP/tdTOMATO) under the control of CAG promoter. FIGS. 15A-15D show sections through the back skin (1 week after infection) showing different AAV serotypes infection pattern (cellular tropism). AAV is depicted in white.

FIGS. 16A-16E demonstrate that all AAV serotypes infect adipocytes. FIGS. 16A-16E provide immunofluorescent staining for GFP/tdTOMATO (green) and adipocyte marker PLIN (red). Sections through the back skin (1 week after infection) following intradermal injection of 5E10 genome content of different AAV serotypes carrying fluorescent reporter (GFP/tdTOMATO) under the control of CAG promoter. Colocalization between AAV and PLIN shows that all serotypes tested can infect adipocytes with different efficiency (AAV-retro is on the lower scale infecting approximately 30-40% of the adipocytes).

FIGS. 17A-17E demonstrate AAV-PHP.S-CAG efficiently infect arrector pili muscles. FIGS. 17A-17E show immunofluorescent staining for GFP/tdTOMATO (green) and arrector pili muscle ITGA8 (red). Sections through the back skin (1-3 weeks after infection) following intradermal injection of 5E10 genome content of different AAV serotypes carrying fluorescent reporter (GFP/tdTOMATO) under the control of CAG promoter. Colocalization between AAV and ITGA8 shows that AAV-PHP.S can be used to efficiently target arrector pili muscle (FIG. 17E).

FIGS. 18A-18E demonstrate AAV-PHP.S efficiently infect sensory and sympathetic nerves and AAV retro efficiently infect sympathetic nerves. FIGS. 18A-18B shows systemic administration (tail vein) of E12 genome content AAV-PHP.S caring fluorescent reporter under CAG promoter efficiently infects sensory and sympathetic nerves. FIG. 18A top panel shows section through the back skin depicting AAV (green) infection pattern. FIG. 18A lower panel shows colocalization between AAV (green) and pan neuronal marker TUJ1 (red) in the back skin showing efficient infection of hair follicle sensory innervation (left) and Interfollicular epidermal innervation (right). FIG. 18B shows colocalization between AAV (green) and Sympathetic nerve marker TH (Tyrosine hydroxylase) show efficient infection of sympathetic nerves in the back skin. FIG. 18C shows intradermal administration of 2E10 genome content of two AAV-PHP.S caring fluorescent reporter under the control of human Synapsin promoter (red) and NPY (green) in newborn pups show efficient labeling of subsets of sympathetic nerves in the sympathetic ganglia 3 weeks post infection. FIG. 18D (left panel) shows intradermal administration of 5E10 genome content of AAV-retro serotype caring fluorescent reporter under the control of CAG promoter (green), leads to efficient labeling of sympathetic ganglia (left panel) and sympathetic nerves in the back skin (right panel) 1 week after infection. In addition, some adipocytes are infected. FIG. 18E shows intradermal administration of 5E10 genome content of AAV-retro serotype caring fluorescent reporter under the control of hSYN promoter show specific labeling of sympathetic nerves.

FIGS. 19A-19C demonstrate that using different ubiquitous promoters affects cellular tropism. FIGS. 19A-19C provide comparison between EF1a and CAG promoters driving the expression of fluorescent reporter. FIG. 19A shows when AAV8 is used there is no significant difference in the infected fibroblasts populations within the skin, as demonstrated by immunofluorescent staining (left). Nevertheless, EF1a promoter is less efficient in infecting adipocytes compared to CAG. FIGS. 19B-19C show that when packaged in AAV-PHP.S, EF1a showed significant increase in dermal papilla (arrow heads in FIG. 19B) infection and significant decrease in arrector pili muscle infection (FIG. 19C) compared to CAG. FIG. 19C shows colocalization between AAV (green) and arrector pili muscle marker ITGA8 showing that CAG promoter is significantly more efficient in infecting arrector pili muscle compared to EF1a promoter.

FIGS. 20A-20C demonstrate functional application of AAV. FIGS. 20A-20B show intradermal injection of 4-5E10 genome content of AAV8 carrying known factors that affect hair cycle. FIG. 20A shows injection of AAV8 caring FGF18 (known factor that promotes hair follicle stem cells quiescence) under CAG promoter inhibits anagen entry as seen from the skin pictures on the left and hematoxylin and eosin staining on the right. Graph depicts hair cycle distribution showing significant delay in anagen entry following AAV mediated FGF18 delivery. FIG. 20B shows injection of AAV8 caring SHH (known factor that promotes hair follicle stem cells proliferation) under CAG promoter promotes anagen entry as seen from the skin pictures. FIG. 20C shows pipetting (not invasive procedure) of 3E10 genome content of AAV8 caring fluorescent reporter (green) under CAG promoter into full thickness 6 mm wound leads to efficient infection of cells within the skin.

FIGS. 21A-21D demonstrate capsid serotypes influence the transduction pattern of AAV. FIG. 21A provides a schematic of a workflow chart. FIG. 21B provides a schematic of AAV intradermal injection in skin. HFSC; blue, arrector pili muscle; red, and adipocytes; yellow. FIG. 21C provides an experimental scheme for AAV injection in adult mice. FIG. 21D shows immunofluorescent staining of GFP and different markers for skin cell types. Perilipin for adipocyte; ItgA8 for arrector pili muscle (APM); CD140a for dermal fibroblast.

FIGS. 22A-22D demonstrate that PO injection showed robust long-lasting AAV transduction. FIG. 22A provides an experimental scheme for PO injection, harvest, and observation. FIG. 22B provides immunofluorescent images of P7 skin injected with different serotypes (n=3, GFP; green). FIG. 22C provides immunofluorescent images of P21 skin injected with different serotypes (n=3, GFP; green). FIG. 22D shows the tracking of AAV8-CAG-GFP transduced cells in PO injected mice (n=1, GFP; green).

FIGS. 23A-23E demonstrate that the combination of AAV-PHP.S and EF1a promoter showed tissue-specific transduction in APM and DP. FIG. 23A provides a schematic of a transduction pattern. FIG. 23B shows immunofluorescent staining for the transduced APM (RFP; green) from mice injected with AAV6-EF1a-DTA-RFP (left) and AAV-PHP.S-DTA-mCherry (right). FIG. 23C provides quantification of the RFP expressing APM in PO injected mice (n=2 for each condition, one-way ANOVA with Tukey's multiple comparisons test). FIG. 23D shows immunofluorescent staining for the transduced DP (RFP; green) from mice injected with AAV6-EF1a-DTA-RFP (left) and AAV-PHP.S-DTA-mCherry (right). FIG. 23E provides quantification of the RFP expressing DP in PO injected mice (n=2 for each condition, one-way ANOVA with Tukey's multiple comparisons test).

FIGS. 24A-24C demonstrate PO injection of AAV-PHP.S-DTA-mCherry ablated APM in Myh11-CreER mice. FIG. 24A provides an experimental scheme for PO injection and treatment with tamoxifen. FIG. 24B provides a comparison of APM in the control group (left) and experimental group (right). Both groups were injected with AAV-PHP.S-DTA-mCherry, but only the experimental (EXP) group was treated with tamoxifen. Immunofluorescent images are provided for the AAV transduction (RFP; green) and the APM ablation (SMA; red). FIG. 24C provides quantification of APM ablation in Myh11-CreER mice. In the control group, 9% showed partial ablation (n=3) and 91% remained normal. In the experimental group, 52% showed partial ablation (n=15), 7% showed full ablation (n=2), and 41% remained normal (n=12).

FIG. 25 shows intradermal injection of different AAV serotypes in adult mice. Total 4×E10 gc of AAV was injected in P21 mice. The skin samples were harvested at P27 and stained for different markers. Green: GFP; Red: ItgA8 for APM, CD31 for blood vessel, Pcad for epidermis, CD3 for immune cell, Perilipin for adipocytes, CD45 for immune cells.

FIG. 26 shows intradermal injection of different AAV serotypes in neonatal mice. Total 2×E10 gc of AAV was injected in PO mice. The skin samples were harvested at P7 and P21. Green: GFP; Red: Pcad for epidermis, CD26 for upper dermal fibroblast, SMA for smooth muscle actin, CD31 for blood vessel.

FIG. 27 demonstrates a test of different dosages in neonatal mice. Different dosages of AAV-CAG-GFP (2×E10, 2×E8 genomic copies) were administered in PO mice and harvested at P6. Among these conditions, 2×E10 genomic copies of AAV resulted in the most robust and widespread transduction in mice skin. GFP immunofluorescent staining (green) indicates AAV; CD3 for T cells; CD26 for upper dermal fibroblasts; CD31 for endothelial cells; CD45 for immune cells; CD140a for dermal fibroblasts; ITGA8 for arrector pili muscle; PCAD for epithelial and Perilipin for adipocytes.

FIGS. 28A-28B demonstrate an analysis of APM ablation in PO injected yhll-CreER mice of AAV-PHP.S-DTA-mCherry. FIG. 28A provides a comparison of APM ablation in the control group (top) and the experimental group (bottom). Immunofluorescent images for APM (SMA; green) and AAV transduction (RFP; red) are provided. FIG. 28B provides quantification of APM ablation in the control and experimental group.

FIGS. 29A-29C demonstrate AAVDJ- and AAV8-CAG-GFP and AAVPHP.S-CAG-tdTomato transduction of various cell types in the skin. FIG. 29A provides a vector map of AAV8 with a ubiquitous CAG promoter driving the expression of GFP. FIG. 29B shows immunofluorescence staining for Integrin A8 (alpha8), CD26, CD45, CD140a, and Perilipin A (Plpn), which demonstrates that AAVDJ serotype has a high transduction efficiency for adipocytes (last row), and AAV8 serotype highly transduces dermal fibroblasts (second/fourth row) and adipocytes (last row). FIG. 29C shows immunofluorescence staining for Integrin A8 (alpha8), CD26, CD31, CD45, CD140a, Perilipin A (Plpn), and tdTomato, which indicate that AAVPHP.S serotype is able to infect the arrector pili muscle (top row), dermal fibroblasts (second/fifth row), blood vessels (third row), and adipocytes (last row).

FIGS. 30A-30B demonstrate that the ubiquitous CAG and EF1a promoters (packaged in AAV8) have very similar transduction patterns with a few qualitative differences. FIG. 30A shows that there is no significant difference in transduction patterns between the CAG and EF1a promoters, but there does seem to be a general pattern of higher infectivity in the DP and APM by the EF1a promoter (n=1 mouse for each condition, 82 hair follicles/APM in AAV8-CAG condition and 73 hair follicles/APM in AAV8-EF1a condition, two tailed unpaired t-test). FIG. 30B shows immunofluorescence staining for Integrin A8 (alpha8), CD26, CD31, Pcad, and Perilipin A (Plpn) indicating similar transduction patterns between AAV8-CAG-GFP and AAV8-EF1a-GFP except for the dermal papillae, for which AAV8-Ef1a has a higher infectivity tendency (fourth row).

FIGS. 31A-31D demonstrate that AAV-mediated overexpression of Sonic Hedgehog (Shh) and Edn3 provide proof of concept and validation for discovering potential novel functions. FIG. 31A shows injection of AAV-Shh intradermally into the skin resulted in increased rate of anagen entry in the skin around the injection site 12 days after injection. FIG. 31B shows injection of AAV-Edn3 intradermally into the dorsal skin resulted in abnormal pigmentation in the ears 33 days after injection. FIG. 31C shows overexpression of Edn3 caused abnormal and ectopic pigmentation in and around the hair follicles 33 days after injection, FIG. 31D shows immunofluorescence staining for Perilipin A (Plpn) and Myc indicates that the AAV-Edn3 virus transduced mainly into adipocytes.

FIGS. 32A-32H demonstrate that Edn3 mediates the proliferation and migration of melanocyte into the epidermis and dermis. FIG. 32A provides a schematic of AAV-mediated overexpression of Edn3 to closely identify the stage at which Edn3 has the most effect. FIG. 32B shows Fontana-Masson staining to visualize abnormal pigmentation patterns in the skin of the AAV-Edn3 injected mouse. FIGS. 32C-32E provide immunofluorescence staining for EdU and TRP2, to demonstrate proliferation and migration into the dermis and epidermis of the skin. FIGS. 32F-32H show increased proliferating melanocyte numbers and migration in the AAV-Edn3 injected mouse compared to control (n=1 mouse for each condition, two-tailed unpaired t-test).

FIGS. 33A-33F demonstrate that AAV-mediated overexpression of Sonic Hedgehog (Shh) in wounded conditions does not lead to any apparent increase in wound healing. FIG. 33A provides immunofluorescence staining of GFP, which suggests that pipetting of AAV8-GFP into the wound area leads to infection of cells inside the wound area. FIG. 33B provides a schematic of AAV-mediated overexpression of Shh to determine differences in wound healing. FIGS. 33C-33D show that the wound does not experience a faster rate of re-epithelialization or healing with respect to the epidermis, but advanced hair stage in Shh-overexpressed condition confirms ectopic overexpression of Shh. FIG. 33E shows in situ hybridization of Shh further supports the successful overexpression of Shh in the dermis. FIG. 33F provides immunofluorescence staining for SMA and P-cadherin (Pcad), which shows abnormal hair growth and new hair follicle growth in the skin 33 days after wounding and introduction of AAV-Shh.

FIG. 34 provides immunofluorescence staining for GFP and SMA indicating that AAV8-CAG-GFP does not transduce into myofibroblasts or the APM in the wounded skin.

FIG. 35 provides immunofluorescence staining for CD3 and Pcad showing that there is no obvious difference in immune response 7 days after inoculation with AAV-Shh. Pcad staining supports advanced hair growth near the wound site with hair bulbs deeper in the dermis layer.

### DETAILED DESCRIPTION OF THE INVENTION

Disclosed herein are delivery systems (e.g., viral vector delivery systems) and methods that allow for the delivery of a gene to a specific cell type (e.g., a specific skin cell type). The viral vector delivery system described herein provides targeted delivery of one or more genes into a predetermined cell type for the purposes of treating one or more skin conditions.

The delivery systems (e.g., viral vector delivery systems) described herein may comprise an adeno-associated virus (AAV) and an enhancer or promoter for delivery of a gene or other deliverable. In some embodiments, the delivery system delivers a gene or other deliverable (e.g., a gene editing system or base editor) to a target cell, and in certain embodiments, the target cell is a skin cell. In some embodiments, the target cell is selected from the group consisting of dermal fibroblasts, dermal papilla, Schwann cells, adipocytes, dermal adipocytes, epidermal stem cells, hair follicle stem cells, melanocyte stem cells, nerve fibers, blood vessels, immune cells, arrector pili muscle (APM), panniculus carnosus, sympathetic nerves, sensory nerves, and pericytes. In other aspects, the delivery system comprises an AAV and an enhancer or promoter for delivery of a gene editing system (e.g., gRNA, shRNA, Cas9, or other Cas proteins) to edit a target site. In other aspects, the delivery system comprises an AAV and an enhancer or promoter for delivery of a base editor (e.g., a base editor that may convert, for example, an A to a G).

Adeno-associated virus (AAV) is a small (20 nm) replication-defective, nonenveloped virus. The AAV genome a single-stranded DNA (ssDNA) about 4.7 kilobase long. The genome comprises inverted terminal repeats (ITRs) at both ends of the DNA strand, and two open reading frames (ORFs): rep and cap. The AAV genome integrates most frequently into a particular site on chromosome 19 in humans. Random incorporations into the genome take place with a negligible frequency. The integrative capacity may be eliminated by removing at least part of the rep ORF from the vector resulting in vectors that remain episomal and provide sustained expression at least in non-dividing cells. To use AAV as a gene transfer vector, a nucleic acid comprising a nucleic acid sequence encoding a desired protein or RNA, e.g., encoding a polypeptide or RNA, operably linked to a promoter, is inserted between the inverted terminal repeats (ITR) of the AAV genome. Adeno-associated viruses (AAV) and their use as vectors, e.g., for gene therapy, are also discussed in Snyder, R O and Moullier, P., Adeno-Associated Virus Methods and Protocols, Methods in Molecular Biology, Vol. 807. Humana Press, 2011.

In some embodiments, the virus is AAV serotype 1, 2, 3, 3B, 4, 5, 6, 7, 8, 9, 10, 11, Anc80, or PHP.eB. (disclosed in US 2017/0166926, incorporated herein by reference). Any AAV serotype, or modified AAV serotype, may be used as appropriate and is not limited.

Another suitable AAV may be, e.g., Anc80 (i.e., Anc80L65) (WO2015054653) or rh10 (WO 2003/042397). Still other AAV sources may include, e.g., retro, PHP.B, PHP.S, hu37 (see, e.g. U.S. Pat. No. 7,906,111; US 2011/0236353), AAV1, AAV2, AAV3, AAV4, AAV5,

AAV6, AAV6.2, AAV7, AAV8, (US 7,790,449; US 7,282,199), AAV9 (US 7,906,111; US 2011/0236353), AAVrh10, AAV-DJ, AAV-DJ/8, AAV.CAP-B10, AAV.CAP-B22, AAVMYO, and others. See, e.g., WO 2003/042397; WO 2005/033321, WO 2006/110689; U.S. Pat. Nos. 7,790,449; 7,282,199; 7,588,772 for sequences of these and other suitable AAV, as well as for methods for generating AAV vectors. Still other AAVs may be selected, optionally taking into consideration cell preferences of the selected AAV capsid.

In some embodiments, a delivery system comprises a viral serotype selected from the group consisting of AAV2, AAV6, AAV8, AAV9, AAV-PHP.S, AAV-DJ, AAV-retro, and AAVrh10. In certain embodiments, a delivery system comprises a viral serotype selected from the group consisting of AAV8, AAV-PHP.S, AAV6, AAV-retro, and AAVrh10. In one embodiment, a delivery system comprises viral serotype AAV2. In one embodiment, a delivery system comprises viral serotype AAV8. In one embodiment, a delivery system comprises viral serotype AAV9. In one embodiment, a delivery system comprises viral serotype AAV-PHP.S. In one embodiment, a viral delivery system comprises viral serotype AAV6. In one embodiment, a viral delivery system comprises viral serotype AAV-retro. In one embodiment, a viral delivery system comprises viral serotype AAVrh10. In one embodiment, a delivery system comprises viral serotype AAV-DJ.

A recombinant AAV vector (AAV viral particle) may comprise, packaged within an AAV capsid, a nucleic acid molecule containing a 5′ AAV inverted terminal repeat (ITR), an expression cassette, and a 3′ AAV ITR. An expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3′ ITR. A shortened version of the 5′ ITR, termed AITR, has been described in which the D-sequence and terminal resolution site (trs) are deleted. The abbreviation “sc” refers to self-complementary. “Self-complementary AAV” refers to a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self- complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue or viral target. In one embodiment, the ITR sequences from AAV2, or the deleted version thereof (AITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2, the contents of which are incorporated herein by reference in their entirety. In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus ULS, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al, 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following

U.S. patents, the contents of which are incorporated herein by reference in their entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The delivery system may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EFlalpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, the promoter directs expression in a particular cell type (e.g., a targeted population of cells). In some embodiments, the promoter selectively directs expression in any population of cells described herein. In some embodiments, the promoter is a non-silencing promoter. In some embodiment, the promoter is selected from the group consisting of CAG, EF1, neuropeptide Y (NPY), and Human synapsin 1 gene promoter (hSyn). In one embodiment, a promoter is CAG. In one embodiment, a promoter is EF1 or EF1a. In one embodiment, a promoter is NPY. In one embodiment, a promoter is hSYN. In some embodiments, the promoter directs expression that is high, long-term, and uniform across the target cells.

In some embodiments, the gene is any gene to be delivered to a cell or tissue. In some embodiments, the gene is associated with a skin condition, disease, or disorder. Genes may be identified utilizing the OMIM database available at omim.org. In some embodiments, the gene is selected from the group consisting of FGF18, DTA, DREADDS, Gas6, SHH, Noggin, BMP2, BMP4, FGF7, and FGF10. Additional examples of genes that may be delivered to skin cells using the delivery system are described in “The Genetics of Human Skin Disease,” Cold Spring Harb Perspect Med 4(10) 2014, the entirety of which is incorporated herein by reference.

In some embodiments, a delivery system comprises an AAV2 serotype and a CAG promoter. In some embodiments, a delivery system comprises an AAV9 serotype and a CAG promoter. In some embodiments, a delivery system comprises an AAV8 serotype and a CAG promoter, e.g., for delivery of a gene to a subject. In some embodiments, a delivery system comprises an AAV8 serotype and an EF1 promoter. In some embodiments, a delivery system comprises an AAVrh10 serotype and a CAG promoter. In some embodiments, a delivery system comprises an AAV6 serotype and a CAG promoter. In some embodiments, a delivery system comprises an AAV6 serotype and an EF1 promoter. In some embodiments, a delivery system comprises an AAV-PHP.S serotype and a CAG promoter. In some embodiments, a delivery system comprises an AAV-PHP.S serotype and a EF1 promoter. In some embodiments, a delivery system comprises an AAV-PHP.S serotype and an NPY promoter. In some embodiments, a delivery system comprises an AAV-PHP.S serotype and a hSYN promoter. In some embodiments, a delivery system comprises an AAV-retro serotype and a CAG promoter. In some embodiments, a delivery system comprises an AAV-retro serotype and a hSYN promoter. In some embodiments, a delivery system comprises an AAV-DJ serotype and a CAG promoter.

The delivery system may result in overexpression of a native gene by at least 10%, 15%, 20%, 25%, 30%, 35%, 40%, 45%, 50%, 55%, 60%, 65%, 70%, or 75% of wild-type levels in a target cell or tissue (e.g., in at least 70% of fat free, blood free body mass). In some embodiments, the delivery system may result in overexpression of a native gene by at least 100%, 200%, 300%, 400%, 500%, 600%, 700%, 800%, 900%, 1000%, 1500%, 2000%, 2500%, 5000%, 7500%, 10000%, 50000%, 100000% of wild-type levels in a target cell or tissue. In some embodiments, the delivery system delivers a native gene resulting in overexpression of the native gene by about 10%-90%, 20%-80%, 30%-70%, or 40%-60% of wild-type levels in a tissue. In some embodiments, the delivery system results in overexpression of a native gene by at least 30%, or by about 25-50%, of wild-type levels. The delivery system may result in detectable expression (e.g., greater than trace expression) of a non-native gene in a target cell or tissue (e.g., in at least 70% of fat free, blood free body mass). In some embodiments, expression of the delivered gene is stable and long-term (e.g., expression is maintained for at least 1 month, 2 months, 3 months, 4 months, 5 months, 6 months, 7 months, 8 months, 9 months, 10 months, 11 months, 12 months, 15 months, 18 months, 21 months, 24 months, 3 years, 4 years, 5 years, 10 years, 15 years, 20 years, 30 years, 40 years, 50 years, 60 years, 70 years, 80 years, 90 years).

In some embodiments, the delivery system delivers a gene of interest to a cell or tissue of interest (e.g., dermal fibroblasts, dermal papilla, Schwann cells, adipocytes, dermal adipocytes, epidermal stem cells, hair follicle stem cells, melanocyte stem cells, nerve fibers, blood vessels, immune cells, arrector pili muscle (APM), panniculus carnosus, and sympathetic nerves). In some embodiments, the delivery system delivers a gene of interest to multiple cells or tissues of interest in a subject. For example, the delivery system may deliver a gene of interest to at least 20%, 30%, 40%, 50%, 60%, 70%, 80%, or 90% of cells or tissues in a subject. In some embodiments, the delivery system delivers a gene to about 10%-90%, 20%-80%, 30%-70%, or 40%-60% of cells or tissues in the subject. The delivery system may provide uniform or limited variable delivery of a gene across multiple cells or tissues within a subject.

In some embodiments, a delivery system comprises an AAV2 serotype and a CAG promoter for delivery of a gene to one or more cells comprising adipocytes.

In some embodiments, a delivery system comprises an AAV9 serotype and a CAG promoter for delivery of a gene to one or more cells selected from the group consisting of adipocytes, fibroblasts, and arrector pili muscle.

In some embodiments, a delivery system comprises an AAV-DJ serotype and a CAG promoter for delivery of a gene to one or more cells comprising adipocytes.

In some embodiments, a delivery system comprises an AAV8 serotype and a CAG promoter for delivery of a gene (e.g., intradermally) to one or more cells selected from the group consisting of fibroblasts, dermal papilla, adipocytes, arrector pili muscle, and immune cells. In one embodiment, a delivery system comprises an AAV8 serotype and a CAG promoter for delivery of FGF18, e.g., to a hair follicle stem cell. In one embodiment, a delivery system comprises an AAV8 serotype and a CAG promoter for delivery of an Edn3 gene. In one embodiment, a delivery system comprises an AAV8 serotype and a CAG promoter for delivery of a SHH gene. In some aspects, a delivery system comprising an AAV8 serotype and a CAG promoter deliver a gene to a cell (e.g., intradermally) that is not a sensory nerve or a sympathetic nerve. In some embodiments, a delivery system comprises an AAV8 serotype and an EF1 promoter for delivery of a gene (e.g., intradermally) to one or more cells selected from the group consisting of fibroblasts, adipocytes, arrector pili muscle, and immune cells. In some aspects, a delivery system comprising an AAV8 serotype and a EF1 promoter deliver a gene to a cell (e.g., intradermally) that is not a sensory nerve or a sympathetic nerve. In one embodiment, a delivery system comprises an AAV8 serotype and an EF1 promoter for delivery of DTA to one or more cells.

In some embodiments, a delivery system comprises an AAVrh10 serotype and a CAG promoter for delivery of a gene (e.g., intradermally) to one or more cells selected from the group consisting of fibroblasts, adipocytes, and arrector pili muscle. In some aspects, a delivery system comprising an AAVrh10 serotype and a CAG promoter deliver a gene to a cell (e.g., intradermally) that is not a sensory nerve or a sympathetic nerve.

In some embodiments, a delivery system comprises an AAV6 serotype and a CAG promoter for delivery of a gene (e.g., intradermally) to one or more cells selected from the group consisting of fibroblasts, adipocytes, and arrector pili muscle. In some aspects, a delivery system comprising an AAV6 serotype and a CAG promoter deliver a gene to a cell (e.g., intradermally) that is not a sensory nerve or a sympathetic nerve. In some embodiments, a delivery system comprises an AAV6 serotype and a EF1 promoter for delivery of a gene to one or more cells selected from the group consisting of adipocytes and arrector pili muscle. In embodiment, a delivery system comprises an AAV6 serotype and a EF1 promoter for delivery of DTA to one or more cells.

In some embodiments, a delivery system comprises an AAV-PHP.S serotype and a CAG promoter for delivery of a gene (e.g., intradermally) to one or more cells selected from the group consisting of fibroblasts, adipocytes, arrector pili muscle, and panniculus carnosus. In one embodiment, a delivery system comprises an AAV-PHP.S serotype and a CAG promoter for delivery of DTA, e.g., to an arrector pili muscle or a fibroblast. In some aspects, a delivery system comprising an AAV-PHP.S serotype and a CAG promoter deliver a gene to a cell (e.g., intradermally) that is not a sensory nerve or a sympathetic nerve. In some embodiments, a delivery system comprises an AAV-PHP.S serotype and a CAG promoter for delivery of a gene (e.g., intravenously) to one or more cells selected from the group consisting of fibroblasts, adipocytes, sensory nerves, and sympathetic nerves. In some aspects, a delivery system comprising an AAV-PHP.S serotype and a CAG promoter deliver a gene to a cell (e.g., intravenously) that is not an arrector pili muscle or panniculus carnosus. In some embodiments, a delivery system comprises an AAV-PHP.S serotype and an EF1 promoter for delivery of a gene (e.g., intradermally) to one or more cells selected from the group consisting of fibroblasts, dermal papilla, adipocytes, and arrector pili muscle. In one embodiment, a delivery system comprises an AAV-PHP.S serotype and an EF1 promoter for delivery of DTA to one or more genes. In some aspects, a delivery system comprising an AAV-PHP.S serotype and an EF1 promoter deliver a gene to a cell (e.g., intradermally) that is not a sensory nerve or a sympathetic nerve. In some embodiments, a delivery system comprises an AAV-PHP.S serotype and an NPY promoter for delivery of a gene (e.g., intradermally) to one or more cells selected from the group consisting of sensory nerves and sympathetic nerves. In some aspects, a delivery system comprising an AAV-PHP.S serotype and an NPY promoter deliver a gene to a cell (e.g., intradermally) that is not dermal papilla or arrector pili muscle. In some embodiments, a delivery system comprises an AAV-PHP.S serotype and a hSYN promoter for delivery of a gene (e.g., intradermally) to one or more cells selected from the group consisting of sensory nerves and sympathetic nerves. In some aspects, a delivery system comprising an AAV-PHP.S serotype and an hSYN promoter deliver a gene to a cell (e.g., intradermally) that is not dermal papilla or arrector pili muscle.

In some embodiments, a delivery system comprises an AAV-retro serotype and a CAG promoter for delivery of a gene (e.g., intradermally) to one or more cells selected from the group consisting of adipocytes and sympathetic nerves. In some aspects, a delivery system comprising an AAV-retro serotype and a CAG promoter deliver a gene to a cell (e.g., intradermally) that is not fibroblasts, dermal papilla, arrector pili muscle, or sensory nerves. In some embodiments, a delivery system comprises an AAV-retro serotype and a hSYN promoter for delivery of a gene (e.g., intradermally) to sympathetic nerves. In one embodiment, a delivery system comprises an AAV-retro serotype and a hSYN promoter for delivery of DTA to ablate sympathetic neurons innervation of the skin. In one embodiment, a delivery system comprises an AAV-retro serotype and a hSYN promoter for delivery of DREADDS to modulate the activity of sympathetic neurons. In some aspects, a delivery system comprising an AAV-retro serotype and an hSYN promoter deliver a gene to a cell (e.g., intradermally) that is not fibroblasts, dermal papilla, adipocytes, arrector pili muscle, or sensory nerves.

Some embodiments of the present invention relate to methods of treatment or prevention for a disease or condition, such as a skin condition, disease, or disorder, by the delivery of a pharmaceutical composition comprising an effective amount of the delivery system described herein. An effective amount of the pharmaceutical composition is an amount sufficient to prevent, slow, inhibit, or ameliorate a disease or disorder in a subject to whom the composition is administered. In some embodiments, the delivery of a pharmaceutical composition comprising an effective amount of the delivery system described herein extends the life expectancy or lifespan of a subject.

In some embodiments, the delivery system is administered to a subject. The delivery system may deliver a gene to a subject, e.g., to one or more cells or tissues of a subject. In some embodiments, the subject is expected to suffer from a disease or disorder based on family history or genetic analysis but is not currently suffering from the disease or disorder. In some embodiments, the subject is suffering from a disease or disorder.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient”, “individual” and “subject” are used interchangeably herein. Preferably, the subject is a mammal. The mammal can be a human, non-human primate, mouse, rat, dog, cat, horse, or cow, but are not limited to these examples. In addition, the methods described herein can be used to treat domesticated animals and/or pets. A subject can be male or female. A subject can be one who has been previously diagnosed with or identified as suffering from or having a condition in need of treatment or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. Rather, a subject can include one who exhibits one or more risk factors for a condition or one or more complications related to a condition. A “subject in need” of treatment for a particular condition can be a subject having that condition, diagnosed as having that condition, or at increased risk of developing that condition relative to a given reference population.

As used herein, “treat,” “treatment,” “treating,” or “amelioration” when used in reference to a disease, disorder or medical condition, refer to therapeutic treatments for a condition, wherein the object is to prevent, reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state as compared to that expected in the absence of treatment.

The efficacy of a given treatment for a disorder or disease can be determined by the skilled clinician. However, a treatment is considered “effective treatment,” as the term is used herein, if any one or all of the signs or symptoms of a disorder are altered in a beneficial manner, other clinically accepted symptoms are improved or ameliorated, e.g., by at least 10% following treatment with an agent or composition as described herein. Efficacy can also be measured by a failure of an individual to worsen as assessed by hospitalization or need for medical interventions (i.e., progression of the disease is halted). Methods of measuring these indicators are known to those of skill in the art and/or described herein.

In accordance with methods of the invention, treatment comprises contacting one or more cells or tissues with a composition according to the invention. The routes of administration will vary and include, e.g., intradermal, transdermal, parenteral, intravenous, intramuscular, intranasal, subcutaneous, regional, percutaneous, intratracheal, intraperitoneal, intraarterial, intravesical, intraocular, intratumoral, inhalation, perfusion, lavage, and oral administration and formulation. Treatment regimens may vary as well, and often depend on disease type, disease location, disease progression, and health and age of the patient.

The treatments may include various “unit doses” defined as containing a predetermined-quantity of the therapeutic composition. The quantity to be administered, and the particular route and formulation, are within the skill of those in the clinical arts. A unit dose need not be administered as a single injection but may comprise continuous infusion over a specified period of time. The dosage ranges for the agent depends upon the potency and the ability to produce the desired effect. The dosage should not be so large as to cause unacceptable adverse side effects.

Injection of the delivery system may be delivered by syringe or any other method used for injection of a solution, as long as the delivery system can pass through the particular gauge of needle required for injection and the dosage can be administered with the required level of precision.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this aspect, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The phrase “pharmaceutically-acceptable” or “pharmacologically-acceptable” refers to molecular entities and compositions that do not produce an allergic or similar untoward reaction when administered to a subject. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Except insofar as any conventional media or agent is incompatible with the viral agent, its use in the therapeutic compositions is contemplated. Supplementary active ingredients can also be incorporated into the compositions.

In some embodiments, the methods further comprise administering the pharmaceutical composition described herein along with one or more additional agents, biologics, drugs, or treatments beneficial to a subject suffering from a disorder or disease.

In some embodiments, the delivery system or pharmaceutical compositions comprising the delivery system are administered to a subject to treat a disease or condition.

In some embodiments, the disease or condition is a skin disease or condition. In certain aspects, the skin disease or condition is an autoinflammatory/autoimmune disease or condition. Non-limiting examples of the disease or condition include hair follicle regeneration, wound healing, melanocyte maintenance, epidermolysis bullosa, epidermolysis bullosa simplex, epidermolytic hyperkeratosis, dystrophic epidermolysis bullosa, epidermolytic palmoplantar keratoderma, Hailey-Hailey disease, Darier's disease, autosomal recessive hypotrichosis, pachyonychia congenita, melanoma, ichthyosis, seroderma pigmentosum, keratoderma, psoriasis, systemic lupus erythematosus, androgenetic alopecia, atopic dermatitis, systemic sclerosis, vitiligo, alopecia areata, pemphigus vulgaris, foliaceus, Sjorgren's syndrome, and netherton syndrome. Additional diseases and conditions are described in “The Genetics of Human Skin Disease,” Cold Spring Harb Perspect Med 4(10) 2014, the entirety of which is incorporated herein by reference.

In some embodiments, a delivery system or a pharmaceutical composition comprising the delivery system is administered (e.g., intravenously or intradermally) to a subject. The delivery system may deliver a gene, e.g., FGF18 or DTA, to the subject to treat a disease or condition.

Also disclosed herein are methods of encouraging hair growth in a subject. The methods may include elevating sympathetic nerve activity. In some embodiments, sympathetic nerve activity is elevated by exposing the subject to a cold temperature for a minimum period of time. In some embodiments, the exposure of the subject to the cold temperature activates the hair follicle stem cells and/or results in enhanced c-Fos expression.

In some embodiments, the subject is exposed to a cold temperature of about 5° C., 4° C., 4° C., 3° C., 2° C., 1° C., 0° C., −1° C. , -2° C. , -3° C. , -4° C., or -5° C. In some embodiments, the subject is exposed to a cold temperature of less than 5° C. In some embodiments, the subject is exposed to a cold temperature for at least 2 hours, 3 hours, 4 hours, 5 hours, 6 hours, 7 hours, 8 hours, 9 hours, or 10 hours. In some embodiments, the cold temperature is applied directly and/or specifically to the location of desired hair growth, e.g., the scalp.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited.

It should also be understood that unless otherwise indicated or evident from the context, any product or composition described herein may be considered “isolated”.

### EXEMPLIFICATION

### Example 1

### Cell Types Promoting Goosebumps Form a Niche to Regulate Hair Follicle Stem Cells

### Summary

Piloerection (goosebumps) requires concerted actions of the hair follicle, the arrector pili muscle (APM), and the sympathetic nerve, providing a model to study interactions across epithelium, mesenchyme, and nerves. Here, it was shown that APMs and sympathetic nerves form a dual component niche to modulate hair follicle stem cell (HFSC) activity. Sympathetic nerves form synapse-like structures with HFSCs and regulate HFSCs through norepinephrine, whereas APMs maintain sympathetic innervation to HFSCs. Without norepinephrine signaling, HFSCs enter deep quiescence by down-regulating cell cycle and metabolism while up-regulating quiescence regulators Foxp1 and Fgf18. During development, HFSC progeny secretes Sonic Hedgehog (SHH) to direct the formation of this APM-sympathetic nerve niche, which in turn controls hair follicle regeneration in adults. The results reveal a reciprocal interdependence between a regenerative tissue and its niche at different stages and demonstrate sympathetic nerves can modulate stem cells through synapses-like connections and neurotransmitters to couple tissue production with demands.

### Introduction

Cell types from multiple lineages assemble into specific arrangements in organs. The functions of these cell types must be integrated to enable optimal outcomes in tissue homeostasis, maintenance, and function in an ever-changing environment, although the mechanisms of such integration are not well understood. Epithelium, mesenchyme, and nerves are principle components of all organs. The sympathetic nervous system is a branch of the autonomic nervous system critical for maintaining body physiology under steady state and mediating “fight-or-flight” responses following external insults. The cell bodies of sympathetic neurons reside in the sympathetic ganglia close to the spinal cord, while the axons extend out and innervate essentially all organs (Borden et al., 2013; Karemaker, 2017; Suo et al., 2015). Under steady state, the sympathetic neurons are active within a basal range to maintain diverse processes including heart rate, respiration and blood pressure. External stimuli, such as cold or danger, elevate the sympathetic nerve activity to different degrees according to the strength of the insult, allowing rapid changes in body physiology that enable animals to respond.

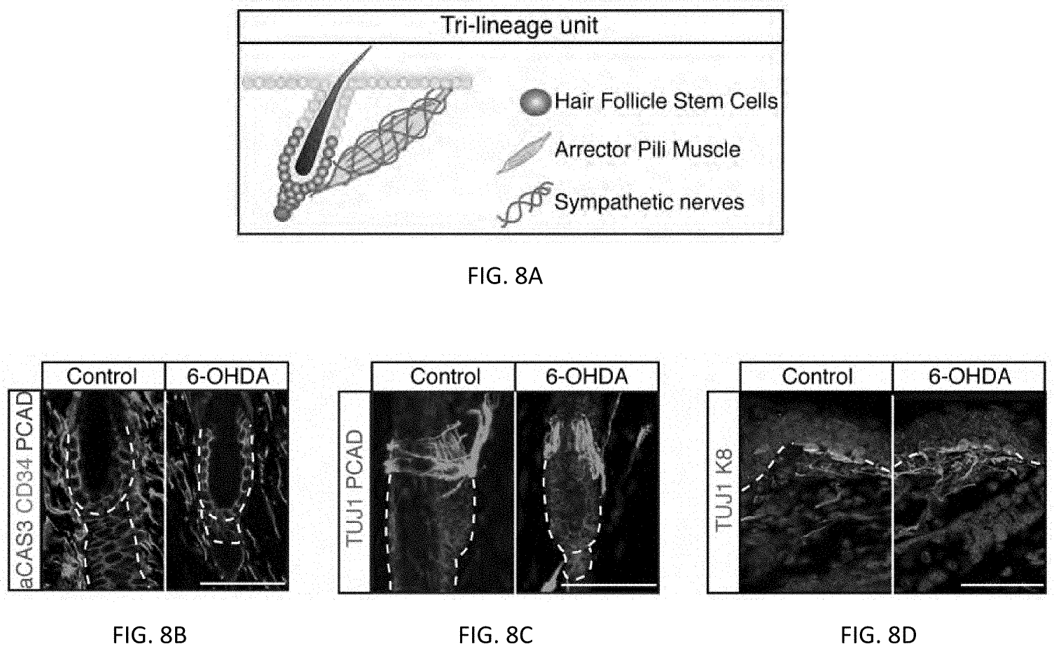

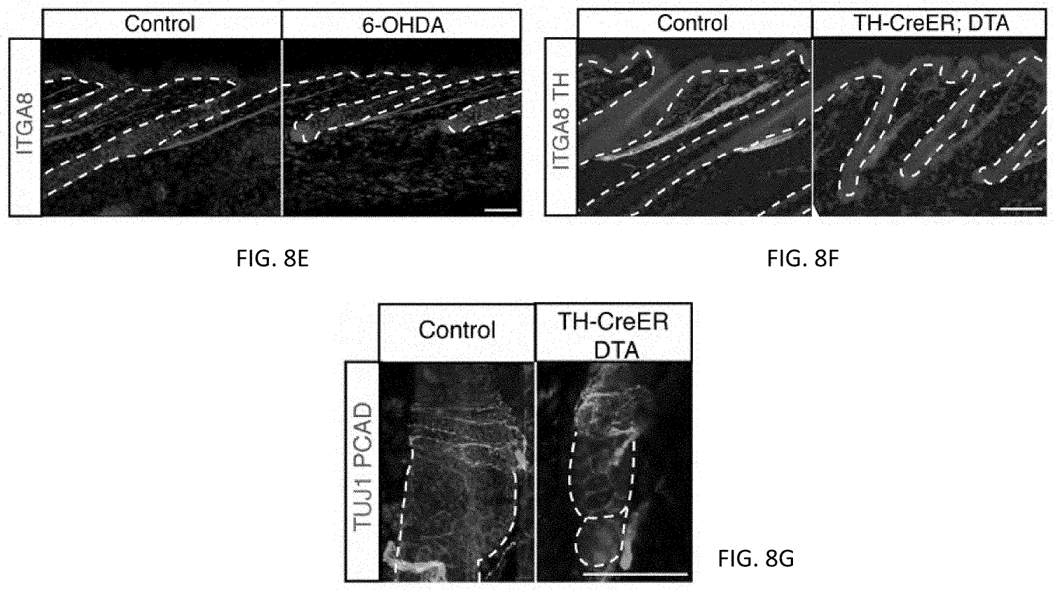

In the skin, the sympathetic innervation together with the arrector pili muscle (APM, mesenchymal origin) and the hair follicle (epithelial origin) form a tri-lineage unit (FIG. 8A). The sympathetic nerve innervates APMs, which are bundles of smooth muscle cells (Furlan et al., 2016). APMs are attached to the bulge region of the hair follicle where hair follicle stem cells (HFSCs) reside (Fujiwara et al., 2011). Environmental stimuli, such as cold temperatures, have a pronounced effect on the tri-lineage unit: elevated impulses from sympathetic nerves trigger the contraction of APM bundles, pulling the hair erect, a phenomenon known as piloerection or goosebumps. The erected hair traps air to create a layer of insulation for thermoregulation. Besides piloerection, it is unclear whether there are other functions of this tri-lineage unit. Yet, this tri-lineage configuration is highly conserved across mammals including in humans, where piloerection has lost its role in thermoregulation, raising the possibility of additional functions.

Insight into such additional functions comes from the observation that changes in hair growth and changes in the sympathetic nervous system are often linked. The hair follicle undergoes rounds of rest (telogen) and growth (anagen), known as the hair cycle (Muller-Rover et al., 2001). HFSCs in the bulge and hair germ remain quiescent throughout most hair cycles, but become proliferative transiently at anagen onset to produce their transit-amplifying progeny—the matrix, which then undergoes massive proliferation and differentiation to fuel the growth of new hair (Greco et al., 2009; Hsu et al., 2014b; Lay et al., 2016; Rompolas et al., 2013; Wang et al., 2016; Zhang and Hsu, 2017; Zhang et al., 2009). It is known that loss of sympathetic innervation is associated with defects in hair growth in diverse organisms (Asada-Kubota, 1995; Botchkarev et al., 1999; Crowe et al., 1993; Kobayasi et al., 1958; Kong et al., 2015; Peters et al., 1999). In addition, adrenergic agonists promote anagen hair follicle growth in cultured skin explants, and external light stimulates hair growth via the sympathetic nervous system (Botchkarev et al., 1999; Fan et al., 2018). These findings raise several key questions. First, given the broad impact of the sympathetic nervous system on whole body physiology and the diverse cell types that influence hair growth at the level of stem cell or niche, what are the direct cellular targets of the sympathetic neurons in hair growth control? Second, what are the cellular and molecular mechanisms by which sympathetic nerves regulate hair growth? Third, given that APMs are part of this tri-lineage unit, is there a role for APMs in regulating hair growth?

Here, these questions are addressed by combining cell-type specific gene deletion, cell ablation, transcriptome profiling, high-resolution imaging, and three-dimensional electron microscopy (3D-EM). It was shown that APMs are crucial for the formation and maintenance of sympathetic innervation to HFSCs, which allows sympathetic innervation to activate HFSCs directly through synapse-like connections that deliver the neurotransmitter norepinephrine. Under steady state, this nerve-APM-HFSC connection primes HFSCs for activation by lowering the expression of quiescence regulators Foxpl and Fgf18. Under cold conditions, the sympathetic nervous system is elevated, triggering not only goosebumps but also accelerating HFSC activation to produce new hair coat, coupling tissue growth with environmental changes. During development, the developing hair follicle initiates the formation of this tri-lineage unit through Sonic Hedgehog (SHH). Together, the findings illustrate an example of how cell types from the epithelium, mesenchyme, and nerve are integrated to allow tissue maintenance and function during development, homeostasis, and in response to environmental stimuli.

### Results

### Sympathetic Nerve Activity Regulates HFSC Activity

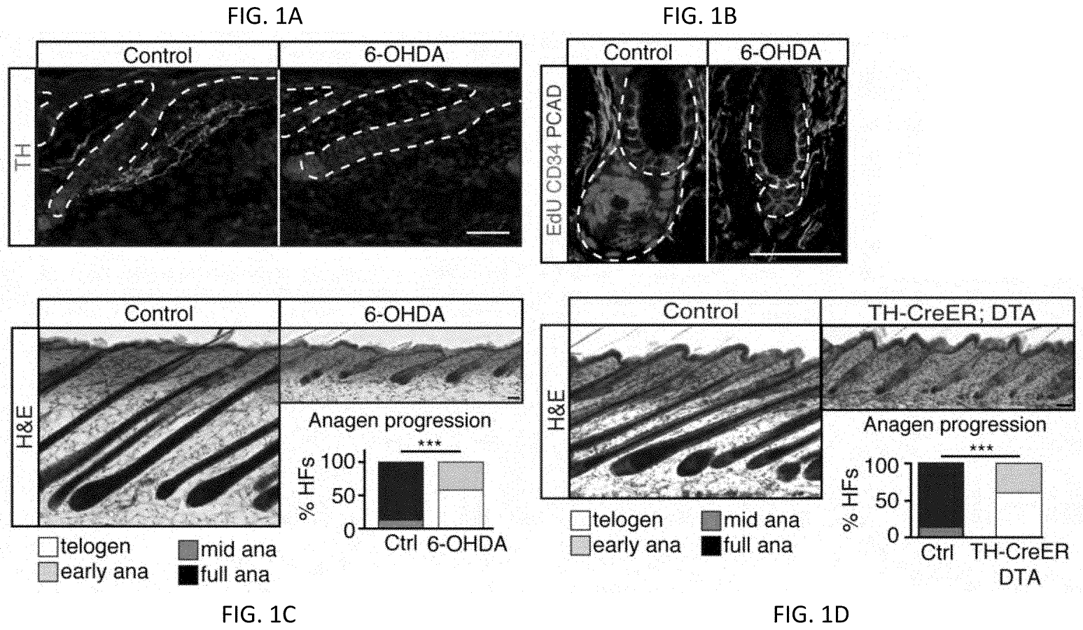

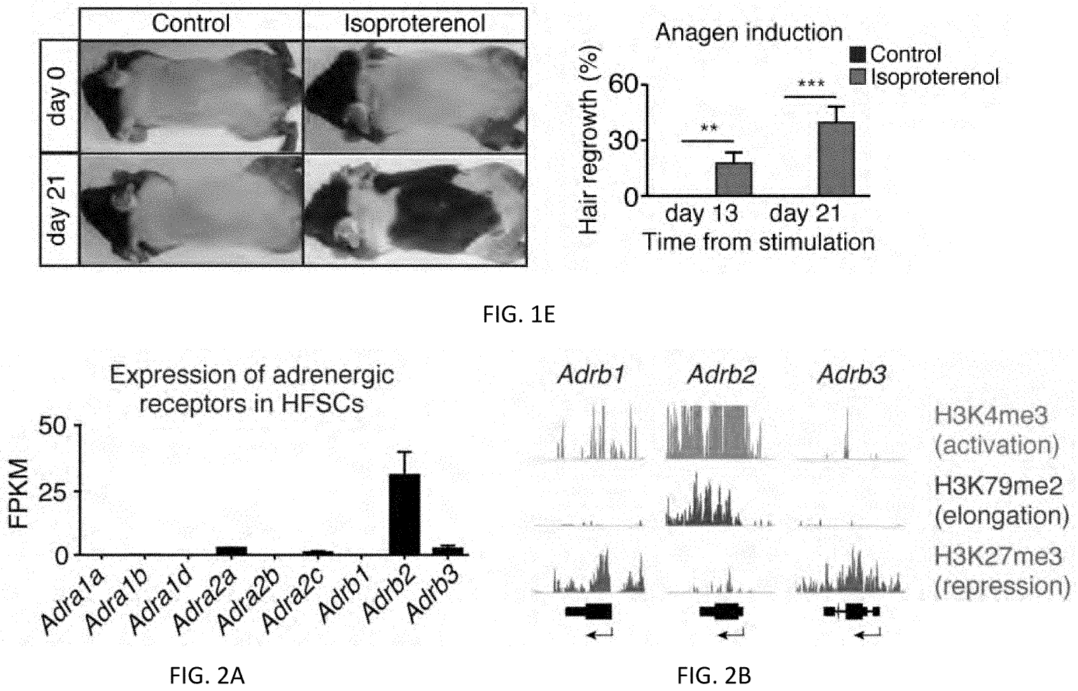

The sympathetic nervous system is constantly active at a basal level to maintain body physiology. To explore if basal sympathetic nerve activity affects the other two cell types in this nerve-APM-HFSC tri-lineage unit, the sympathetic nerve in telogen skin was ablated through intradermal injection of 6-hydroxydopamine (6-OHDA, a selective neurotoxin for sympathetic nerves) (Kostrzewa and Jacobowitz, 1974), when HFSCs are quiescent. 6-OHDA ablated the sympathetic nerve efficiently, but spared the sensory nerves and other cell types in the skin (FIG. 1A and FIGS. 8B-8E). Sympathectomy did not cause noticeable changes in APMs, but led to a substantial delay in HFSC activation and anagen entry (FIG. 1B and FIG. 8E). By P30, hair follicles reached full anagen throughout the control back skin, whereas hair follicles in the sympathectomized skin remained mostly in telogen (FIG. 1C). Similar results were obtained with a genetic model of sympathectomy by topical application of 4-OH-tamoxifen on TH-CreER; Rosa-lsl-attenuated Diphtheria toxin fragment A (DTA) mice (FIG. 1D, FIGS. 8F-8G). These results suggest that basal sympathetic nerve activity is required for HFSC activation and anagen entry but dispensable for APM maintenance.

Next, the impact of elevated sympathetic tone on HFSC activation was explored. Sympathetic nerve terminals secrete the neurotransmitter norepinephrine, which binds to adrenergic receptors on target cells. To elevate the sympathetic tone, isoproterenol, a pan-adrenergic receptor agonist, was topically applied at the extended 2^{nd} telogen. Mice with topical application of isoproterenol entered anagen earlier (FIG. 1E). Collectively, these data suggest that HFSC activity is tightly linked with sympathetic nerve activity. Loss of sympathetic nerve innervations makes HFSCs more dormant, whereas elevated sympathetic tone promotes HFSC activation.

### The Sympathetic Nerve Regulates HFSCs Directly Through Norepinephrine-Adrb2 Signaling

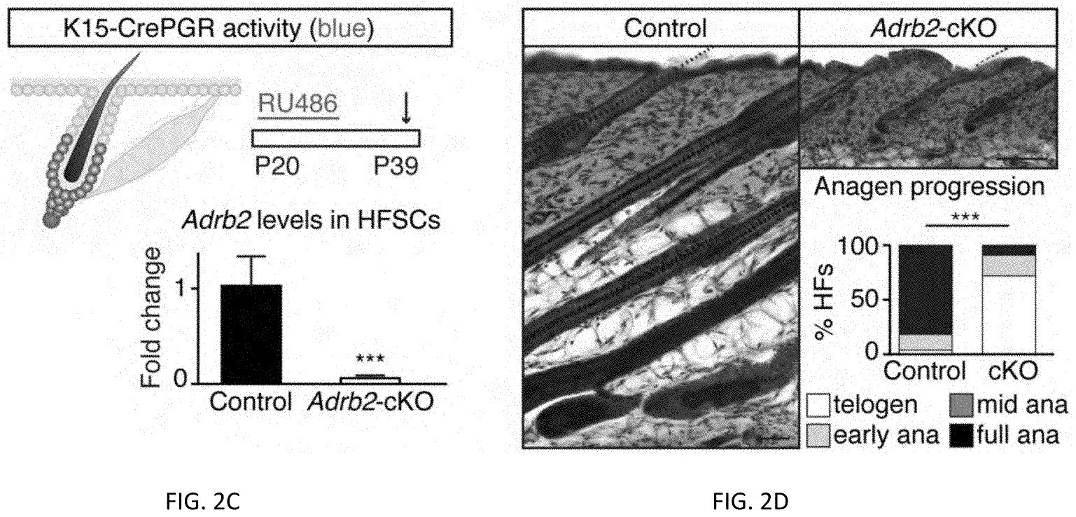

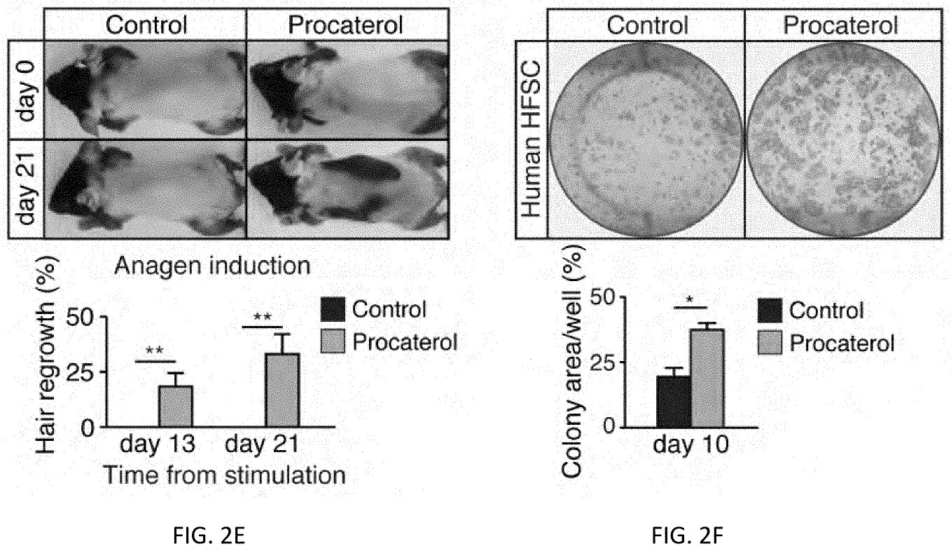

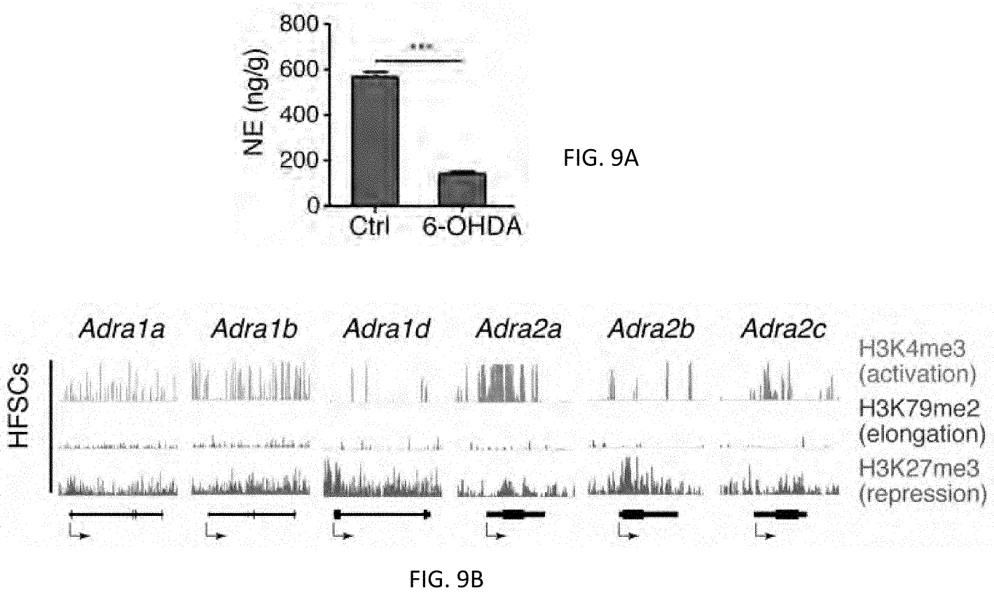

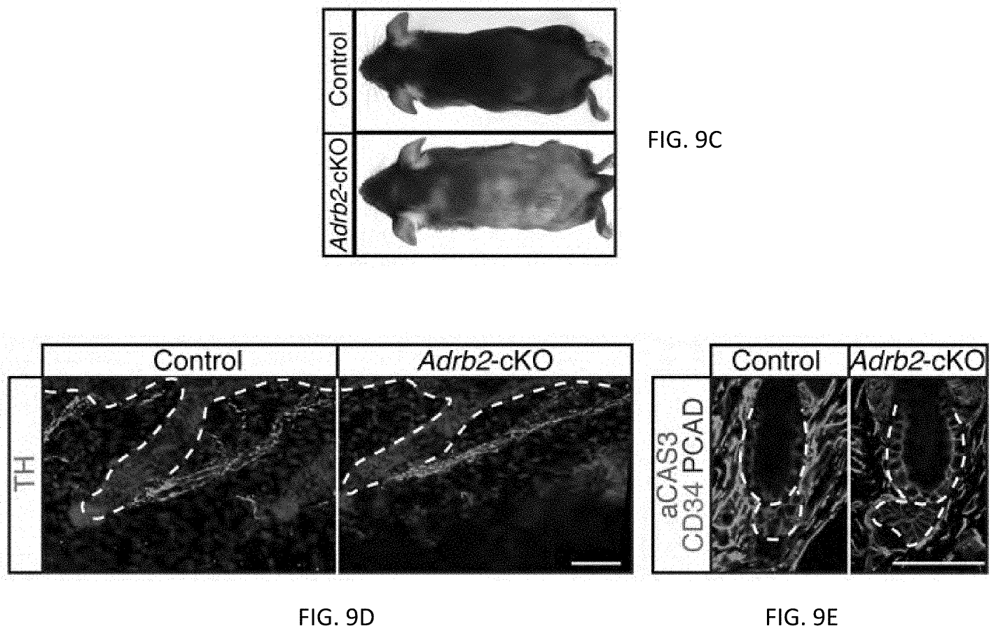

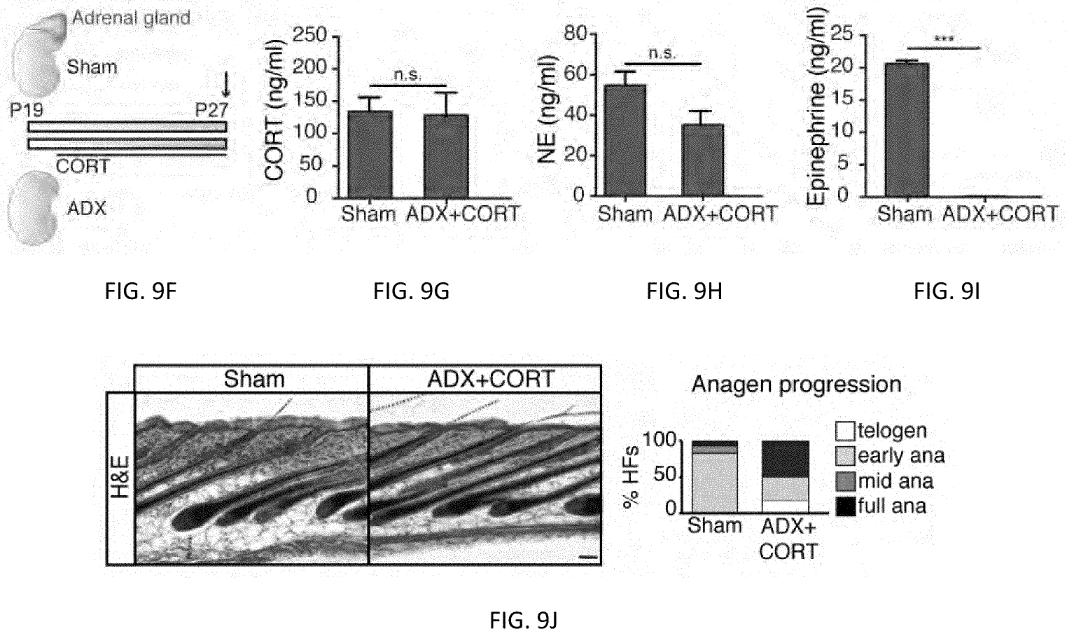

Whether the sympathetic nerve regulates HFSCs directly was then tested. Sympathectomy led to diminished norepinephrine levels in the skin, suggesting that the sympathetic nerve is indeed a key source of norepinephrine in the skin (FIG. 9A). By surveying RNA-sequencing (RNA-seq) and ChIP-seq datasets (Ge et al., 2017; Lay et al., 2016; Lien et al., 2011), it was found that Adrb2 is the predominant adrenergic receptor expressed in HFSCs (FIGS. 2A-2B and FIG. 9B). To test if norepinephrine acts directly on HFSCs, K15-CrePGR; Adrb2 fl/fl mice were generated, in which Adrb2 is depleted only from HFSCs (FIG. 2C). Adrb2-cKO mice displayed significantly extended telogen length similar to the sympathectomized mice, suggesting that Adrb2-cKO HFSCs are refractory to activation (FIG. 2D and FIG. 9C). Other than the delayed anagen entry, Adrb2-cKO mice showed no changes in sympathetic innervation and no signs of abnormal cell death (FIGS. 9D-9E). Moreover, topical application of an ADRB2-specific agonist (procaterol) at the extended 2^{nd} telogen accelerated anagen entry (FIG. 2E). These data suggest that loss of ADRB2 inhibits HFSC activation, whereas elevation of ADRB2 activity promotes HFSC activation. Moreover, addition of procaterol onto cultured human HFSCs promoted their growth, suggesting the pathway has a conserved function in regulating human HFSCs (FIG. 2F).

ADRB2 binds to both norepinephrine and epinephrine. In addition to the sympathetic nerve, adrenal glands secrete epinephrine and norepinephrine (collectively known as catecholamines) into the bloodstream. To test if adrenal gland-derived catecholamines regulate HFSCs, adrenal glands were removed and corticosterone was supplied back, another adrenal gland-derived hormone (FIGS. 9F-9I). Unlike sympathectomized mice, adrenalectomized mice supplemented with corticosterone had no delays in anagen entry (FIG. 9J), suggesting that adrenal gland-derived catecholamines are dispensable for HFSC activation. These data establish that the sympathetic nerve secretes norepinephrine, which binds to ADRB2 on HFSCs to modulate stem cell activity directly.

### Transcriptomic Analyses of Adrb2-Deficient HFSCs

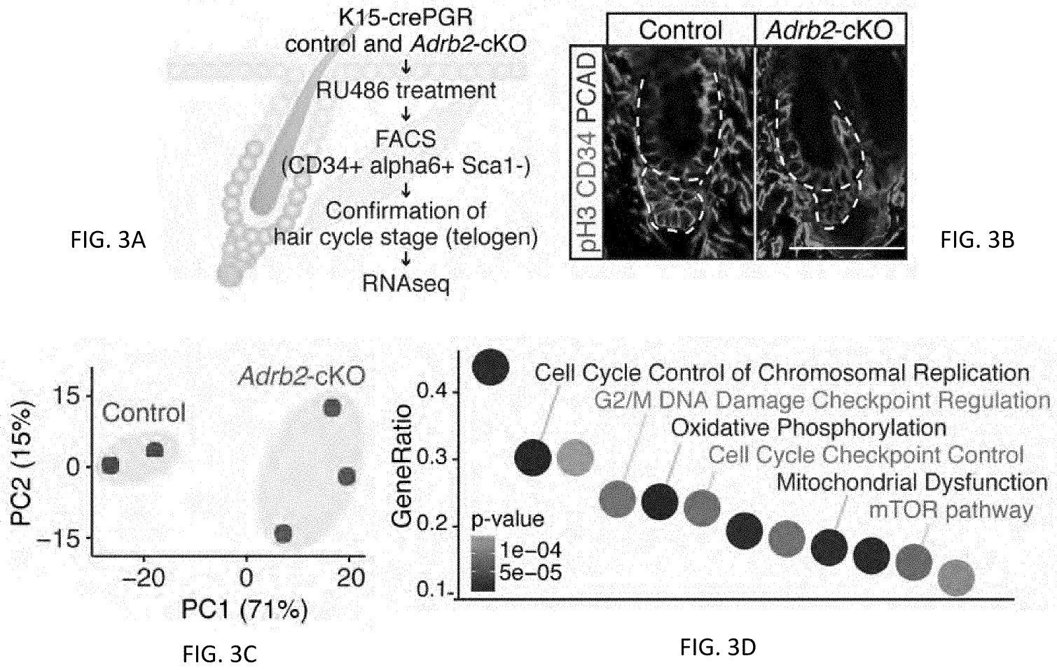

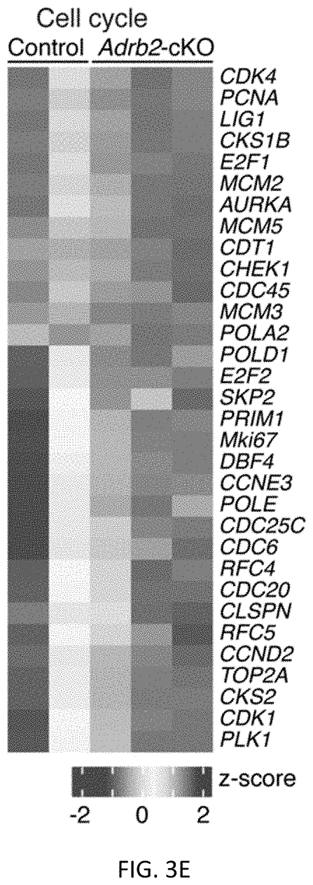

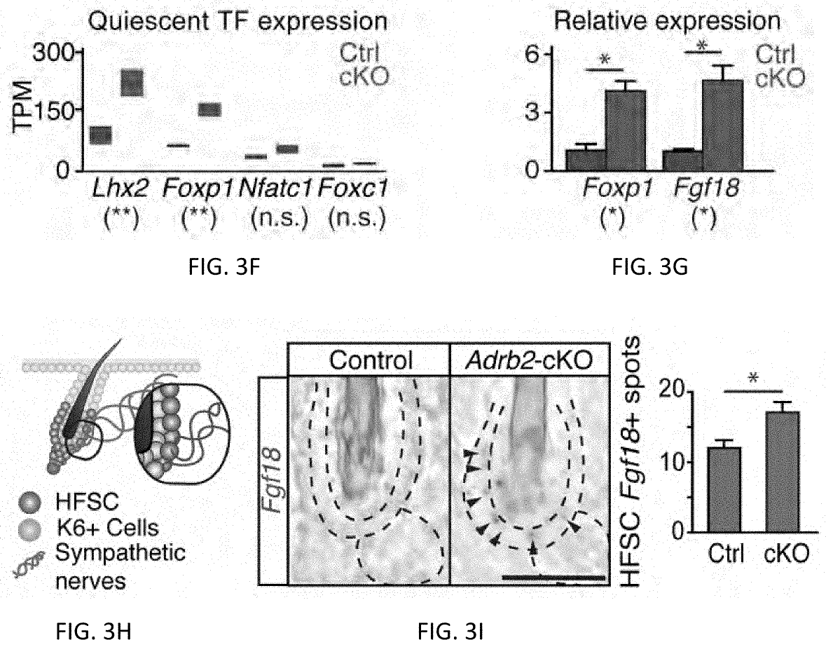

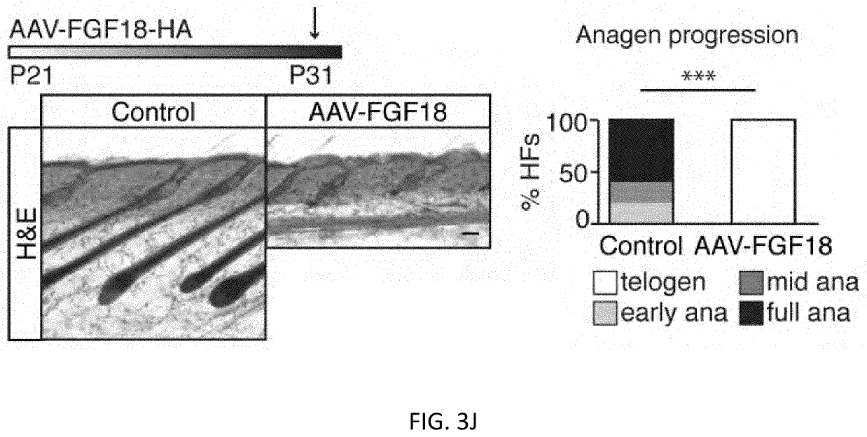

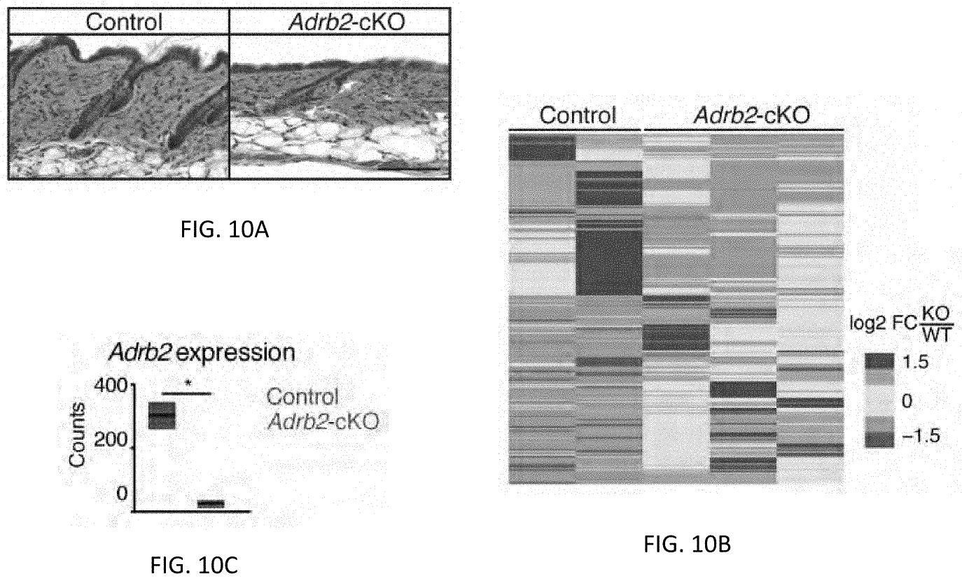

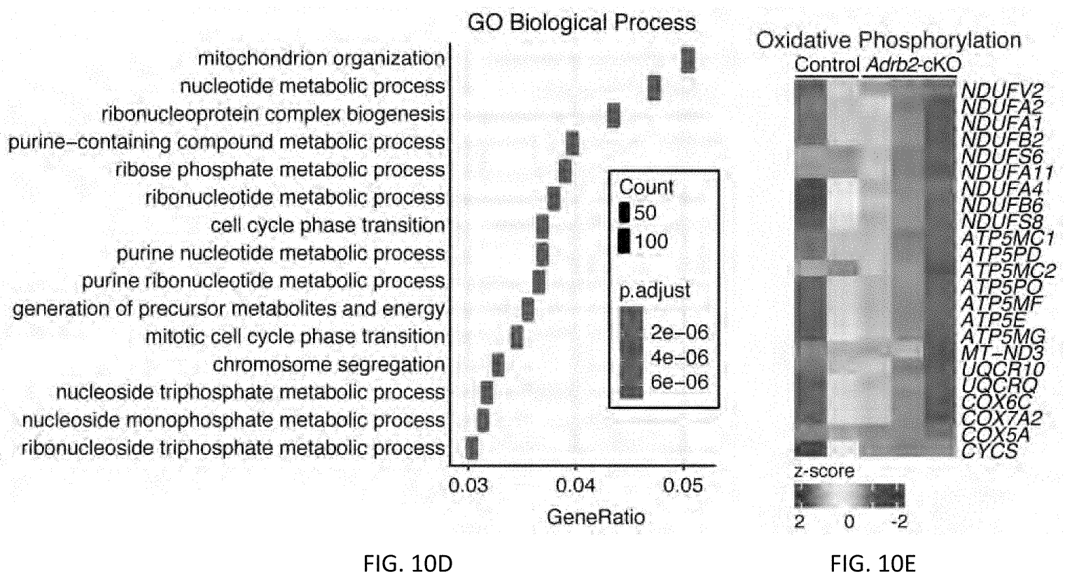

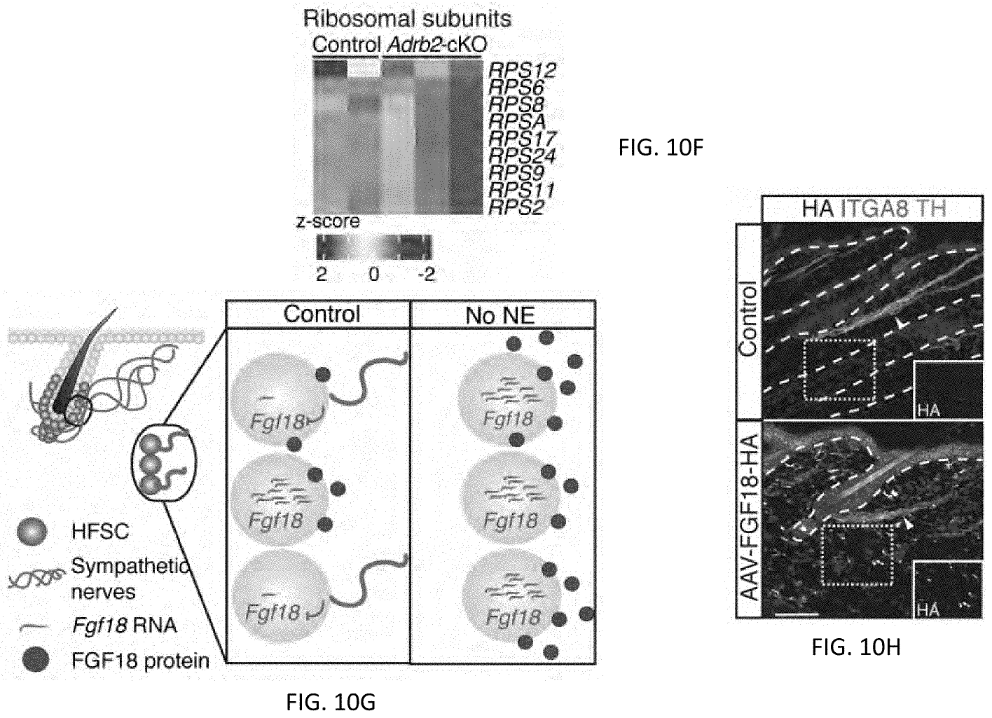

To identify the molecular underpinnings of the delay in hair cycle entry when HFSCs lack Adrb2, RNA-seq analysis was conducted of FACS-purified HFSCs. To pinpoint the functional differences that drive changes, Adrb2 was deleted in telogen and isolated HFSCs when both the control and Adrb2-depleted hair follicles were still in telogen, as confirmed by histological analyses (FIGS. 3A-3B and FIG. 10A). Principal component analysis showed that replicates clustered according to genotypes (FIG. 3C and FIG. 10B). RNA-seq confirmed that Adrb2 is efficiently depleted in HFSCs (FIG. 10C). Ingenuity pathway analysis (IPA) and Gene Ontology (GO) enrichment analysis revealed that cell cycle related categories (including cell division machinery, cell cycle control, and cell cycle checkpoint) were featured as some of the most significantly down-regulated changes in Adrb2-depleted HFSCs (FIGS. 3D-3E and FIG. 10D). These results suggest that even at telogen, Adrb2-depleted HFSCs have already down-regulated cell cycle machinery. Moreover, genes related to oxidative phosphorylation, mitochondria function, and ribosomal components were also down-regulated (FIGS. 10D-10F). Similar categories were featured as hallmarks that distinguish quiescent neural stem cells and muscle stem cells that are more dormant from those that are primed for activation (Llorens-Bobadilla et al., 2015; Rodgers et al., 2014; van Velthoven et al., 2017).

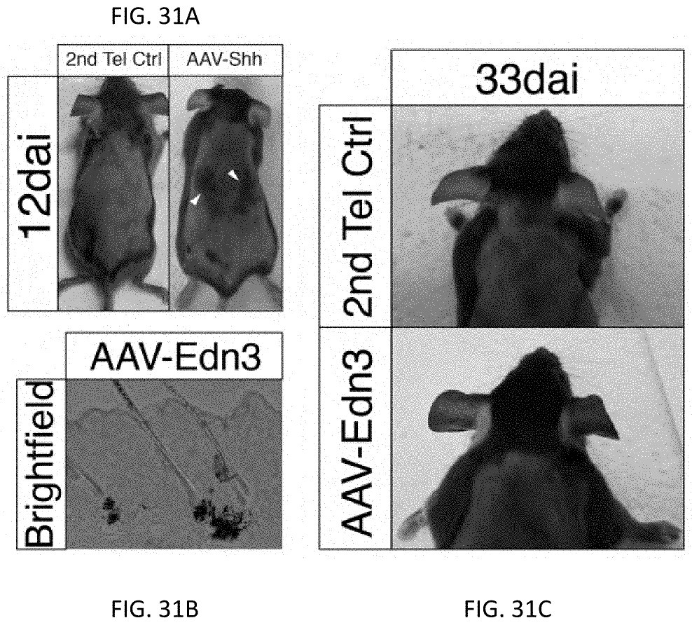

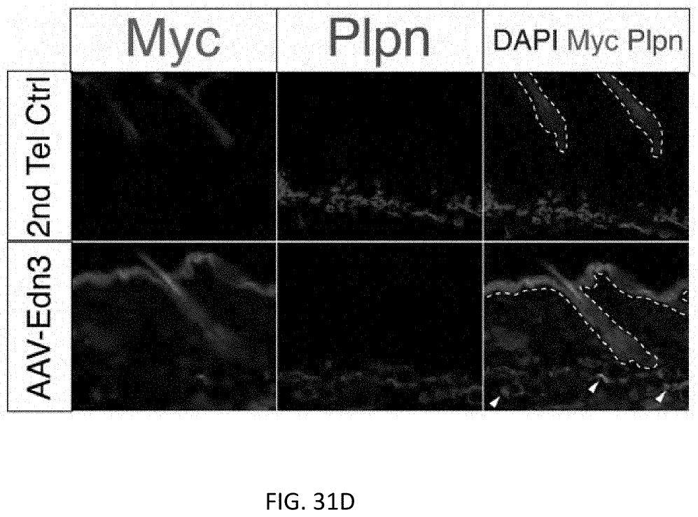

The transcriptomic data also showed several genes known to regulate HFSC quiescence become up-regulated in HFSCs upon Adrb2 depletion (FIG. 3F), including the transcription factor Foxp1 and its downstream target Fgf18 (Hsu et al., 2011; Kimura-Ueki et al., 2012; Leishman et al., 2013) (FIG. 3G). The up-regulation of Fgf18 in Adrb2-depleted HFSCs is particularly notable. HFSCs are located in the outer bulge layer that is innervated by the sympathetic nerve, adjacent to an inner K6+ differentiated bulge layer that is not innervated (FIG. 3H, see also innervation analyses below). In wild-type mice, Fgf18 is highly expressed in the inner K6+ bulge but lower in HFSCs (Hsu et al., 2011). By contrast, in Adrb2-cKO mice, Fgf18 becomes up-regulated in HFSCs as well, as verified by in situ hybridization (FIG. 31). These data suggest that sympathetic innervation keeps Fgf18 levels low at the outer bulge layer where HFSCs reside (FIG. 31 and FIG. 10G). Moreover, overexpression of Fgf18 through injection of Adeno-Associated Viruses (AAVs) (Goldstein et al., 2019) expressing Fgf18 under the control of a CAG promoter suppressed anagen entry (FIG. 3J and FIG. 10H). Together, the data show that upon Adrb2 deletion, HFSCs enter a deep quiescent state governed in part by up-regulation of the Foxpl-Fgf18 axis. These findings link a quiescence pathway with an upstream neuronal signal.

### The Sympathetic Nerve Wraps Around HFSCs

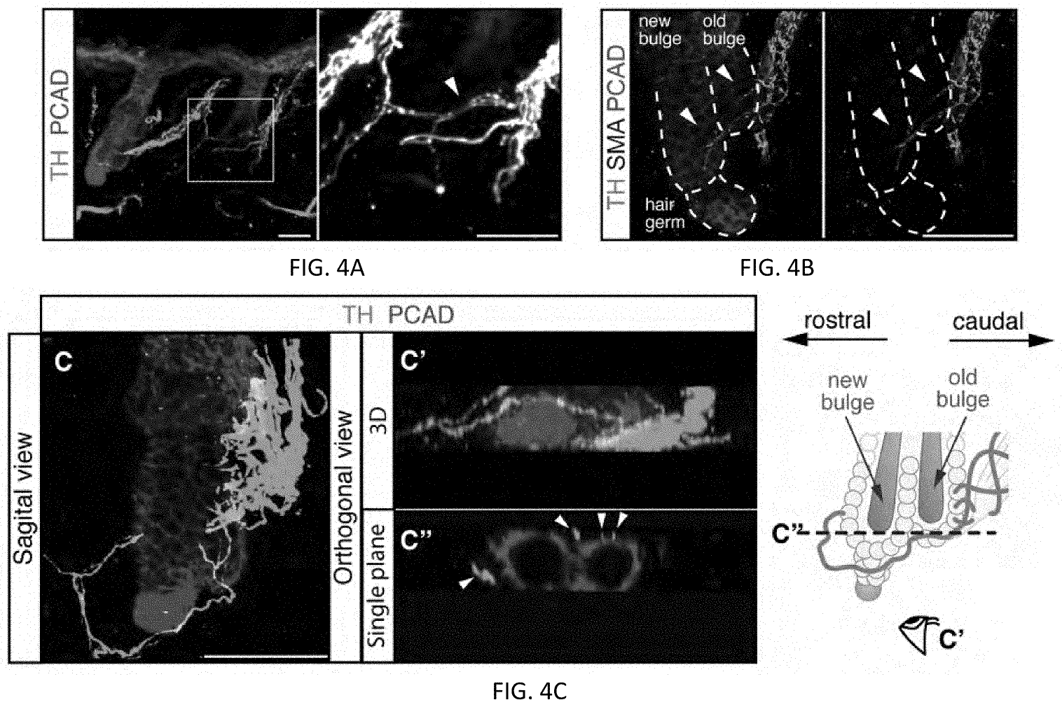

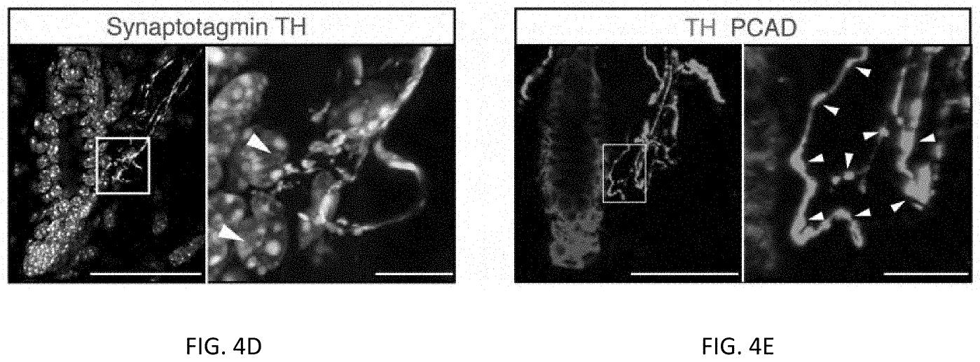

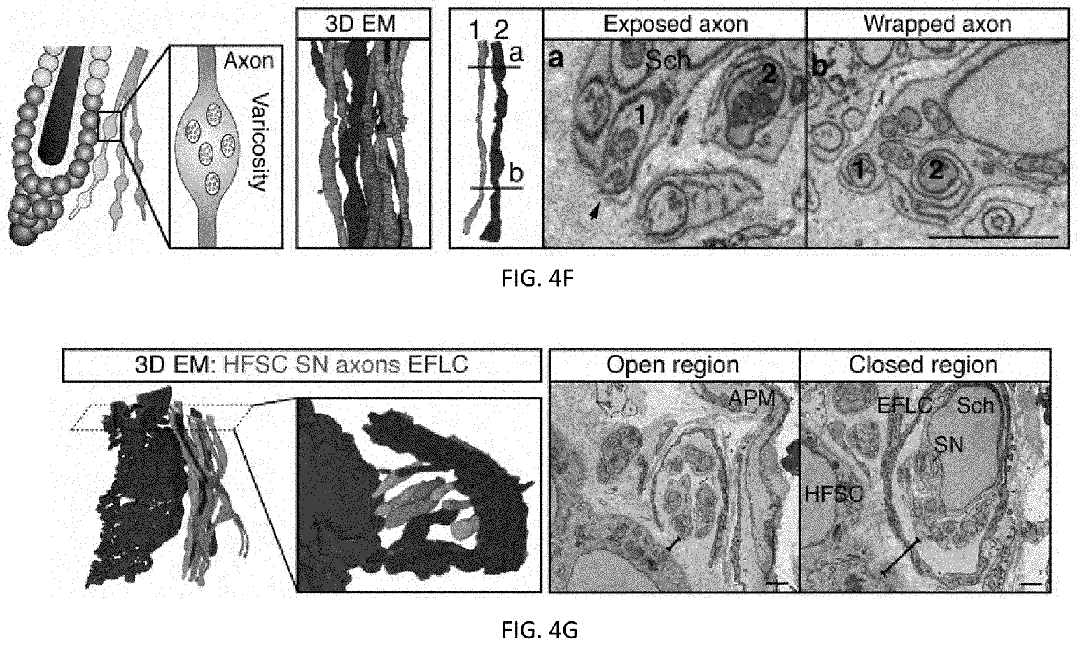

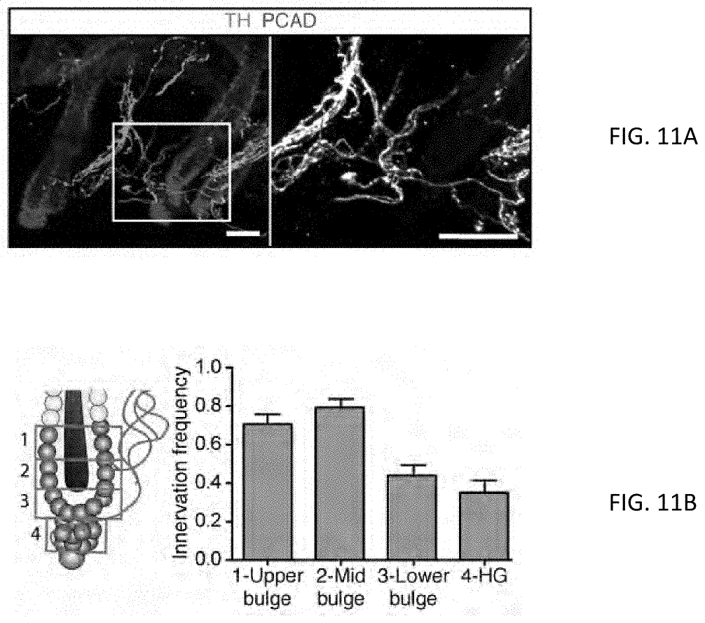

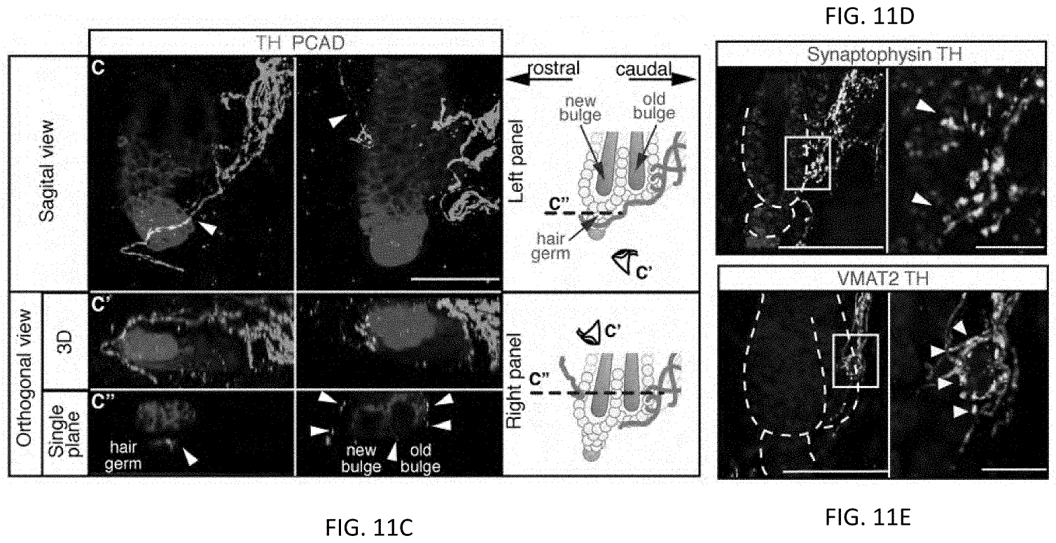

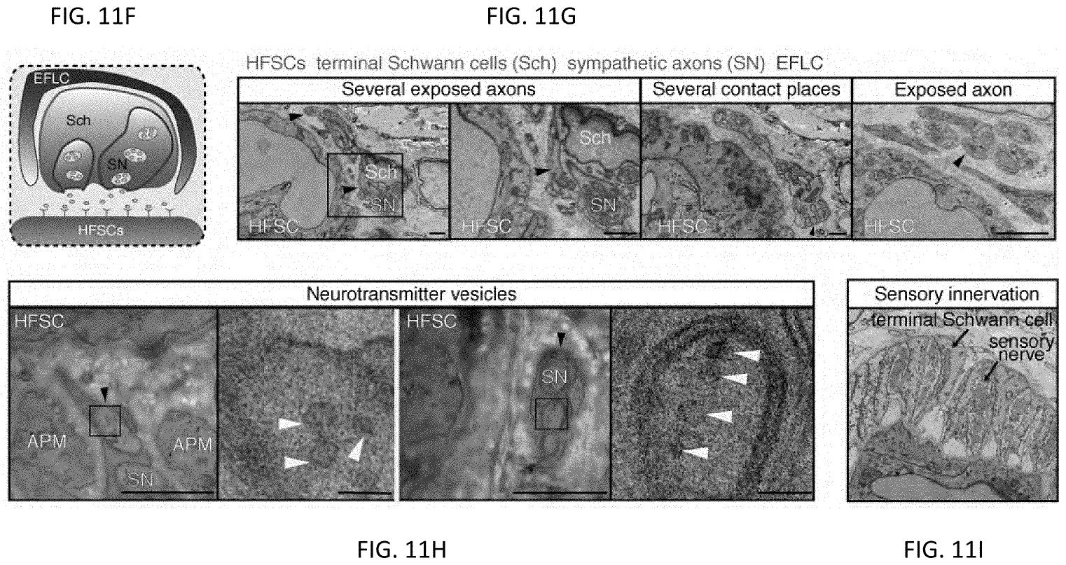

The sensitivity of HFSCs to basal levels of sympathetic nerve activity is notable given that the sympathetic nerve regulates other stem cells either indirectly via the niche or only upon hyperactivation (Katayama et al., 2006; Lucas et al., 2013; Maryanovich et al., 2018; Zhang et al., 2020). It was therefore sought to elucidate the cellular basis of the sympathetic nerve-HFSC interaction that might account for the sensitivity of HFSCs to low levels of norepinephrine. 3-D reconstructed images of thick skin sections (100 μm) revealed that sympathetic nerves form an elaborate neuronal network in the skin (FIG. 4A and FIG. 11A). Consistent with previous findings, it was found that each APM intermingled with dense sympathetic nerve bundles, forming the cellular basis of piloerection (Botchkarev et al., 1999; Furlan et al., 2016) (FIG. 4B). Interestingly, many sympathetic nerve fibers extended beyond the APMs and approached the HFSCs located at different positions throughout the outer bulge and the hair germ (FIGS. 4B-4C and FIGS. 11B-11C). The interaction between sympathetic nerves and HFSCs was not restricted to the sites where APMs attach to the hair follicle (the caudal side). In the 2^{nd} telogen when a new bulge and hair germ form at the rostral side of the APMs, sympathetic fibers innervating both the old and new bulge were observed (FIG. 4C and FIG. 11C). HFSCs were often innervated by sympathetic nerve fibers that branch out from the dense sympathetic bundles along APMs (FIG. 4C and FIG. 11C, left). In some cases, sympathetic nerves approached the new bulge and hair germ from the rostral side, branching from connected bridges linking each sympathetic nerve bundle (FIG. 4C and FIG. 11C, right). 3D-reconstructed images confirmed that sympathetic innervations wrap around both the old and new bulge (FIG. 4C′ and FIG. 11C′). Orthogonal sections showed that sympathetic nerves form multiple contact points with HFSCs located at the old bulge, new bulge, or hair germ (FIG. 4C″ and FIG. 11C″). Collectively, these data suggest that sympathetic nerves innervate not just the APMs but also the HFSCs located throughout the outer bulge and hair germ.

Sympathetic Nerves Form Synapse-Like Connections with HFSCs

Sympathetic nerves innervate smooth muscles or glands to exert their functions through synapses (also known as neuroeffector junctions), allowing efficient activation of intended targets. Epithelial cells like HFSCs are not conventional synaptic targets, but the proximity between nerve endings and HFSCs and the sensitivity of HFSCs to low levels of norepinephrine, prompted the examination of whether sympathetic nerves might form synapse-like connections with HFSCs. Immunofluorescent staining showed that sympathetic nerve fibers co-localize with the pre-synaptic markers synaptotagmin and synaptophysin, (FIG. 4D and FIG. 11D), as well as VMAT2 (Vesicular monoamine transporter 2, marking norepinephrine-containing synaptic vesicles, FIG. 11E), when approaching HFSCs, suggesting that these are terminal axons with norepinephrine vesicles reaching their targets. Furthermore, when sympathetic axons approached HFSCs, swellings of the axon that resemble axonal varicosities (or boutons), structures at the synaptic terminals where neurotransmitters are stored, were identified (FIG. 4E). To examine the interactions between nerves and HFSCs, serial block face scanning EM was conducted (Swanson and Lichtman, 2016). 3D-EM reconstructions confirmed the presence of axonal varicosities around HFSCs (FIG. 4F). EM data showed that the sympathetic fibers were wrapped by Schwann cells. These wrapped nerve fibers were then bundled and enclosed by the endoneurium composed of specialized fibroblasts and collagen. As the sympathetic nerve bundle approached HFSCs, the endoneurium opened only on the side that faces HFSCs, exposing nerve fibers to HFSCs (FIG. 4G and FIG. 11F). This opening likely facilitates the diffusion of neurotransmitters toward HFSCs, as the endoneurium may blunt their transmission. Exposed axons were also observed without Schwann cell wrappings at the side where the sympathetic fibers face HFSCs, which may further enhance the transmission of neurotransmitters to HFSCs (FIG. 4F and FIGS. 11G-11H). Moreover, vesicles and mitochondria (crucial for synaptic transmissions) were observed when these exposed axons approach HFSCs (FIG. 4F and FIG. 11H). These features are morphologically distinct from sensory nerves, which are encased by terminal Schwann cells (Li and Ginty, 2014) (FIG. 11I). These cellular characteristics suggest that sympathetic nerves form synapse-like connections with HFSCs, reminiscent of those found in autonomic neuromuscular junctions or parasympathetic innervation at salivary glands (Burnstock, 2008; Sheu et al., 2017).

### The Sympathetic Nerve-HFSC Interaction is Maintained by APMs

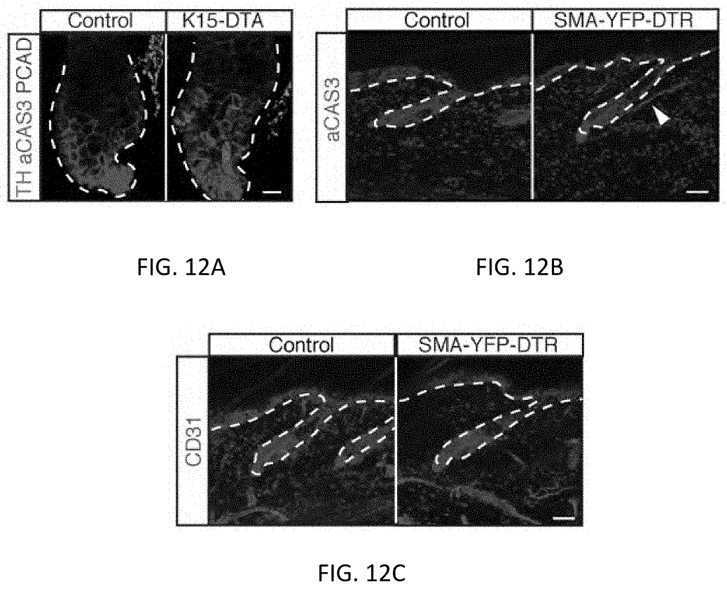

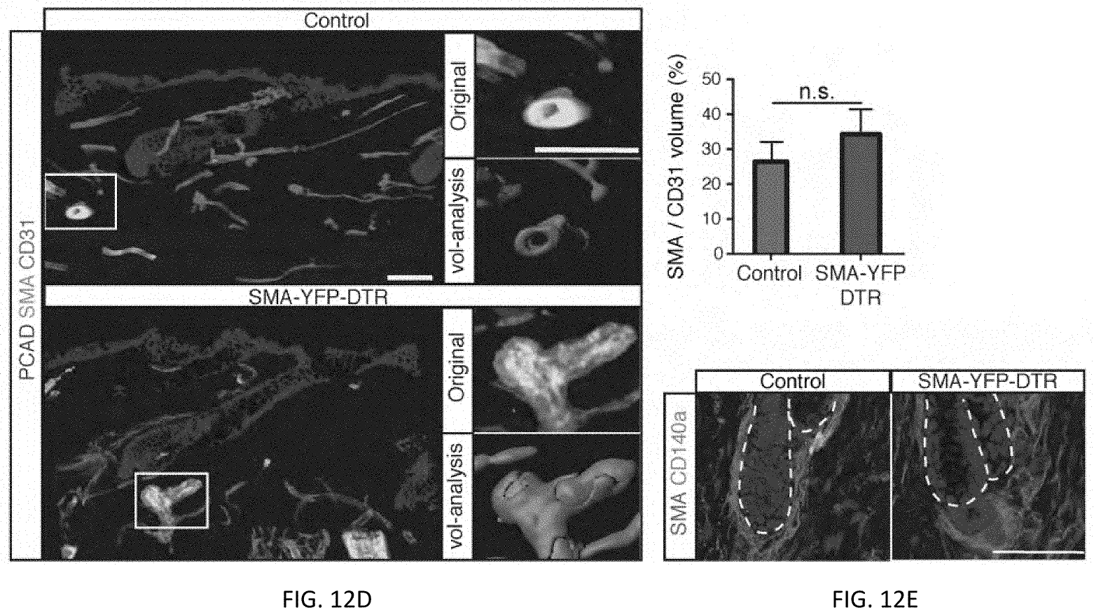

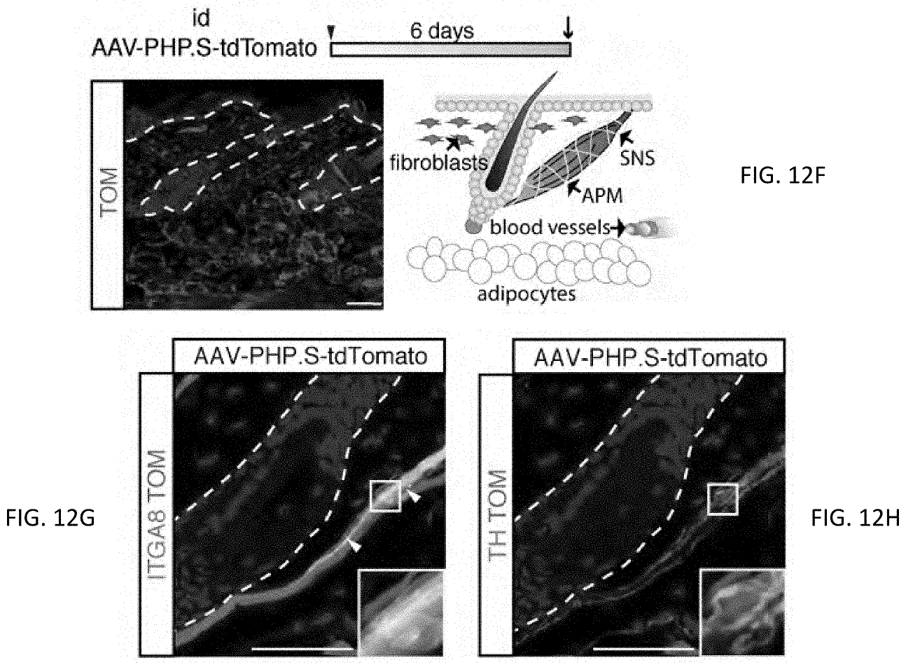

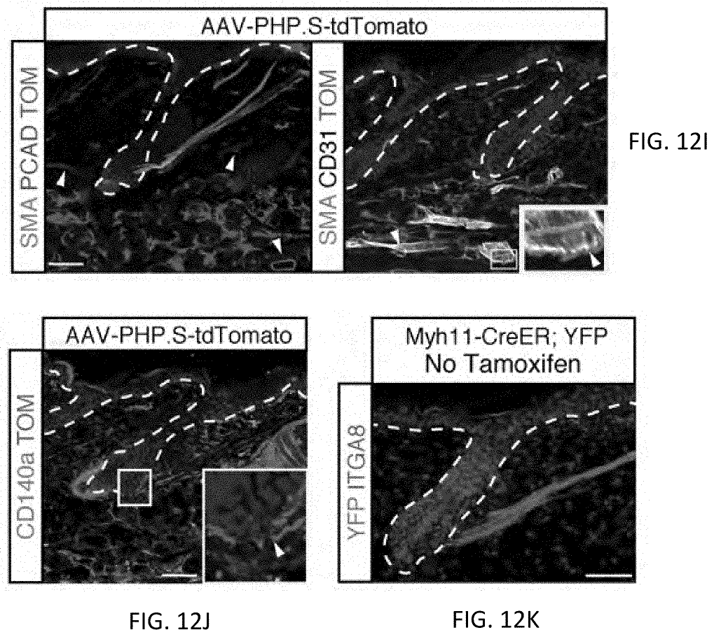

How these nerve-stem cell interactions are maintained in the skin, where dynamic changes occur in both the epithelium and mesenchyme, were explored. It was first tested if HFSCs are responsible for maintaining these nerve-stem cell interactions. However, upon HFSC ablation, sympathetic innervation towards HFSCs was still detected, suggesting that HFSCs are not essential in maintaining these nerve-HFSC interactions (FIG. 12A).

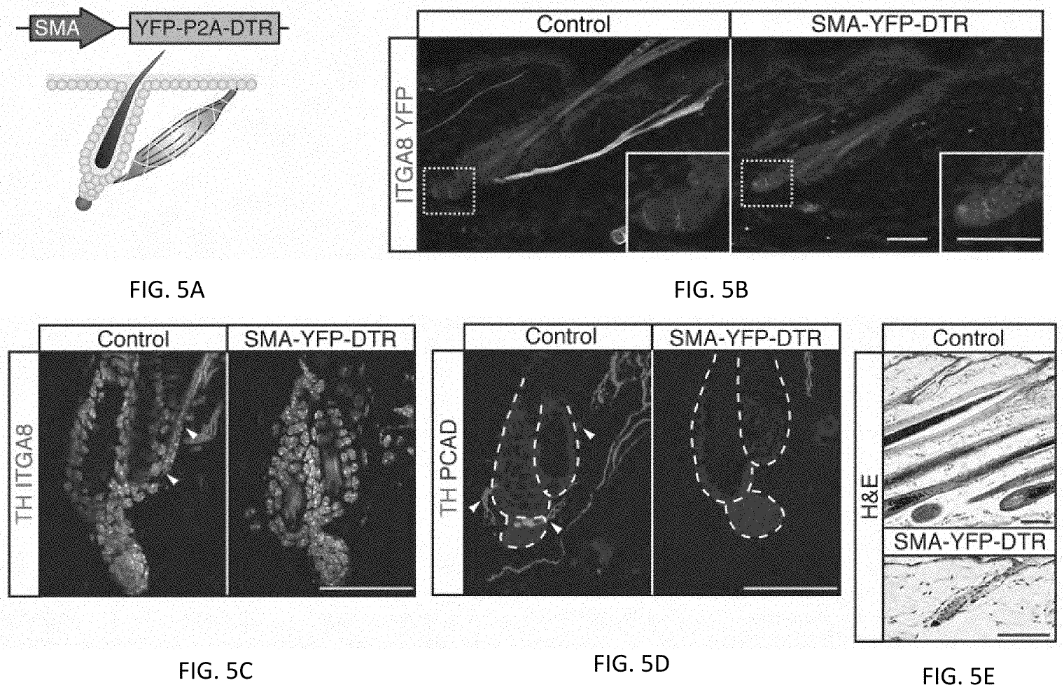

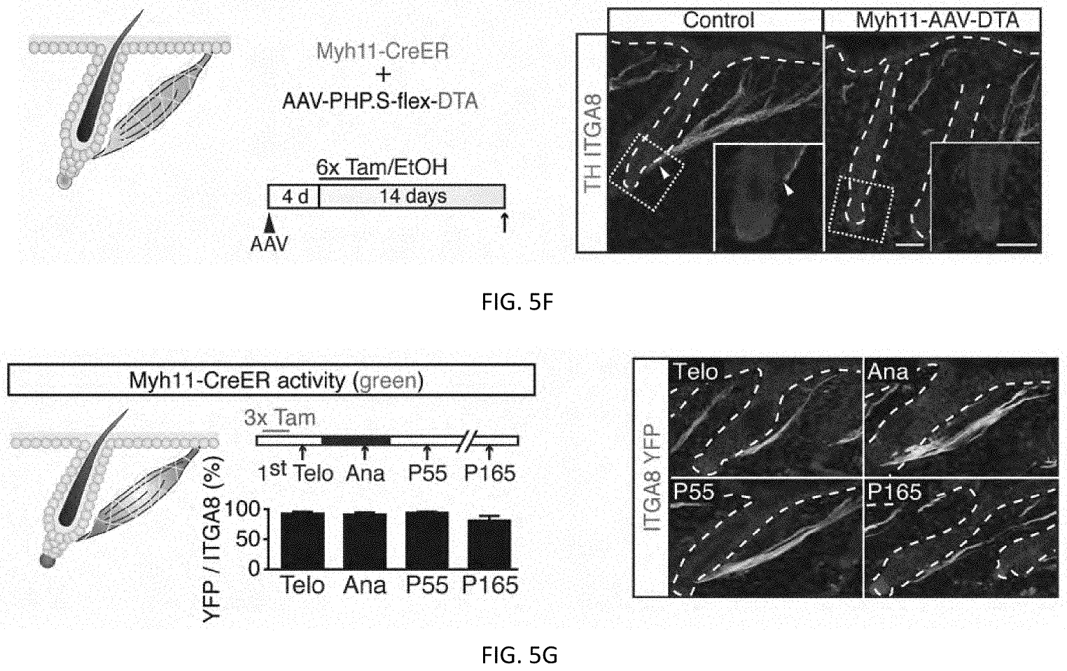

Given that sympathetic nerve fibers were intertwined with APMs, it was next sought to determine if APMs are essential for maintaining sympathetic innervation to HFSCs. To this end, a transgenic mouse model was generated in which both the YFP reporter and the diphtheria toxin receptor (DTR) are expressed under the smooth muscle actin (SMA) promoter (SMA-YFP-DTR mouse, FIG. 5A). YFP staining confirmed that the SMA promoter was active in the APMs but not the dermal sheath in telogen (FIG. 5B, left panel). When diphtheria toxin (DT) was injected intradermally to the SMA-YFP-DTR mice in telogen, active Caspase-3 staining was prominent in APMs, indicating APMs were effectively ablated (FIG. 12B). By contrast, other cell types including dermal papilla, dermal sheath, capillaries surrounding HFSCs, and smooth muscle cells in the subcutaneous vessels remained largely unaffected (FIG. 5B and FIGS. 12C-12E). These data confirm that the SMA-YFP-DTR mouse coupled with intradermal DT injection preferentially ablates APMs. When sympathetic innervation was examined in these APM-ablated mice, it was found that the sympathetic innervation to HFSCs was lost concomitantly following APM ablation (FIGS. 5C-5D). Collectively, these data show that APMs are essential for maintaining the sympathetic innervation to HFSCs. Similar to sympathectomized mice, these APM-ablated mice also displayed a delay in anagen entry (FIG. 5E).

To further confirm the role of APMs in maintaining sympathetic innervation to HFSCs, another method to specifically ablate APMs was established. Intradermal injection of AAV-PHP.S infected APMs and some fibroblasts, but not blood vessels, dermal sheath, sympathetic nerves, or smooth muscles in the subcutaneous vessels (FIGS. 12F-12J). Intradermal injection of a Cre inducible DTA construct (Wu et al., 2014) supplied by AAV-PHP.S into Myh11 -CreER mice allowed for the achievement of specific APM ablation, as APMs are the only cells in the body that carry both CreER and flex-DTA (FIG. 5F). Consistent with the SMA-YFP-DTR model, ablation of APMs leads to loss of sympathetic innervation to HFSCs in Myh11 -CreER; AAV-flex-DTA mice (FIG. 5F). Together, these data establish a crucial role of APMs in maintaining the sympathetic innervation to HFSCs.

Many epidermal and dermal cell types in the skin undergo substantial turnover (Driskell et al., 2013; Heitman et al., 2020; Hsu et al., 2014a; Rivera-Gonzalez et al., 2016; Rompolas et al., 2016; Sada et al., 2016; Zhang et al., 2016), which poses challenges to the maintenance of constant innervation. To determine if APMs also undergo turnover, lineage-tracing experiments were conducted. APMs were labeled at the 1st telogen in Myh11 -CreER; Rosa-lsl-YFP mice. Three days after tamoxifen treatment, the majority of the APM fibers became YFP positive (FIG. 5G). The percentage of labeled APMs did not change over several rounds of hair cycles over a 5-month period. Without tamoxifen, the Myh11-CreER showed minimal leakiness (FIG. 12K). These data suggest that APMs do not undergo major turnover. In this sense, APMs serve as a critical structural support to which sympathetic nerves can remain anchored while both the epithelial and mesenchymal compartments undergo periodical remodeling.

### Cold Triggers Both Piloerection and Hair Growth

The data established that APMs and sympathetic innervation form a dual component niche to regulate HFSC activity. The sympathetic nerve secretes norepinephrine to modulate HFSC activity directly, while APMs maintain sympathetic nerve-HFSC interactions. One known process that requires the concerted action of this tri-lineage unit is piloerection. Given the findings, it was predicted that the elevated sympathetic nerve activity in response to cold may not only induce goosebumps, but might also promote HFSC activation.

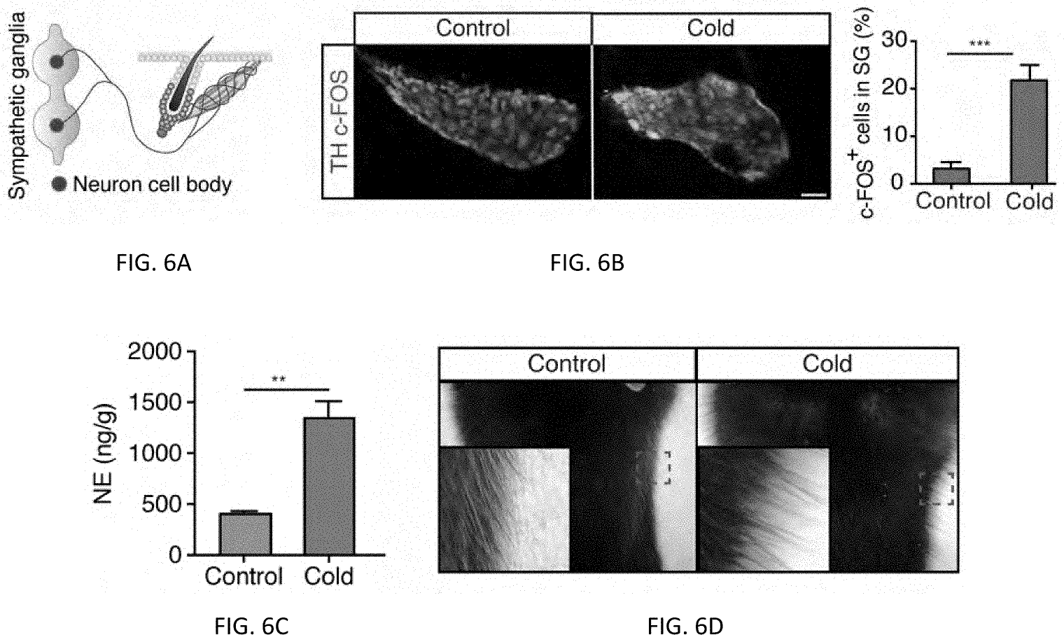

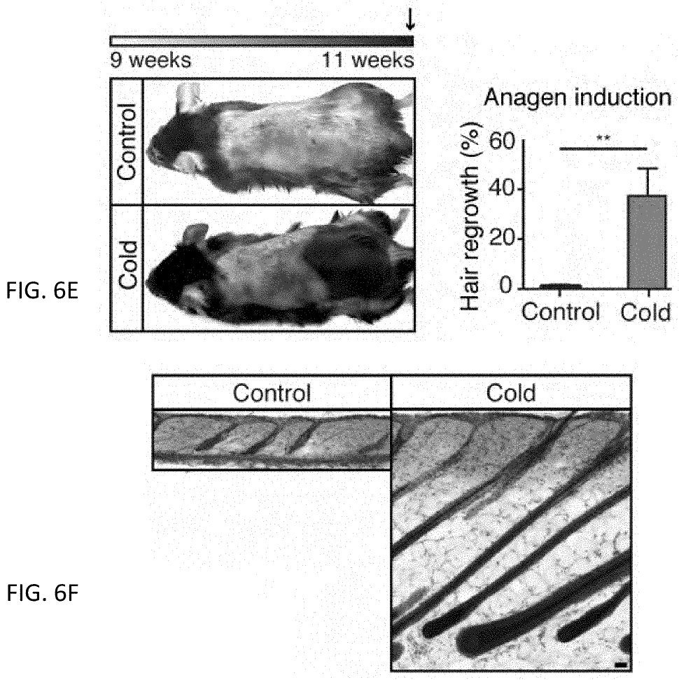

To explore this idea, sex-matched, age-matched telogen mice were compared under cold vs. thermoneutral conditions. Cold exposure indeed enhanced sympathetic nerve activity, as evidenced by elevated c-FOS expression in the sympathetic ganglia (where cell bodies of sympathetic neurons reside) of mice exposed to cold (FIGS. 6A-6B). In agreement with this, the level of norepinephrine was also up-regulated in the skin upon cold stimulation (FIG. 6C). Mice displayed the classical goosebumps reaction in response to cold (FIG. 6D). Moreover, mice exposed to cold entered anagen precociously to produce new hairs within less than 2 weeks (FIGS. 6E-6F). These data demonstrate that temperature changes trigger two reactions—erection of hairs to trap air for thermoregulation and acceleration of HFSC activation to promote the production of a new hair coat.

SHH Secreted from the Developing Hair Follicles Regulates APM Formation and Sympathetic Innervation

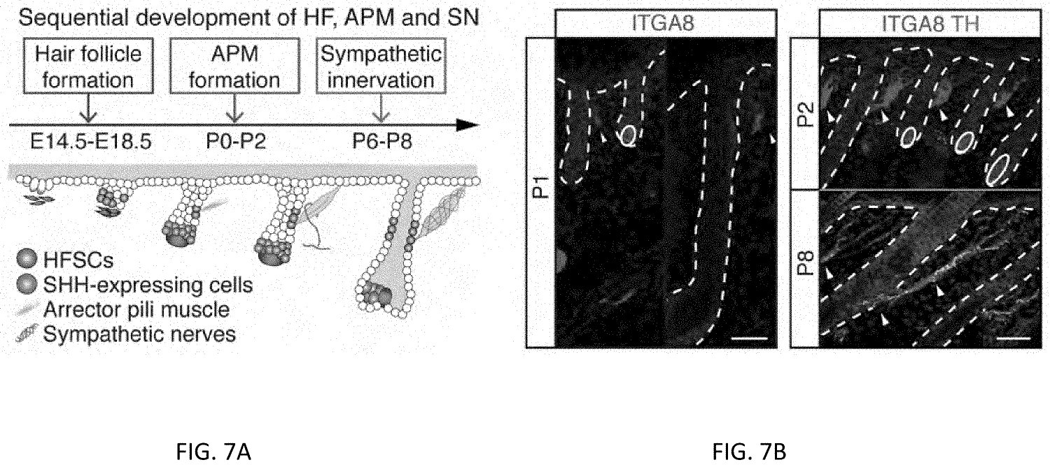

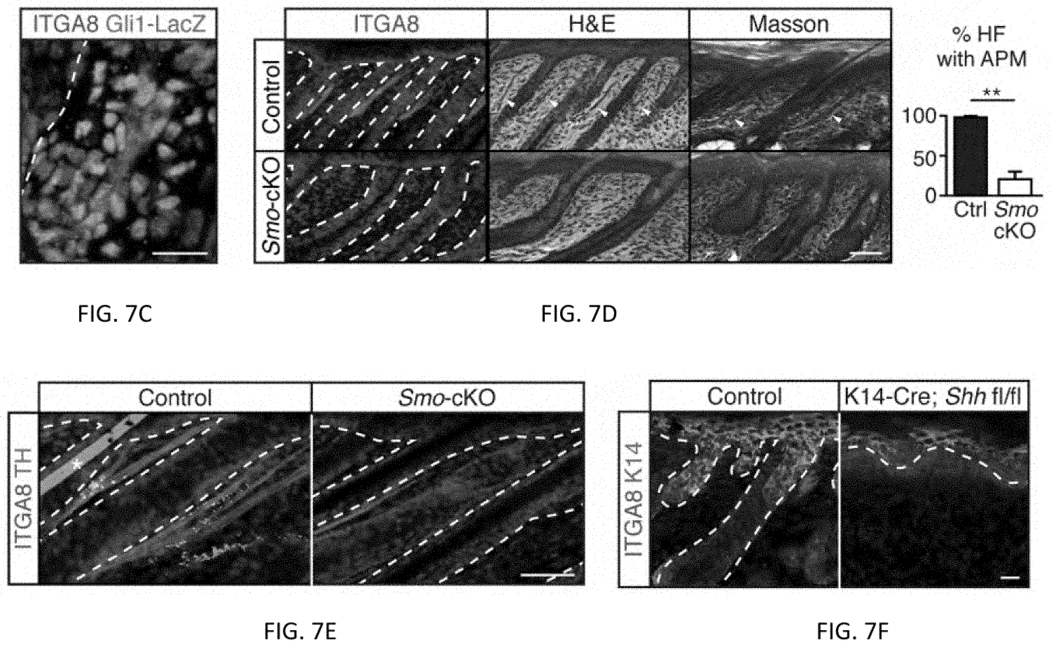

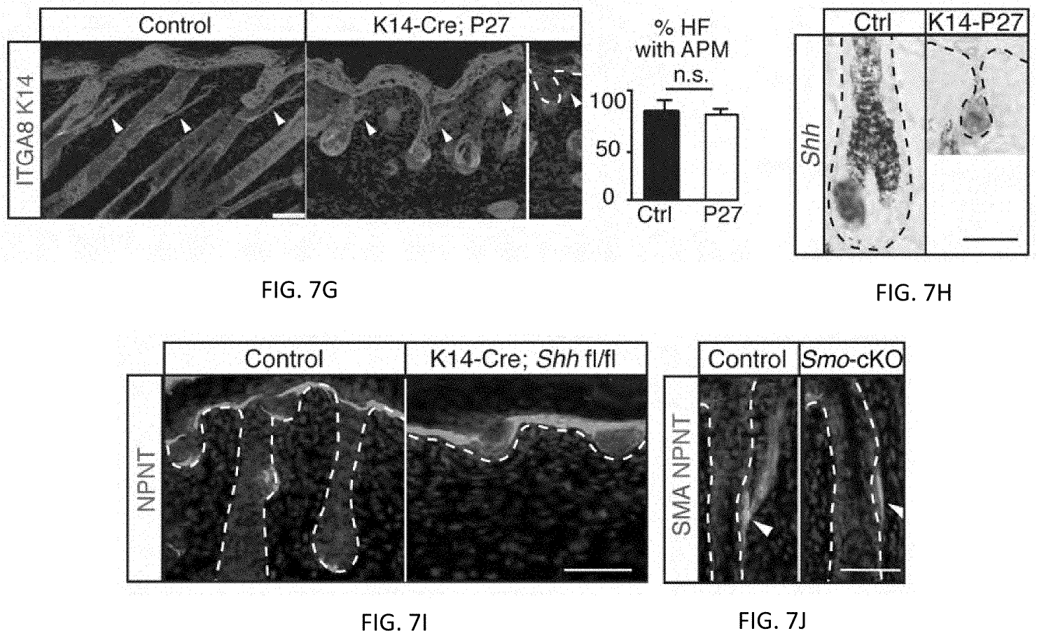

Having established the interconnectivity and function of this tri-lineage unit in tissue regeneration in adults, how this APM-sympathetic nerve niche is established developmentally was explored. First, the developmental timing of APMs and sympathetic innervation to HFSCs was determined. Hair follicles develop in three waves (Andl et al., 2002; Schmidt-Ullrich and Paus, 2005). By postnatal day P1, APMs appeared around the down-growing hair follicles formed during the 1^{st} wave but were absent from the budding hair follicles that just emerged at the 3^{rd} wave. By P2, APMs were found in all hair follicles as they matured. By contrast, sympathetic nerves only began to innervate APMs around P5. By P8, the sympathetic innervation to both APMs and HFSCs became apparent (FIGS. 7A-7B). These results demonstrate the hair follicle is the first tissue to form in this tri-lineage unit, followed by APM, and the sympathetic nerve only innervates HFSCs after APMs form and mature.

Given that the emergence of APMs correlates with the degree of maturation in the hair follicle, it was explored if signals from the hair follicle regulate APM formation. One candidate signal is SHH, a long-range secreted protein from transit-amplifying cells of the developing hair follicle (HF-TACs) (St-Jacques et al., 1998; Zhang and Hsu, 2017). Through SHH, HF-TACs regulate diverse processes including hair follicle downgrowth, dermal adipocyte production, and Merkel cell formation (Chiang et al., 1999; Hsu et al., 2014b; Perdigoto et al., 2016; Woo et al., 2012; Xiao et al., 2016; Zhang et al., 2016). Developing APMs were found to be positive for Glil, a target of Hedgehog (HH) signaling (FIG. 7C).

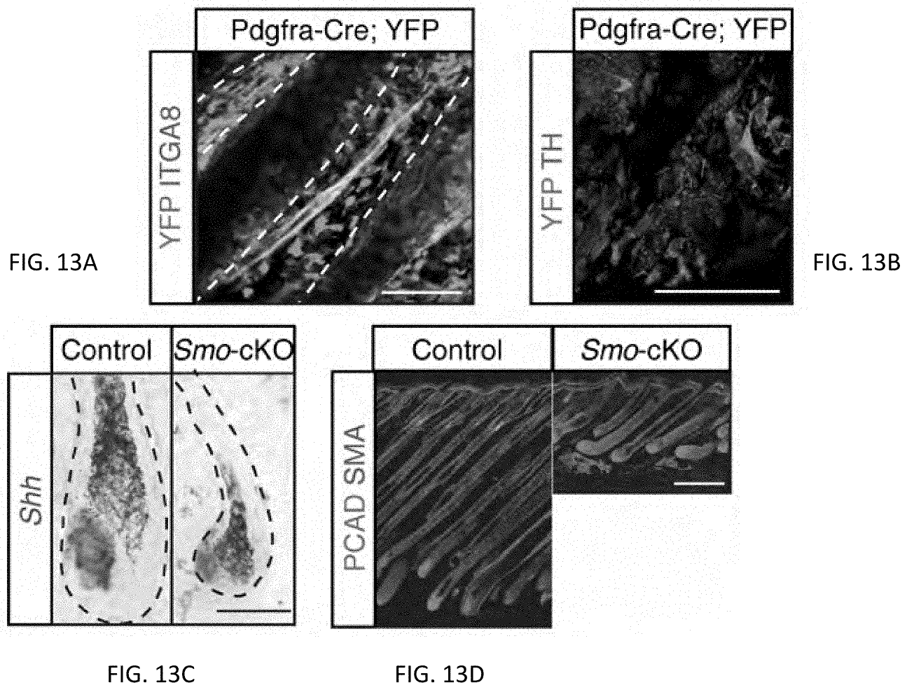

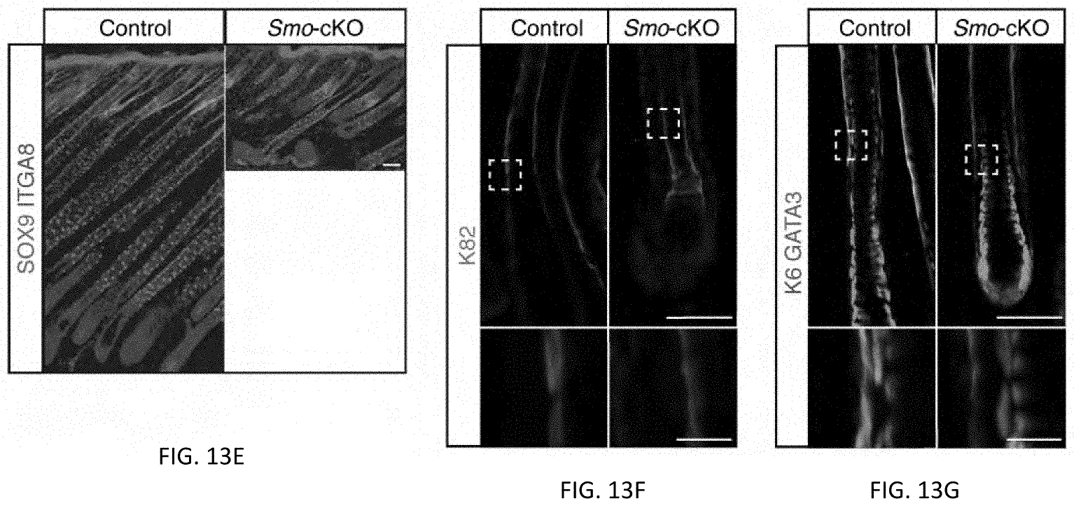

To determine if HH signaling regulates APM formation, Smoothened (Smo, a component required for HH signal transmission) was deleted from the dermis using Pdgfra-Cre. Lineage analysis confirmed that APMs but not sympathetic neurons are derived from PDGFRA positive dermal fibroblasts (Driskell et al., 2013) (FIGS. 13A-13B). Pdgfra-Cre; Smo fl/fl micemice could form HFSCs and differentiated progeny, but lacked APMs, suggesting that HH signaling is required for APM formation (FIG. 7D and FIGS. 13C-13G).

It was then asked if sympathetic innervation to the developing HFSCs requires APMs. Pdgfra-Cre; Smo fl/fl mice not only lacked APMs but also lacked sympathetic innervation to HFSCs (FIG. 7E), suggesting that APMs establish the sympathetic innervation to HFSCs during development. This lack of sympathetic innervation was unlikely due to a requirement of Smo in the sympathetic nerve itself, because Pdgfra-Cre was not expressed in sympathetic neurons (FIG. 13B). Collectively, these data suggest that HH signaling regulates the formation of APMs. Once APMs form, they then attract sympathetic innervation to HFSCs.

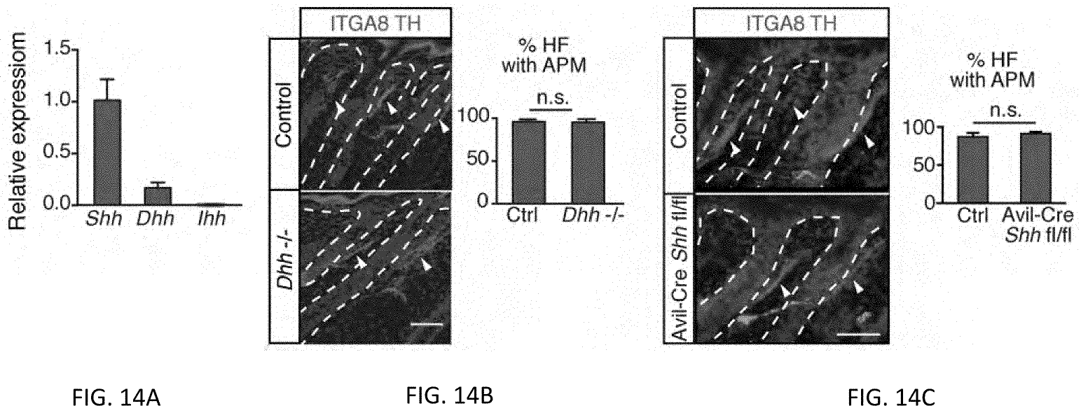

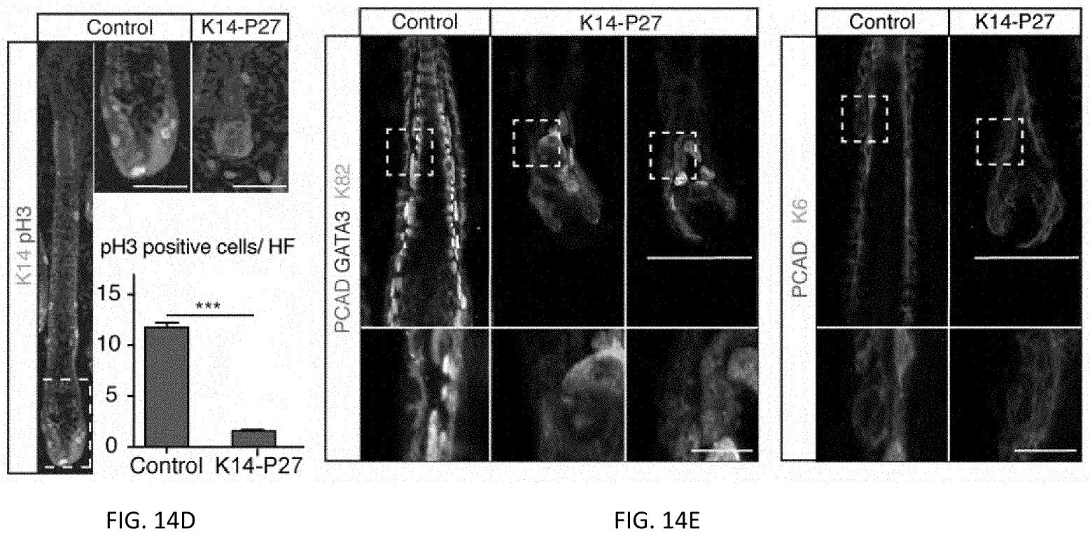

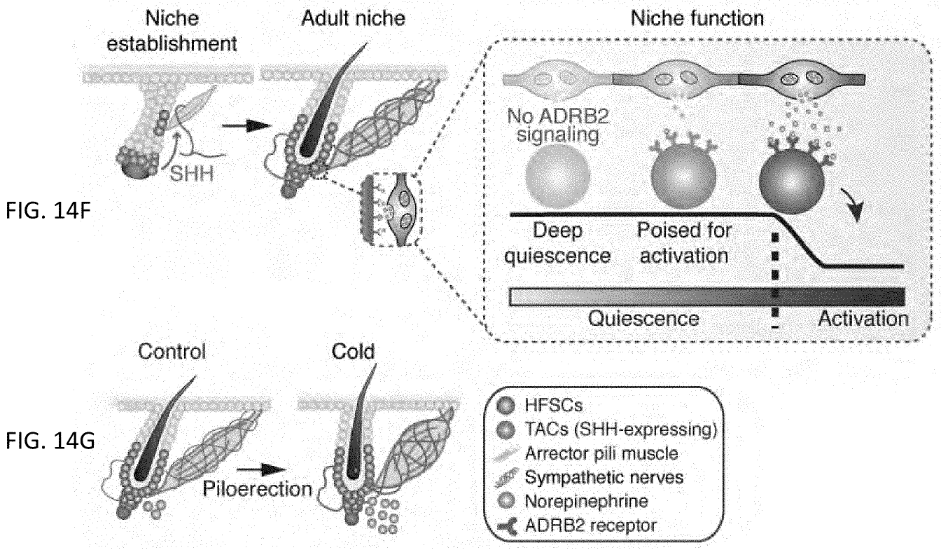

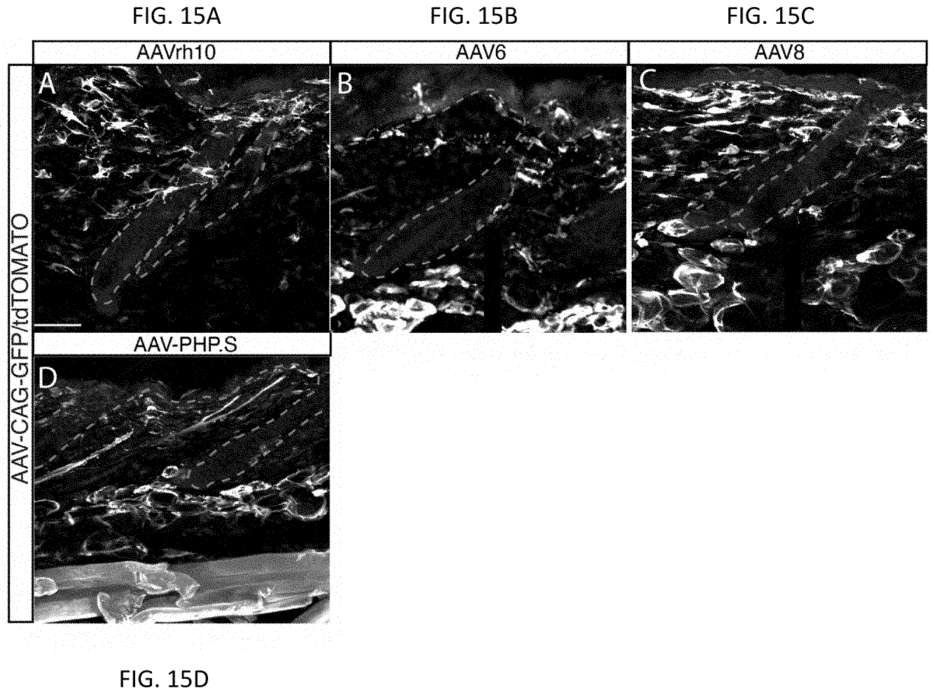

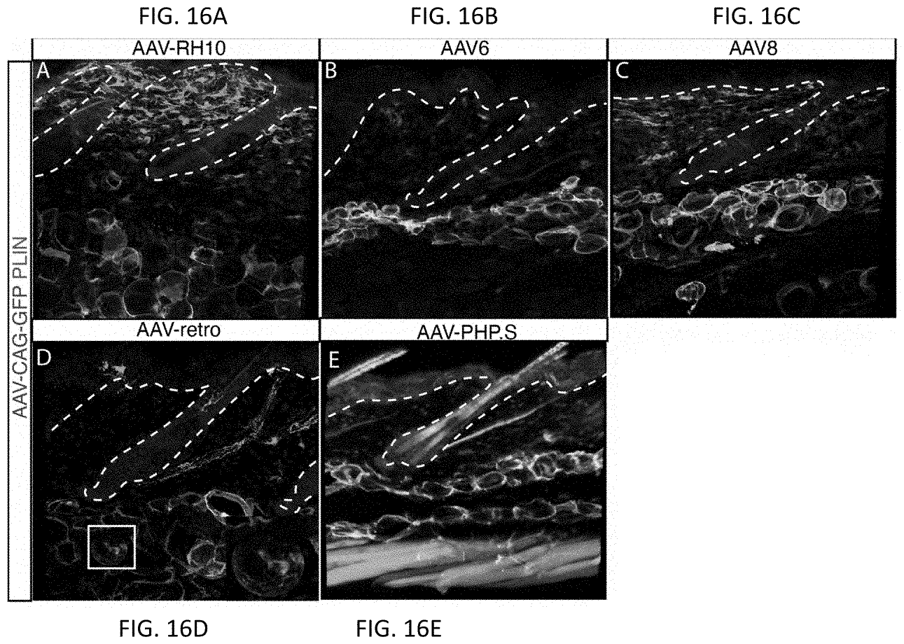

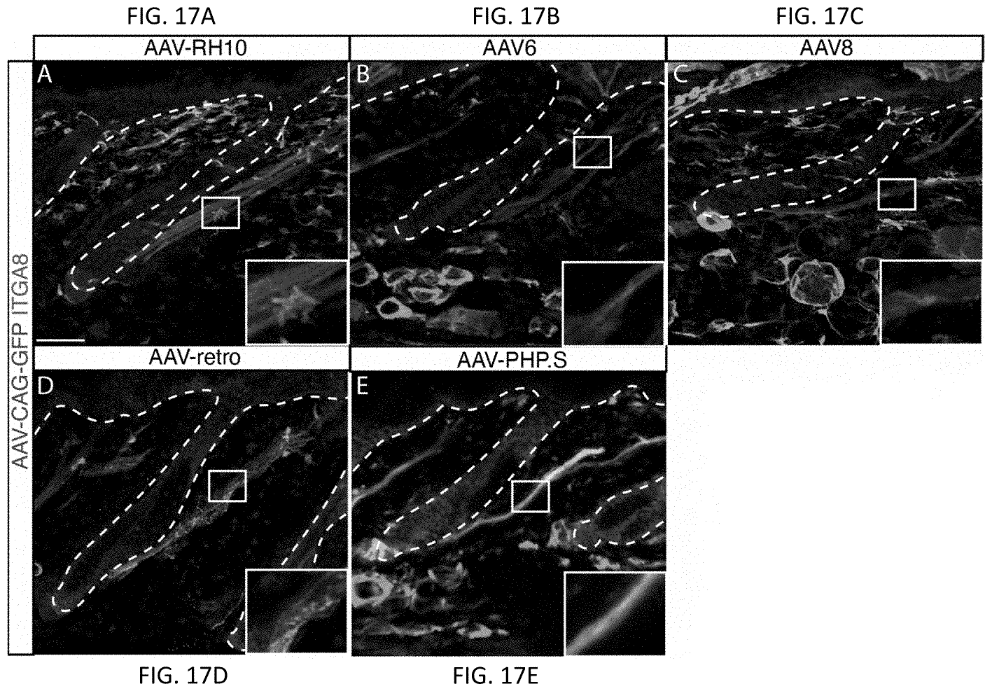

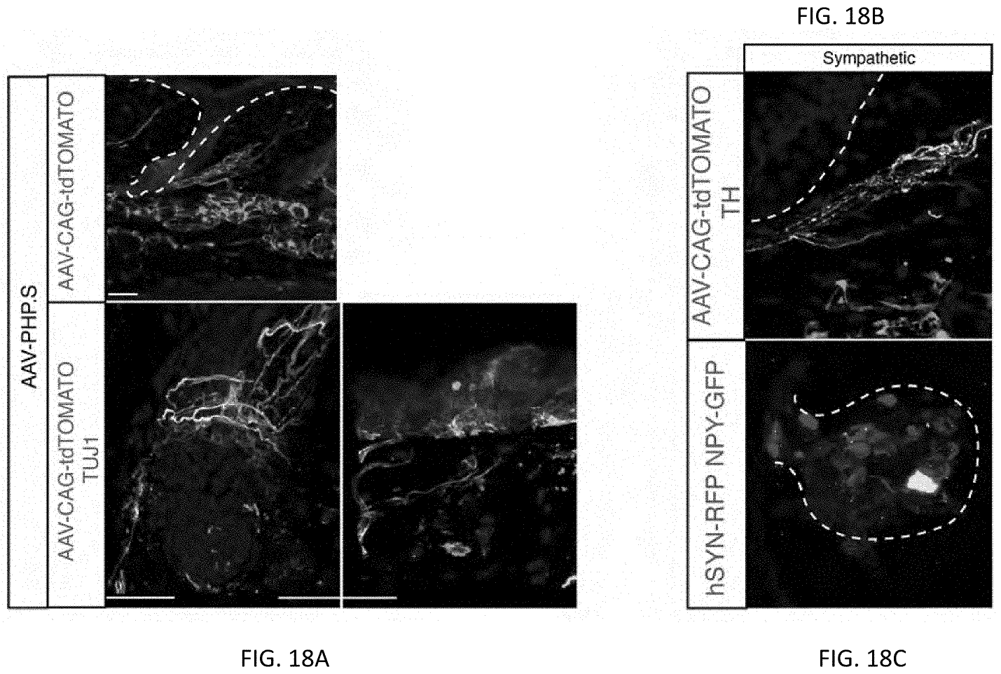

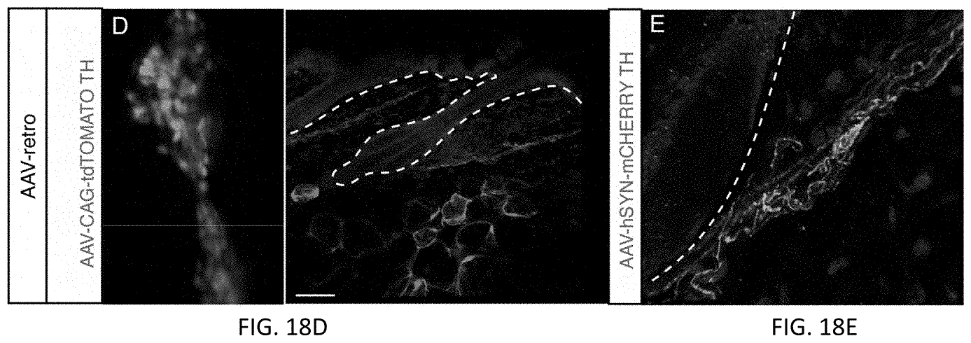

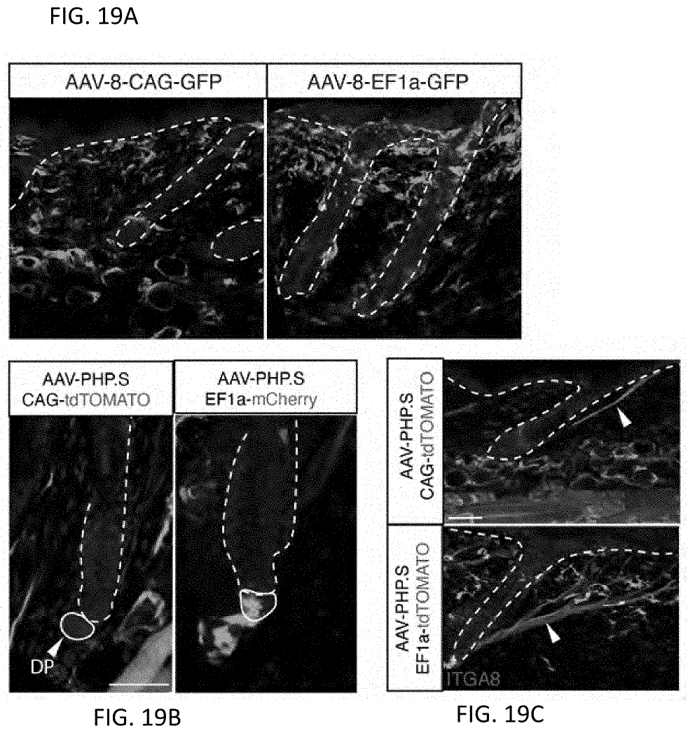

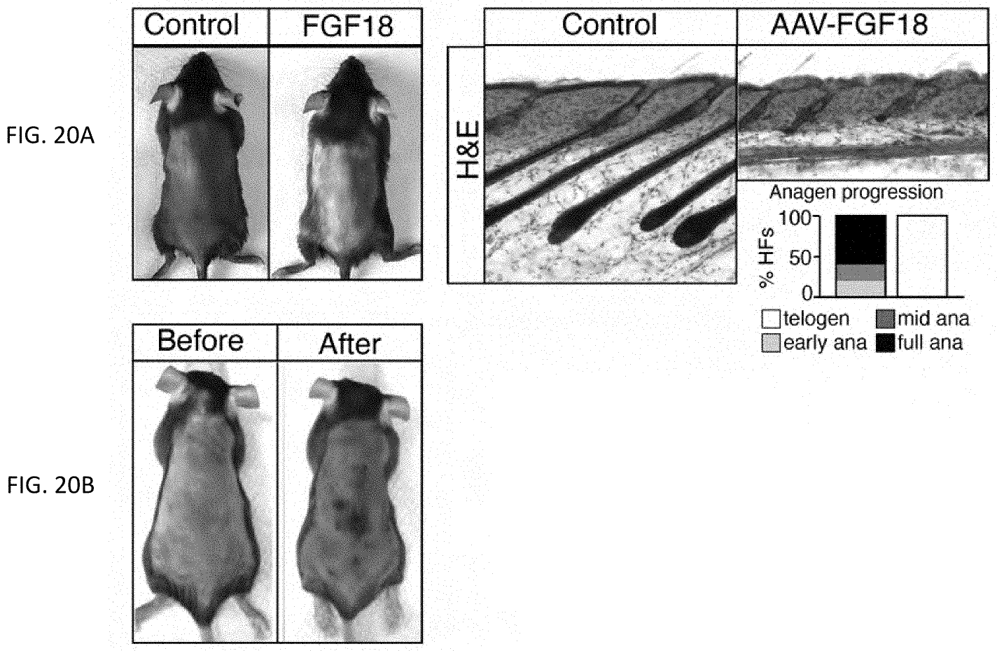

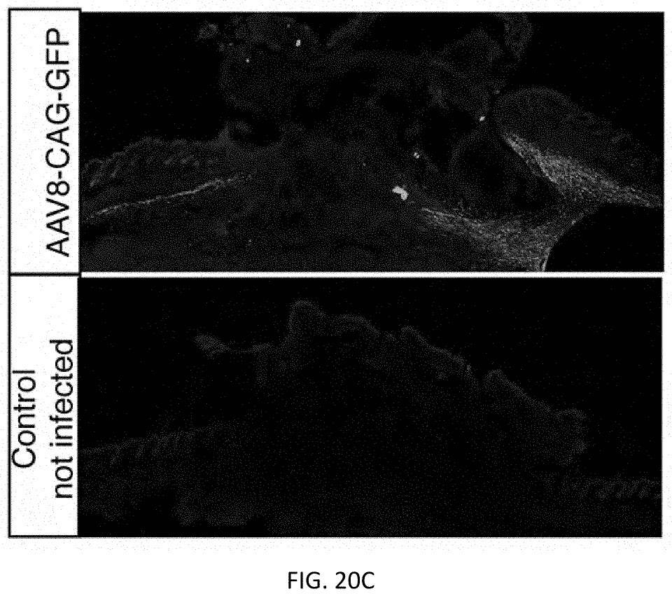

Next, it was aimed to identify the source of HH that regulates APM formation. Ihh is not expressed in the skin (Rezza et al., 2016; Sennett et al., 2015) (FIG. 14A), and Dhh mutants still developed APMs (FIG. 14B). However, when Shh was depleted from developing hair follicles by K14-Cre, APMs failed to form (FIG. 7F). By contrast, APMs remained intact when Shh was depleted from the sensory nerves (FIG. 14C), another source of SHH in the skin (Brownell et al., 2011; Zurborg et al., 2011). These data suggest that SHH from the developing hair follicle drives APM development.

SHH regulates hair follicle downgrowth (Chiang et al., 1999; St-Jacques et al., 1998; Woo et al., 2012). As such, the hair follicle in the K14-Cre; Shh fl/fl skin is defective. To determine if lack of APMs was due to defects in hair follicle growth in the K14-Cre; Shh fl/fl skin, a K14-Cre; Rosa-lsl-rtTA; TetO-P27 model was established to block the downgrowth of hair follicles without affecting Shh expression (FIGS. 7G-7H and FIGS. 14D-14E). APMs were present in K14-Cre; Rosa-lsl-rtTA; TetO-P27 skin, despite severe defects in hair follicle downgrowth and development (FIG. 7G and FIG. 14E). These data suggest that defects in hair follicle development do not cause APM loss as long as Shh is present. APMs attach to the bulge via the integrin Nephronectin, but APMs still form in Nephronectin mutants (Fujiwara et al., 2011). Nephronectin expression also remained intact in both K14-Cre; Shh fl/fl and Pdgfra-Cre; Smo fl/fl skins, suggesting that mechanisms regulating APM formation and APM attachment are distinct (FIGS. 7I-7J). In conclusion, the data demonstrate a reciprocal interaction between the hair follicle and its APM-sympathetic nerve niche at different stages. During development, hair follicles control the formation of APMs that then attract sympathetic innervations. In adults, sympathetic innervations, anchored on APMs, activate HFSCs and promote hair follicle regeneration (FIG. 14F).

### Discussion

### Cell Types Enabling Goosebumps Form a Dual Component Niche for HFSCs

The erection of hairs, feathers, and spines plays a role in thermoregulation, courtship, and aggression, features essential for evolutionary success across the animal kingdom (Darwin, 1872). The anatomical connection between APMs and HFSCs is conserved across mammals, raising the possibility that there might be evolutionary advantages to preserving this anatomical connection beyond goosebumps. It was found that cell types enabling goosebumps form a dual component niche for HFSCs: a supporting component (the APM) and a signaling component (the sympathetic nerve), with the former maintaining the latter. It is possible the APM is evolutionarily conserved due to its indispensable role as a hub to attract and maintain sympathetic innervations in the skin.

Sympathetic neurons differ from other niche cell types for HFSCs (Chen et al., 2020) in that they are both a niche component and a systemic regulator. As a part of the autonomic nervous system, sympathetic innervation provides a direct channel to rapidly transmit systemic changes into local tissue changes. This direct system-to-local connection may allow the activation threshold of HFSCs to vary in response to temperature, circadian rhythm, or physiological changes. In this sense, goosebumps may only be the first line of defense in responding to cold. When cold conditions persist, elevated sympathetic nerve activity allows HFSCs to exit quiescence and initiate hair follicle regeneration to make new hair, coupling stem cell activity and tissue production with outside environmental changes (FIG. 14G).

APMs are often lost in the scalp skin of people with androgenetic alopecia (common baldness) (Torkamani et al., 2014; Yazdabadi et al., 2012). It is possible that in such skin, loss of APMs leads to the loss of sympathetic nerves, making HFSCs more difficult to activate. The results also suggest the potential of using selective (32 agonists to promote HFSC activation.

### The Impact of Sympathetic Nerve Activity on Different Stem Cell Populations

The sympathetic nerve is known to influence melanocyte stem cells (MeSCs), a distinct stem cell population also located around the bulge that regenerates the pigment to color the hair (Zhang et al., 2020). Hyperactivation of sympathetic neurons, as occurs in severe stress, depletes MeSCs, forming the basis for stress-induced hair graying. There are several interesting differences regarding how the sympathetic nerve regulates MeSCs vs. HFSCs. First, HFSCs are more sensitive to low levels of sympathetic nerve activity than MeSCs. HFSCs respond to both basal and modest elevation of sympathetic tone (such as in cold), a characteristic likely facilitated by the synapse-like connections between sympathetic nerve terminals and HFSCs. By contrast, MeSCs are only depleted upon sympathetic nerve hyper-activation. MeSCs are outside of the synaptic transmission range (˜1-2 μm for sympathetic nerve), and are likely influenced by norepinephrine mostly through diffusion, which is only effective at high concentrations. Moreover, whereas HFSCs are positively likely that the sympathetic nerve innervates the hair follicle to regulate HFSCs, while depletion of MeSCs is an undesired side effect when the nerve activity is abnormally high. Future studies are needed to explore how the sympathetic nerve drives different outcomes for distinct stem cells based on differences in the amplitude and duration of nerve activation.

There are also interesting similarities and parallels between the skin and the bone marrow. In both systems, there is a close interplay among stem cells, nerves, and mesenchyme. In the skin, sympathetic nerves regulate HFSCs directly through innervation, and the mesenchymal component (APMs) maintain this nerve-stem cell interaction. In the bone marrow, sympathetic nerves regulate hematopoietic stem cell retention and egression indirectly by regulating Cxcl12 expression in the mesenchyme (Heidt et al., 2014; Katayama et al., 2006).

### Epithelial Stem Cells are an Unconventional Post-Synaptic Target

Neurons regulate excitable targets (e.g. neurons or muscles) through synapses. Here it was shown that sympathetic nerves can also modulate an epithelial stem cell, an unconventional target, through a classical neurotransmitter with synapse-like connections. Neurotransmitters are unstable, so synapse-like structures minimize random diffusion of neurotransmitters and direct them towards the intended targets. Here, the short-range effect of norepinephrine is further propagated by regulating a secreted protein FGF18 that is more stable and has a longer working distance. This allows the nerve signal to extend beyond HFSCs that are innervated to other HFSCs that may not receive direct innervation (FIG. 10G). Such a relay mechanism may be widely applicable when considering how innervations and neurotransmitters can modulate a wide variety of biological processes outside of the nervous system with limited innervation sites.

Sympathetic nerve innervates smooth muscles, glands, and endocrine cells across the body. It was postulated that some cell types adjacent to these conventional nerve targets may also receive direct neuronal input with similar structures and mechanisms as was described here for HFSCs. In particular, epithelial stem cells (for example, those in the airway or gut), which are in close proximity to smooth muscle cells, are prime candidates for this type of regulation. It is also possible that cancer cells hijack similar mechanisms to connect with the nervous system, as solid tumors are often highly innervated (Kamiya et al., 2019; Peterson et al., 2015; Venkataramani et al., 2019; Venkatesh et al., 2019; Zahalka et al., 2017). Collectively, the findings reveal the cellular and molecular mechanisms underlying the interaction between the sympathetic nervous system and an unconventional target, opening up future avenues for investigation into the potentially broad function of such interactions.

### Experimental Model and Subject Details

### Mouse Lines

K15-CrePGR (Morris et al., 2004), K14-Cre (Dassule et al., 2000), Smo fl/fl (Long et al., 2001), Shh fl/fl (Lewis et al., 2001), Rosa-lsl-YFP (Srinivas et al., 2001), Pdgfra-Cre (Roesch et al., 2008), Advillin-Cre (Zurborg et al., 2011), Myh11-CreER (Wirth et al., 2008), Adrb2 fl/fl (Hinoi et al., 2008), GliI-Lacz (Bal et al., 2002), Rosa-lsl-DTA (Voehringer et al., 2008), Rosa-lsl-attenuated DTA (Wu et al., 2006), Dhh −/− (Bitgood et al., 1996), Rosa-rtTA-IRES-EGFP (Rosa-lsl-rtTA) (Belteki et al., 2005), TetO-P27 (Pruitt et al., 2013), and TH-CreER (Abraira et al., 2017) mice were described previously. The SMA-YFP-DTR transgenic mouse line was generated as follows. The plasmid pACTA2-YFP-P2A-DTR was constructed by replacing the CMV promoter region of pcDNA3.1 with the 4-kilobase (kb) fragment of the mouse ACTA2 promoter/intron derived from C57BL/6 genomic DNA. The human HB-EGF cDNA and YFP cDNA were ligated with P2A and cloned into a pcDNA3.1-ACTA2 plasmid. The 6.2-kb MfeI/DraIII fragment from pACTA2-YFP-P2A-DTR was microinjected as a transgene into fertilized mouse eggs (C57BL/6), which were then implanted into pseudo-pregnant female mice (C57BL/6). Integration of the transgene was checked by PCR analysis of DNA extracted from tail tissues. All procedures were performed with animal protocols approved by the Institutional Animal Care and Use Committee at Harvard University, Joslin Diabetes Center, or National Taiwan University. All mice used were specific-pathogen free and housed in individually ventilated cages (max. 5 per cage) under a 12:12 light-dark cycle at 21-25° C. and 30%-75% humidity. Housing and husbandry conditions for cold exposure experiments are described as bellow. Mice were fed ad libitum with rodent diet (LabDiet Prolab Isopro RMH 3000 5P75 or PicoLab Mouse Diet 20 5058) and water. Animal health was monitored daily. Surveillance for infectious agents was performed quarterly. All procedures and treatments are described as in Method Details. None of the mice were involved in any previous procedures prior to the study.

### Method Details

### Cold Exposure

Individually caged C57BL/6J mice (JAX 00064 sex- and age-matched) were housed at an ambient temperature of 5° C. for a period of 2 hours or 2 weeks. Control animals were individually caged and housed at a thermoneutral (30° C.) temperature. Both groups of mice were housed in a controlled environmental diurnal chamber (Caron Products & Services Inc., Marietta, OH) with free access to food and water.

### In Situ Hybridization

Unfixed dorsal skin samples (12-16 μm thick sections) were collected and embedded in OCT. In situ hybridization was performed using an ACD RNAScope kit (2.5 HD assay-Red) according to the manufacturer's protocol with the following modifications. For Fgf18 (495421) in situ, the slides were incubated for 40 minutes with Protease III and incubated for 45 minutes with AmpS. For Shh (314361), in situ was performed according to the manufacturer's protocol.

### Adrenalectomy

P19 C57BL/6J mice were anesthetized. Both adrenal glands were removed using curved forceps through 2 small incisions. Sex- and aged-matched controls underwent a sham operation using an identical procedure, except their adrenal glands were not removed. Adrenalectomized mice were given drinking water with 1% NaCl following surgery and were provided with corticosterone supplemented water from P21 onwards. Sham mice were provided with vehicle solution. Corticosterone water was prepared by dissolving 35 μg/ml corticosterone (Sigma 27840) in 0.66% (2-Hydroxypropyl)-β-cyclodextrin (Sigma 778966).

### Hormone Measurements

For corticosterone, norepinephrine, and epinephrine measurements following adrenalectomy and sham surgery, blood plasma was used. Following euthanasia, blood was collected from the heart, transferred to Microvette 300 Capillary Blood Collection Tubes (Fischer Scientific 22-043975), and centrifuged at 3000 g for 2 minutes. Plasma was transferred to new tubes and stored at −80° C. prior to hormone measurements. Hormone measurements were performed using the following ELISA kits, according to the manufacturers' protocols: Corticosterone ELISA kit (ARBOR ASSAYS, KO14-H1), Epinephrine ELISA kit (Abnova KA3837), and Norepinephrine ELISA kit (Abnova KA1891).

For norepinephrine measurements in the skin (following sympathectomy or cold exposure), a 4-mm punch biopsy was used to collect full thickness skin (approximately 25 mg). The skin was homogenized in 40 μl lysis buffer, and norepinephrine concentration was determined using a Norepinephrine ELISA kit (MYBioSource, MBS2600834) according to the manufacturer's protocol.

### AAV Generation and Administration

The following constructs were used: pAAV-CAG-tdTomato (Addgene plasmid #59462) was used to generate AAV-PHP.S-CAG-tdTomato virus (Addgene); and pAAV-mCherry-flex-DTA (Addgene plasmid #58536) was used to generate AAV2/PHP.S-mCherry-flex-DTA virus (BCH viral core). For FGF18 overexpression, the coding sequence of Fgf18 (with HA tag) was cloned into the pAAV backbone. The plasmid was further used to generate AAV8-CAG-FGF18-3XHA virus (Welgen).

All AAV viruses were injected intradermally. Viral stock was diluted to a concentration of 1E12 gc/ml with saline (0.9% NaCl). 50 μl of the diluted virus was injected once intradermally. Dorsal skin was collected 6 days following injection of AAV-PHP.S-CAG-tdTomato, 10 days following AAV8-CAG-FGF18-3XHA injection, and 18 days following AAV2/PHP.S-mCherry-flex-DTA. For APM ablation, AAV2/PHP.S-mCherry-flex-DTA was injected as described above into Myh11 -CreER mice.

### Doxycycline Administration

Timed-pregnant females were administrated with 300 μl of Doxycycline (Sigma D3447 10 mg/ml) by oral gavage and switched to a Doxycycline rodent diet (S3888) from E15.5.

### Topical Tamoxifen Treatment

A solution of 20 mg/ml Tamoxifen (Sigma T5648) in 100% ethanol was used for topical Tamoxifen treatment. The dorsal skin of Myh11-CreER; Rosa-lsl-YFP mice was shaved prior to treatment. Tamoxifen (100 μl) was applied topically once a day during first telogen at P20-P22. A solution of 10 mg/ml 4-Hydroxytamoxifen (Sigma H6278) in 100% ethanol was used for all 4-Hydroxytamoxifen topical treatments. The dorsal skin of TH-CreER; Rosa-lsl-DTA mice was shaved prior to treatment. 4-Hydroxytamoxifen (100 μl) was applied topically once a day at P20-P24. For APM ablation, AAV2/PHP.S-mCherry-flex-DTA was injected as described above into Myh11 -CreER mice. 4 days following injection, 4-Hydroxytamoxifen (200 μl) was applied topically once a day for 6 days. Control mice were treated with 100% ethanol.

### Colony Formation Assay (CFA)

Human hair follicles were isolated from normal scalp tissues. To isolate hair follicle stem cells (HFSCs), hair follicles were dissected, and subcutaneous fat and connective tissues were carefully removed with a scalpel. The lower hair bulb and upper epithelial layer were removed as previously described (Oshima et al., 2001; Rochat et al., 1994). For HFSC isolation, hair follicles were incubated in 1.25 U/mL dispase (Gibco) and 0.5 mg/mL collagenase I (Sigma-Aldrich) solution at 37° C. for 30 minutes, following which the mesenchymal sheath was carefully removed with forceps. To obtain a single cell HFSC suspension, tissue was digested with 0.05% trypsin/EDTA solution (Gibco) for 1 h at 37° C., and cells were filtered through a sterile 40-1 μm cell strainer (BD Biosciences). Single cell suspensions were then centrifuged at 1300 rpm for 10 minutes and plated on mitomycin C (Cayman) treated J2 feeders at a cell density of 5000 cells/well in a 12-well culture plate (Falcon) in E media supplemented with EGF and additives as described in (Mou et al., 2016; Nowak and Fuchs, 2009). After 48 hours, 0.1 μM procaterol (Sigma) was added (freshly prepared). Medium was changed every 2 days. On day 10, plates were fixed with 4% PFA and stained with Rhodamine B (Sigma). All experiments involving human samples were approved by Institutional Review Board of National Taiwan University and informed consent was obtained from patients undergoing routine scalp skin surgery. CFA quantification was done using Fiji.

### Sympathetic Nerve Ablation

Chemical ablation: 6-Hydroxydopamine hydrobromide (6-OHDA, Sigma 162957) solution was prepared freshly by dissolving 6-OHDA in 0.1% ascorbic acid (in 0.9% sterile NaCl) for intradermal injection. For intradermal injection, 0.6 mg of 6-OHDA was dissolved in 100 μl 0.1% ascorbic acid, and mice were injected at P18 or P19. Control animals were injected with vehicle (100 μl of 0.1% ascorbic acid). For norepinephrine measurements in the skin, following sympathetic nerve ablation, 7-week-old mice were used. 6-Hydroxydopamine hydrobromide (6-OHDA, Sigma 162957) solution was prepared freshly by dissolving 6-OHDA in 0.2% ascorbic acid (in 0.9% sterile NaCl). Mice were injected intraperitoneally for two consecutive days with the following doses: 250 mg/kg body weight and 100 mg/kg body weight. Skin was analyzed one week after ablation.

### EdU Administration

Two doses of EdU were administered by intraperitoneal injection before harvesting. The first injection was done 8 hours before harvesting, and the second one was done 4 hours before harvesting. 25 μg EdU/g body weight was injected each time (dissolved in 0.9% NaCl).

### Histology and Immunohistochemistry

Dorsal skin samples were fixed for 15 minutes using 4% paraformaldehyde (PFA) at room temperature, washed with PBS, immersed in 30% sucrose overnight at 4° C., and embedded in OCT (Sakura Finetek). 50-μm sections were used for all staining unless otherwise noted. For all 50-μm thick immunofluorescent staining, slides were blocked (5% Donkey serum, 1% BSA, 2% Cold water fish gelatin, and 0.3% Triton in PBS) for 1-4 hours at room temperature, incubated with primary antibody overnight at 4° C., then incubated with secondary antibody for 2-4 hours at room temperature or overnight at 4° C. For FIGS. 4A-4C, FIGS. 11A-11B, and FIG. 5D, 100-μm thick sections were used. Slides were blocked (5% Donkey serum, 1% BSA, 2% Cold water fish gelatin, and 0.3% Triton in PBS) for 1-4 hours at room temperature, incubated with primary antibody for 48 hours at 4° C., then incubated with secondary antibody for 48 hours at 4° C. For FIGS. 12B-12C, dorsal skin was fixed in 4% PFA at 4° C. overnight. Samples were washed with PBS and embedded in OCT for 100-μm thick sectioning. Sections were blocked (PBS, 5% BSA, 1% Tween20) for 12 hours at 4° C., incubated with primary antibodies for 2 days at 4° C., then incubated with secondary antibodies for 2 days at 4° C. The following antibodies and dilutions were used: CD34 (rat, eBioscience 14-0341-85, 1:100); phospho-histone H3 (rabbit, Cell Signaling Technology 3377S, 1:250); cleaved Caspase 3 (rabbit, Cell Signaling Technology 9664S, 1:100-1:300); PCAD (goat, R&D AF761, 1:400); Tyrosine hydroxylase (rabbit, Millipore AB152, 1:1000; sheep, Millipore AB1542, 1:150-1:300 or chicken, Millipore AB9720, 1:50); GFP (rabbit, Abcam ab290, 1:5000 or chicken, Ayes labs GFP 1010, 1:200); Integrin alpha 8 (goat, R&D AF4076, 1:200); TUJj1 (rabbit, Sigma T2200, 1:1000); Keratin 8 (rat, Developmental studies hybridoma bank TROMA-I, 1:200); Synaptotagmin 1/2 (rabbit, Synaptic Systems 105003, 1:500); Synaptophysin (rabbit, Thermo Fisher Scientific MA514523, 1:100); Smooth Muscle Actin (rabbit, Abcam ab5694, 1:800 or mouse, anti-SMA-Cy3, Sigma C6198, 1:300); CD31 (rat, Abcam ab56299, 1:100 or rat, BD Biosciences 550274, 1:50); Nephronectin/NPNT (goat, R&D System AF4298, 1:200); HA antibody (rabbit, Cell Signaling 3724s, 1:200); Vesicular monoamine transporter 2 (rabbit, Synaptic Systems 138313, 1:500); SOX9 (rabbit, EMD Millipore AB5535, 1:500); Keratin 82 (guinea pig, ORIGENE BP5091, 1:200); GATA3 (rat, Thermo Fisher Scientific 14-9966-80, 1:100); Keratin 6 (rabbit, BioLegend 905702/Covance PRB-169P, 1:1000); CD140a (goat, R&D Systems AF1062-SP, 1:200); tdTomato (rat, Kerafast EST203, 1:500); Beta galactosidase (rabbit, MP Bio 559761, 1:2500); and Keratin 14 (rabbit, BioLegened PRB-155P, 1:800). For c-FOS staining, sympathetic ganglia chain was freshly embedded in OCT. 40-μm thick sections were fixed in 2% PFA for 5 minutes, washed in 0.3% Triton in PBS, and incubated with 0.1 M glycine for 5 minutes. Slides were then washed, blocked (5% Donkey serum; 1% BSA, 2% Cold water fish gelatin, and 0.3% Triton in PBS) for 1-4 hours at room temperature, incubated with primary c-FOS antibody (rabbit, Abcam ab190289, 1:2000) overnight at 4° C., and then incubated with secondary antibody for 2-4 hours at room temperature or overnight at 4° C. For nuclear counter staining, samples were incubated in 1 μg/ml DAPI (Sigma) for 2-4 hours at room temperature or overnight at 4° C. EdU was developed for 1 hour, using the Click-It reaction according to the manufacturer's instructions (Thermo Fisher Scientific). Hematoxylin and Eosin (H&E) staining and Masson's staining were performed according to standard protocols with the following timing modifications for early post-natal samples: For Masson's trichrome staining, 20-μm sections were incubated in Weigert's iron hematoxylin (Solution A+B) for 2 minutes, in scarlet acid solution for 3 minutes, and in aniline blue solution for 1.5 minutes. For H&E, 20-50-μm thick sections were incubated for 2 minutes in hematoxylin and 3 minutes in eosin solutions.

### FACS

FACS was used to isolate first telogen HFSCs in control and K15-CrePGR; Adrb2 fl/fl male mice. Mouse back skin was dissected, and the fat layer was scraped using a surgical scalpel. The skin was incubated in trypsin-EDTA at 37° C. for 35-45 minutes on an orbital shaker. Single cell suspension was obtained by scraping the epidermal side and filtering through 70-μm and 40-μm filters. Single cell suspensions were then centrifuged for 8 minutes at 350g at 4° C., re-suspended in 5% FBS and stained for 30-45 minutes. The following antibodies were used: CD49f-PE-Integrin alpha 6 (eBioscience 12-0495-82, 1:500); CD34-eF660 (eBioscience 50-0341-82, 1:100); Ly-6A/E (Sca-1)-PerCp-Cy5.5 (eBioscience 45-5981-82, 1:1000); and CD45-eF450 (eBioscience 48-0451-82, 1:250). DAPI (Sigma) was used to exclude dead cells. HFSCs were isolated as CD45 negative, Integrin alpha 6+, CD34+, Sca-1 negative cells. Cell isolation was performed with BD-Aria sorters.

RNA Isolation

First telogen HFSCs from control and K15-CrePGR; Adrb2 fl/fl male mice were FACS sorted and collected into TRIzol® LS Reagent (Invitrogen). RNA was isolated with an RNeasy Micro Kit (Qiagen), using a QlAcube according to the manufacturer's instructions. RNA concentration and RNA integrity were determined by Bioanalyzer (Agilent, Santa Clara, CA) using the RNA 6000 Nano chip. High quality RNA samples with RNA Integrity Number≥8 were used as input for RT-PCR and RNA-sequencing. For Shh, Dhh, and Ihh quantitative real time PCR, newborn pups were used. The mouse back skin was dissected. The skin was incubated with 0.25% Collagenase (Sigma c2674) in Hank's Balanced Salt Solution (HBSS) at 37° C. for 20-35 minutes on an orbital shaker. The dermal side was scraped, and cells were collected and incubated for 10 minutes in trypsin-EDTA at 37° C. to generate a single cell suspension. The remaining tissue was incubated in trypsin-EDTA at 37° C. and scraped again. All cells were collected together and filtered through 70-μm and 40-μm filters. Single cell suspensions were then centrifuged for 8 minutes at 350g at 4° C. and re-suspended in TRIzol® LS Reagent (Invitrogen). RNA isolation was performed using ZYMO RESEARCH Direct-Zol RNA Micro-Prep kit (zr2060) according to the manufacturer's protocol.

### Quantitative Real-Time PCR

The cDNA libraries were synthesized using Superscript IV VILO master mix with ezDNase (Thermo Fisher). Quantitative real time PCR was performed using power SYBR green (Thermo Fisher). Ct values were normalized to beta-actin.

| Primer: | Sequence: |
|---|---|
| Adrb2-set1-forward | TGGGGCCAGTCACATCCTTAT (SEQ ID NO: 1) |
| Adrb2-set1-reverse | TGACGCACAACACATCAATGG (SEQ ID NO: 2) |
| Adrb2-set2-forward | TACACAGGGGAGCCAAACAC (SEQ ID NO: 3) |
| Adrb2-set2-reverse | TCACAAAGCCTTCCATGCCT (SEQ ID NO: 4) |
| B-actin forward | CCTGTATGCCTCTGGTCGTA (SEQ ID NO: 5) |
| B-actin reverse | CCATCTCCTGCTCGAAGTCT (SEQ ID NO: 6) |
| Foxp1-forward | GTCTTGTGGCGTTCTGCA (SEQ ID NO: 7) |
| Foxp1-reverse | GCTGGACCCGTTCTGGAT (SEQ ID NO: 8) |
| Fgf18-forward | CCCAGGACTTGAATGTGCTT (SEQ ID NO: 9) |
| Fgf18-reverse | ACTGCTGTGCTTCCAGGTTC (SEQ ID NO: 10) |
| Shh-forward | GGGACCGCAGCAAGTACGGC (SEQ ID NO: 11) |
| Shh-reverse | CGGATTTGGCCGCCACGGAG (SEQ ID NO: 12) |
| Dhh-forward | GGTAACAAGGGGGTCGGAG (SEQ ID NO: 13) |
| Dhh-reverse | TTGCAACGCTCTGTCATCAG (SEQ ID NO: 14) |
| Ihh-forward | CTCTTGCCTACAAGCAGTTCA (SEQ ID NO: 15) |
| Ihh-reverse | CCGTGTTCTCCTCGTCCTT (SEQ ID NO: 16) |

### RNA-Sequencing and Analysis

RNA-sequencing libraries were prepared using 1 ng of total RNA as input. SMART-Seq v4 Ultra Low Input RNA Kit for Sequencing (Takara) was used for cDNA synthesis, with a 10 cycle PCR enrichment. Sequencing libraries were then made using Illumina's Nextera XT Library Prep kit. A modified quarter-volume reaction protocol was used for both kits. The indexed libraries were sequenced over two flow cells on a NextSeq High-Output platform using the unpaired, 75-bp read-length sequencing protocol to obtain a total of at least 10 million reads per sample. Sequencing reads were aligned to the mouse genome (mm10) using Salmon (Patro et al., 2017). Differential expression analysis was performed using DESeq2 (Love et al., 2014). Statistical significance was given to genes using adjusted P value of 0.1 according to Benjamini-Hochberg adjustment with FDR=0.1 and absolute fold change bigger than 2. Pathway analyses were performed using Ingenuity Pathway Analysis (IPA-QIAGEN) and Gene Ontology (GO) for the statistically significant genes. Heatmaps were generated using TPM values of all sequenced genes. The accession number for RNA-sequencing raw and analyzed data in GEO is GSE130240.

### Hair Cycle Staging

H&E stained sections were used for analysis. For quantification, anagen II and anagen III were considered as early anagen, anagen IV was mid anagen, and anagen V and anagen VI were full anagen. Hair cycle stages were determined using previously described criteria (Muller-Rover et al., 2001). Ten to twenty hair follicles were individually assessed and staged in each animal, and at least 4 different animals were used per condition. Hair cycle staging in control and sympathectomized (6-OHDA injected and TH-CreER; Rosa-lsl-attenuated DTA mice) mice was performed on sections from the treated (6-OHDA injected or treated with 4-Hydroxytamoxifen) area at P30-P34. For comparison, 6-OHDA and vehicle were always injected in the same position of the back skin. For hair cycle staging of Adrb2-cKO mice, a biopsy was taken from similar anatomical locations in control and Adrb2-cKO (only males were used for the analysis). Hair cycle staging in control and AAV8-CAG-FGF18-3XHA injected mice was performed on sections from the injected site. Hair cycle staging in sham and ADX+CORT mice was perform on dorsal skin sections. Only animals with comparable plasma corticosterone levels were used (as measured by ELISA). The effects of cold exposure and adrenergic agonist treatment (isoproterenol and procaterol) on anagen entry were quantified by monitoring the change of hair regrowth as previously described (Fan et al., 2018; Sheen et al., 2015). The percent of dorsal skin in anagen was quantified using Fiji. For all analyses, sex- and aged-matched mice were used.

Electron mMicroscopy

P21 back skin was dissected and fixed using 4% PFA, 2.5% glutaraldehyde in 0.1 M sodium cacodylate buffer. Samples were submitted for further processing (staining, embedding, sectioning, and imaging) to Renovo Neural Inc. (Cleveland) for serial section TEM (80 nM per slice, 8-10 nM per pixel). Three independent hair follicles were analyzed. For 3D analysis, EM images were manually segmented and rendered using VAST lite. To visualize neurotransmitter positive vesicles, skin samples were fixed in 2.5% glutaraldehyde, 1.25% paraformaldehyde, and 0.03% picric acid in 0.1 M sodium cacodylate buffer (pH 7.4), washed in 0.1 M cacodylate buffer, and post-fixed with 1% osmium tetroxide (0s04) in 1.5% potassium ferrocyanide (KFeCN6) for 1 hour, washed twice in water, washed once in Maleate buffer (MB), incubated in 1% uranyl acetate in MB for 1 hour followed by 2 washes in water, and subsequently dehydrated in grades of alcohol (10 minutes each; 50%, 70%, 90%, 2×10 minutes 100%). The samples were then put in propyleneoxide for 1 hour and infiltrated overnight in a 1:1 mixture of propyleneoxide and Spurr's low viscosity resin (Electron Microscopy Sciences, Hatfield, PA). The following day the samples were embedded in Spurr's resin and polymerized at 60° C. for 48 hours. Ultrathin sections (about 80 nm) were cut on a Reichert Ultracut-S microtome, picked up on to copper grids stained with lead citrate, and examined in a JEOL 1200EX. Transmission electron microscope images were recorded with an AMT 2k CCD camera.

### RU486 Treatment

For topical treatment, 4% Mifepristone (TCI America, M1732) in ethanol was used to induce K15-CrePGR. The dorsal skin of the mice was shaved prior to treatment. RU486 was applied topically 10-14 times once a day to both control and K15-CrePGR; Adrb2 fl/fl mice.

### Adrenergic Agonist Topical Application

Procaterol (10 mg/kg body weight, Sigma P9180) or isoproterenol (10 mg/kg body weight, Sigma 15627) were dissolved in hand cream (Neutrogena Norwegian Formula Concentrated Hand Cream) at 10 mg drug/1 g cream concentration. The dorsal skin was shaved prior to treatment. The isoproterenol/procaterol-cream was applied topically once a day for 10 days. Cream without agonists was applied to control mice.

### Diphtheria Toxin Administration

Diphtheria toxin (Sigma-Aldrich) was dissolved in 0.9% NaCl (0.1 mg/ml). For APM ablation, 8-week-old SMA-YFP-DTR transgenic mice were intradermally injected with 250 ng/kg diphtheria toxin.

### Imaging and Image Analysis

All images were acquired using a Zeiss LSM 880, LSM 700 confocal microscope, or Keyence microscope using x10, x20 or x63 magnification lenses. Images are presented as either a Maximum Intensity Projection image or a single Z stack. For image analysis, Imaris software (Oxford Instruments) and Fiji (Schindelin et al., 2012) were used. The following analyses were performed using Imaris software:

(1) Co-localization between YFP and ITGA8 was quantified using the Imaris colocalization module. For each analyzed APM, a region of interest covering the entire APM was defined and used for all measurements. Seven to twelve muscles were analyzed in each animal, and 3 animals were used for analysis at all time points. Outlining of the APM was performed according to ITGA8 staining for both channels. For YFP and ITGA8 co-localization, the value of “% of material above threshold colocalized” was used.

(2) Quantification of SMA+blood vessels. First a CD31+volume was automatically created. Using the “distance transformation” and “mask” functions, a second SMA+volume up to 2 μm from CD31+cells was created. To quantify the percent of endothelial cells volume covered by SMA+, this volume (SMA+ volume, 2 μm from CD31+ staining) was divided by the total CD31+volume and presented as a percentage. Three to seven 20× confocal images were quantified per animal. Three control and 3 SMA-YFP-DTR animals were used. For Fgf18 in situ quantification, bright field images were used. Fgf18+ spots in the outer bulge (HFSC) area were manually counted using Fiji. Seven to eleven hair follicles were quantified per animal, and 3 animals were used for each condition (3 control and 3 K15-CrePGR; Adrb2 fl/fl). For c-FOS+ quantification in sympathetic ganglia, TH and c-FOS stained sections were used. TH staining was used to identify the sympathetic ganglia. The total number of cells (TH+) as well as c-FOS+ positive cells were manually quantified using Fiji. Three to five sympathetic ganglia were quantified per animal, and 2 animals per condition (cold and control) were used. For quantification of hair follicles with APM during development, dorsal skin was used. Using Fiji, the number of hair follicles and APMs was manually counted. Results are presented as (number of APM/number of hair follicle)×100. For innervation frequency analysis: First telogen maximum projection 20× images were used. For each hair follicle, HFSCs were divided into four compartments: upper bulge, mid bulge, lower bulge, and hair germ. For every quantified hair follicle, the innervation pattern was analyzed and each HFSC compartment was scored “1” if innervation was present or “0” if there was no innervation. Ten to thirty hair follicles were individually assessed in each animal, and 2-3 different animals were used for quantification.

### Analysis of Published Datasets

For RNA-seq data of adrenergic receptors, the following datasets were used: Ge Y et al., 2017, GEO accession GSE89928 and Lay et al., 2016 PNAS, GEO accession GSE77256. For ChIP-seq of adrenergic receptors the following dataset was used: Lien W H, 2011 Cell Stem Cell, GEO accession GSE31239.

### Quantification and Statistical Analysis

Statistical analyses were performed with Prism using unpaired two-tailed Student's t-test. Statistical significance is denoted by asterisks (P<0.05 [*], P<0.01 [**], and P<0.0001 [***]. The data are presented as mean±SEM. All statistical details (including the value of n and what it represents) can be found in figures and figure legends.

### REFERENCE

Abraira, V. E., Kuehn, E. D., Chirila, A. M., Springel, M. W., Toliver, A. A., Zimmerman, A. L., Orefice, L. L., Boyle, K. A., Bai, L., Song, B. J., et al. (2017). The Cellular and Synaptic Architecture of the Mechanosensory Dorsal Horn. Cell 168, 295-310 e219.

Andl, T., Reddy, S. T., Gaddapara, T., and Millar, S. E. (2002). WNT signals are required for the initiation of hair follicle development. Dev Cell 2, 643-653.

Asada-Kubota, M. (1995). Inhibition of hair growth by subcutaneous injection of a sympathetic neurotoxin, 6-hydroxydopamine in neonatal mice. Anat Embryol (Berl) 191, 407-414.

Bai, C. B., Auerbach, W., Lee, J. S., Stephen, D., and Joyner, A. L. (2002). Gli2, but not Glil, is required for initial Shh signaling and ectopic activation of the Shh pathway. Development 129, 4753-4761.

Belteki, G., Haigh, J., Kabacs, N., Haigh, K., Sison, K., Costantini, F., Whitsett, J., Quaggin, S. E., and Nagy, A. (2005). Conditional and inducible transgene expression in mice through the combinatorial use of Cre-mediated recombination and tetracycline induction. Nucleic Acids Res 33, e51.

Bitgood, M. J., Shen, L., and McMahon, A. P. (1996). Sertoli cell signaling by Desert hedgehog regulates the male germline. Curr Biol 6, 298-304.

Borden, P., Houtz, J., Leach, S. D., and Kuruvilla, R. (2013). Sympathetic innervation during development is necessary for pancreatic islet architecture and functional maturation. Cell Rep 4, 287-301.

Botchkarev, V. A., Peters, E. M., Botchkareva, N. V., Maurer, M., and Paus, R. (1999). Hair cycle-dependent changes in adrenergic skin innervation, and hair growth modulation by adrenergic drugs. J Invest Dermatol 113, 878-887.

Brownell, I., Guevara, E., Bai, C. B., Loomis, C. A., and Joyner, A. L. (2011). Nerve-derived sonic hedgehog defines a niche for hair follicle stem cells capable of becoming epidermal stem cells. Cell Stem Cell 8, 552-565.

Burnstock, G. (2008). Non-synaptic transmission at autonomic neuroeffector junctions. Neurochem Int 52, 14-25.

Chen, C. L., Huang, W. Y., Wang, E. H. C., Tai, K. Y., and Lin, S. J. (2020). Functional complexity of hair follicle stem cell niche and therapeutic targeting of niche dysfunction for hair regeneration. J Biomed Sci 27, 43.

Chiang, C., Swan, R. Z., Grachtchouk, M., Bolinger, M., Litingtung, Y., Robertson, E. K., Cooper, M. K., Gaffield, W., Westphal, H., Beachy, P. A., et al. (1999). Essential role for Sonic hedgehog during hair follicle morphogenesis. Dev Biol 205, 1-9.

Crowe, R., Mitsou, J., McGrouther, D. A., and Burnstock, G. (1993). An increase in the growth of hair associated with hyperinnervation of the underlying vessels in rabbit skin. Neurosci Lett 161, 105-108.

Darwin, C. R. (1872). The expression of the emotions in man and animals (London: John Murray).

Dassule, H. R., Lewis, P., Bei, M., Maas, R., and McMahon, A. P. (2000). Sonic hedgehog regulates growth and morphogenesis of the tooth. Development 127, 4775-4785.

Driskell, R. R., Lichtenberger, B. M., Hoste, E., Kretzschmar, K., Simons, B. D., Charalambous, M., Ferron, S. R., Herault, Y., Pavlovic, G., Ferguson-Smith, A. C., et al. (2013). Distinct fibroblast lineages determine dermal architecture in skin development and repair. Nature 504, 277-281.

Fan, S. M., Chang, Y. T., Chen, C. L., Wang, W. H., Pan, M. K., Chen, W. P., Huang, W. Y., Xu, Z., Huang, H. E., Chen, T., et al. (2018). External light activates hair follicle stem cells through eyes via an ipRGC-SCN-sympathetic neural pathway. Proc Natl Acad Sci U S A 115, E6880-E6889.

Fujiwara, H., Ferreira, M., Donati, G., Marciano, D. K., Linton, J. M., Sato, Y., Hartner, A., Sekiguchi, K., Reichardt, L. F., and Watt, F. M. (2011). The basement membrane of hair follicle stem cells is a muscle cell niche. Cell 144, 577-589.

Furlan, A., La Manno, G., Lubke, M., Haring, M., Abdo, H., Hochgerner, H., Kupari, J., Usoskin, D., Airaksinen, M. S., Oliver, G., et al. (2016). Visceral motor neuron diversity delineates a cellular basis for nipple- and pilo-erection muscle control. Nat Neurosci 19, 1331-1340.

Ge, Y., Gomez, N. C., Adam, R. C., Nikolova, M., Yang, H., Verma, A., Lu, C. P., Polak, L., Yuan, S., Elemento, 0., et al. (2017). Stem Cell Lineage Infidelity Drives Wound Repair and Cancer. Cell 169, 636-650 e614.

Goldstein, J. M., Tabebordbar, M., Zhu, K., Wang, L. D., Messemer, K. A., Peacker, B., Ashrafi Kakhki, S., Gonzalez-Celeiro, M., Shwartz, Y., Cheng, J. K. W., et al. (2019). In Situ Modification of Tissue Stem and Progenitor Cell Genomes. Cell Rep 27, 1254-1264 e1257.

Greco, V., Chen, T., Rendl, M., Schober, M., Pasolli, H. A., Stokes, N., Dela Cruz-Racelis, J., and Fuchs, E. (2009). A two-step mechanism for stem cell activation during hair regeneration. Cell Stem Cell 4,155-169.

Heidt, T., Sager, H. B., Courties, G., Dutta, P., Iwamoto, Y., Zaltsman, A., von Zur Muhlen, C., Bode, C., Fricchione, G. L., Denninger, J., et al. (2014). Chronic variable stress activates hematopoietic stem cells. Nat Med 20, 754-758.

Heitman, N., Sennett, R., Mok, K. W., Saxena, N., Srivastava, D., Martino, P., Grisanti, L., Wang, Z., Ma'ayan, A., Rompolas, P., et al. (2020). Dermal sheath contraction powers stem cell niche relocation during hair cycle regression. Science 367, 161-166.

Hinoi, E., Gao, N., Jung, D. Y., Yadav, V., Yoshizawa, T., Myers, M. G., Jr., Chua, S. C., Jr., Kim, J. K., Kaestner, K. H., and Karsenty, G. (2008). The sympathetic tone mediates leptin's inhibition of insulin secretion by modulating osteocalcin bioactivity. J Cell Biol 183, 1235-1242.

Hsu, Y. C., Li, L., and Fuchs, E. (2014a). Emerging interactions between skin stem cells and their niches. Nat Med 20, 847-856.

Hsu, Y. C., Li, L., and Fuchs, E. (2014b). Transit-amplifying cells orchestrate stem cell activity and tissue regeneration. Cell 157, 935-949.

Hsu, Y. C., Pasolli, H. A., and Fuchs, E. (2011). Dynamics between stem cells, niche, and progeny in the hair follicle. Cell 144, 92-105.

Kamiya, A., Hayama, Y., Kato, S., Shimomura, A., Shimomura, T., Irie, K., Kaneko, R., Yanagawa, Y., Kobayashi, K., and Ochiya, T. (2019). Genetic manipulation of autonomic nerve fiber innervation and activity and its effect on breast cancer progression. Nat Neurosci 22, 1289-1305.

Karemaker, J. M. (2017). An introduction into autonomic nervous function. Physiol Meas 38, R89-R118.

Katayama, Y., Battista, M., Kao, W. M., Hidalgo, A., Peired, A. J., Thomas, S. A., and Frenette, P. S. (2006). Signals from the sympathetic nervous system regulate hematopoietic stem cell egress from bone marrow. Cell 124, 407-421.

Kimura-Ueki, M., Oda, Y., Oki, J., Komi-Kuramochi, A., Honda, E., Asada, M., Suzuki, M., and Imamura, T. (2012). Hair cycle resting phase is regulated by cyclic epithelial FGF18 signaling. J Invest Dermatol 132, 1338-1345.

Kobayasi, S., Okuyama, F., and Takagi, K. (1958). Experimental studies on the hemitrichosis and the nervous influences on the hair growth. Acta Neuroveg (Wien) 18, 169-190.

Kong, Y., Liu, Y., Pan, L., Cheng, B., and Liu, H. (2015). Norepinephrine Regulates Keratinocyte Proliferation to Promote the Growth of Hair Follicles. Cells Tissues Organs 201, 423-435.

Kostrzewa, R. M., and Jacobowitz, D. M. (1974). Pharmacological actions of 6-hydroxydopamine. Pharmacol Rev 26, 199-288.

Lay, K., Kume, T., and Fuchs, E. (2016). FOXC1 maintains the hair follicle stem cell niche and governs stem cell quiescence to preserve long-term tissue-regenerating potential. Proc Natl Acad Sci U S A 113, E1506-1515.

Leishman, E., Howard, J. M., Garcia, G. E., Miao, Q., Ku, A. T., Dekker, J. D., Tucker, H., and Nguyen, H. (2013). Foxpl maintains hair follicle stem cell quiescence through regulation of Fgf18. Development 140, 3809-3818.

Lewis, P. M., Dunn, M. P., McMahon, J. A., Logan, M., Martin, J. F., St-Jacques, B., and McMahon, A. P. (2001). Cholesterol modification of sonic hedgehog is required for long-range signaling activity and effective modulation of signaling by Ptcl. Cell 105, 599-612.

Li, L., and Ginty, D. D. (2014). The structure and organization of lanceolate mechanosensory complexes at mouse hair follicles. Elife 3, e01901.

Lien, W. H., Guo, X., Polak, L., Lawton, L. N., Young, R. A., Zheng, D., and Fuchs, E. (2011). Genome-wide maps of histone modifications unwind in vivo chromatin states of the hair follicle lineage. Cell Stem Cell 9, 219-232.

Llorens-Bobadilla, E., Zhao, S., Baser, A., Saiz-Castro, G., Zwadlo, K., and Martin-Villalba, A. (2015). Single-Cell Transcriptomics Reveals a Population of Dormant Neural Stem Cells that Become Activated upon Brain Injury. Cell Stem Cell 17, 329-340.

Long, F., Zhang, X. M., Karp, S., Yang, Y., and McMahon, A. P. (2001). Genetic manipulation of hedgehog signaling in the endochondral skeleton reveals a direct role in the regulation of chondrocyte proliferation. Development 128, 5099-5108.

Love, M. I., Huber, W., and Anders, S. (2014). Moderated estimation of fold change and dispersion for RNA-seq data with DESeq2. Genome Biol 15, 550.

Lucas, D., Scheiermann, C., Chow, A., Kunisaki, Y., Bruns, I., Barrick, C., Tessarollo, L., and Frenette, P. S. (2013). Chemotherapy-induced bone marrow nerve injury impairs hematopoietic regeneration. Nat Med 19, 695-703.

Maryanovich, M., Zahalka, A. H., Pierce, H., Pinho, S., Nakahara, F., Asada, N., Wei, Q., Wang, X., Ciero, P., Xu, J., et al. (2018). Adrenergic nerve degeneration in bone marrow drives aging of the hematopoietic stem cell niche. Nat Med 24, 782-791.

Morris, R. J., Liu, Y., Marles, L., Yang, Z., Trempus, C., Li, S., Lin, J. S., Sawicki, J. A., and Cotsarelis, G. (2004). Capturing and profiling adult hair follicle stem cells. Nat Biotechnol 22, 411-417.

Mou, H., Vinarsky, V., Tata, P. R., Brazauskas, K., Choi, S. H., Crooke, A. K., Zhang, B., Solomon, G. M., Turner, B., Bihler, H., et al. (2016). Dual SMAD Signaling Inhibition Enables Long-Term Expansion of Diverse Epithelial Basal Cells. Cell Stem Cell 19, 217-231.

Muller-Rover, S., Handjiski, B., van der Veen, C., Eichmuller, S., Foitzik, K., McKay, I. A., Stenn, K. S., and Paus, R. (2001). A comprehensive guide for the accurate classification of murine hair follicles in distinct hair cycle stages. J Invest Dermatol 117, 3-15.

Nowak, J. A., and Fuchs, E. (2009). Isolation and culture of epithelial stem cells. Methods Mol Biol 482, 215-232.

Oshima, H., Rochat, A., Kedzia, C., Kobayashi, K., and Barrandon, Y. (2001). Morphogenesis and renewal of hair follicles from adult multipotent stem cells. Cell 104, 233-245.

Patro, R., Duggal, G., Love, M. I., Irizarry, R. A., and Kingsford, C. (2017). Salmon provides fast and bias-aware quantification of transcript expression. Nat Methods 14, 417-419.

Perdigoto, C. N., Dauber, K. L., Bar, C., Tsai, P. C., Valdes, V. J., Cohen, I., Santoriello, F. J., Zhao, D., Zheng, D., Hsu, Y. C., et al. (2016). Polycomb-Mediated Repression and Sonic Hedgehog Signaling Interact to Regulate Merkel Cell Specification during Skin Development. PLoS Genet 12, e1006151.

Peters, E. M., Maurer, M., Botchkarev, V. A., Gordon, D. S., and Paus, R. (1999). Hair growth-modulation by adrenergic drugs. Exp Dermatol 8, 274-281.

Peterson, S. C., Eberl, M., Vagnozzi, A. N., Belkadi, A., Veniaminova, N. A., Verhaegen, M. E., Bichakjian, C. K., Ward, N. L., Dlugosz, A. A., and Wong, S. Y. (2015). Basal cell carcinoma preferentially arises from stem cells within hair follicle and mechanosensory niches. Cell Stem Cell 16, 400-412.

Pruitt, S. C., Freeland, A., Rusiniak, M. E., Kunnev, D., and Cady, G. K. (2013). Cdkn1b overexpression in adult mice alters the balance between genome and tissue ageing. Nat Commun 4, 2626.

Rezza, A., Wang, Z., Sennett, R., Qiao, W., Wang, D., Heitman, N., Mok, K. W., Clavel, C., Yi, R., Zandstra, P., et al. (2016). Signaling Networks among Stem Cell Precursors, Transit-Amplifying Progenitors, and their Niche in Developing Hair Follicles. Cell Rep 14, 3001-3018.

Rivera-Gonzalez, G. C., Shook, B. A., Andrae, J., Holtrup, B., Bollag, K., Betsholtz, C., Rodeheffer, M. S., and Horsley, V. (2016). Skin Adipocyte Stem Cell Self-Renewal Is Regulated by a PDGFA/AKT-Signaling Axis. Cell Stem Cell 19, 738-751.

Rochat, A., Kobayashi, K., and Barrandon, Y. (1994). Location of stem cells of human hair follicles by clonal analysis. Cell 76, 1063-1073.

Rodgers, J. T., King, K. Y., Brett, J. 0., Cromie, M. J., Charville, G. W., Maguire, K. K., Brunson, C., Mastey, N., Liu, L., Tsai, C. R., et al. (2014). mTORC1 controls the adaptive transition of quiescent stem cells from GO to G(Alert). Nature 510, 393-396.

Roesch, K., Jadhav, A. P., Trimarchi, J. M., Stadler, M. B., Roska, B., Sun, B. B., and Cepko, C. L. (2008). The transcriptome of retinal Muller glial cells. J Comp Neurol 509, 225-238.

Rompolas, P., Mesa, K. R., and Greco, V. (2013). Spatial organization within a niche as a determinant of stem-cell fate. Nature 502, 513-518.

Rompolas, P., Mesa, K. R., Kawaguchi, K., Park, S., Gonzalez, D., Brown, S., Boucher, J., Klein, A. M., and Greco, V. (2016). Spatiotemporal coordination of stem cell commitment during epidermal homeostasis. Science 352, 1471-1474.

Sada, A., Jacob, F., Leung, E., Wang, S., White, B. S., Shalloway, D., and Tumbar, T. (2016). Defining the cellular lineage hierarchy in the interfollicular epidermis of adult skin. Nat Cell Biol 18, 619-631.

Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682.

Schmidt-Ullrich, R., and Paus, R. (2005). Molecular principles of hair follicle induction and morphogenesis. Bioessays 27, 247-261.

Sennett, R., Wang, Z., Rezza, A., Grisanti, L., Roitershtein, N., Sicchio, C., Mok, K. W., Heitman, N. J., Clavel, C., Ma'ayan, A., et al. (2015). An Integrated Transcriptome Atlas of Embryonic Hair Follicle Progenitors, Their Niche, and the Developing Skin. Dev Cell 34, 577-591.

Sheen, Y. S., Fan, S. M., Chan, C. C., Wu, Y. F., Jee, S. H., and Lin, S. J. (2015). Visible red light enhances physiological anagen entry in vivo and has direct and indirect stimulative effects in vitro. Lasers Surg Med 47, 50-59.

Sheu, S. H., Tapia, J. C., Tsuriel, S., and Lichtman, J. W. (2017). Similar synapse elimination motifs at successive relays in the same efferent pathway during development in mice. Elife 6, e23193.

Srinivas, S., Watanabe, T., Lin, C. S., William, C. M., Tanabe, Y., Jessell, T. M., and Costantini, F. (2001). Cre reporter strains produced by targeted insertion of EYFP and ECFP into the ROSA26 locus. BMC Dev Biol 1, 4.

St-Jacques, B., Dassule, H. R., Karavanova, I., Botchkarev, V. A., Li, J., Danielian, P. S., McMahon, J. A., Lewis, P. M., Paus, R., and McMahon, A. P. (1998). Sonic hedgehog signaling is essential for hair development. Curr Biol 8, 1058-1068.

Suo, D., Park, J., Young, S., Makita, T., and Deppmann, C. D. (2015). Coronin-1 and calcium signaling governs sympathetic final target innervation. J Neurosci 35, 3893-3902.

Swanson, L. W., and Lichtman, J. W. (2016). From Cajal to Connectome and Beyond. Annu Rev Neurosci 39, 197-216.

Torkamani, N., Rufaut, N. W., Jones, L., and Sinclair, R. (2014). Destruction of the arrector pili muscle and fat infiltration in androgenic alopecia. Br J Dermatol 170, 1291-1298.

van Velthoven, C. T. J., de Morree, A., Egner, I. M., Brett, J. O., and Rando, T. A. (2017). Transcriptional Profiling of Quiescent Muscle Stem Cells In Vivo. Cell Rep 21, 1994-2004.

Venkataramani, V., Tanev, D. I., Strahle, C., Studier-Fischer, A., Fankhauser, L., Kessler, T., Korber, C., Kardorff, M., Ratliff, M., Xie, R., et al. (2019). Glutamatergic synaptic input to glioma cells drives brain tumour progression. Nature 573, 532-538.

Venkatesh, H. S., Morishita, W., Geraghty, A. C., Silverbush, D., Gillespie, S. M., Arzt, M., Tam, L. T., Espenel, C., Ponnuswami, A., Ni, L., et al. (2019). Electrical and synaptic integration of glioma into neural circuits. Nature 573, 539-545.

Voehringer, D., Liang, H. E., and Locksley, R. M. (2008). Homeostasis and effector function of lymphopenia-induced “memory-like” T cells in constitutively T cell-depleted mice. J Immunol 180, 4742-4753.

Wang, L., Siegenthaler, J. A., Dowell, R. D., and Yi, R. (2016). Foxcl reinforces quiescence in self-renewing hair follicle stem cells. Science 351, 613-617.

Wirth, A., Benyo, Z., Lukasova, M., Leutgeb, B., Wettschureck, N., Gorbey, S., Orsy, P., Horvath, B., Maser-Gluth, C., Greiner, E., et al. (2008). G12-G13-LARG-mediated signaling in vascular smooth muscle is required for salt-induced hypertension. Nat Med 14, 64-68.

Woo, W. M., Zhen, H. H., and Oro, A. E. (2012). Shh maintains dermal papilla identity and hair morphogenesis via a Noggin-Shh regulatory loop. Genes Dev 26, 1235-1246.

Wu, S., Wu, Y., and Capecchi, M. R. (2006). Motoneurons and oligodendrocytes are sequentially generated from neural stem cells but do not appear to share common lineage-restricted progenitors in vivo. Development 133, 581-590.

Wu, Z., Autry, A. E., Bergan, J. F., Watabe-Uchida, M., and Dulac, C. G. (2014). Galanin neurons in the medial preoptic area govern parental behaviour. Nature 509, 325-330.

Xiao, Y., Thoresen, D. T., Miao, L., Williams, J. S., Wang, C., Atit, R. P., Wong, S. Y., and Brownell, I. (2016). A Cascade of Wnt, Eda, and Shh Signaling Is Essential for Touch Dome Merkel Cell Development. PLoS Genet 12, e1006150.

Yazdabadi, A., Whiting, D., Rufaut, N., and Sinclair, R. (2012). Miniaturized Hairs Maintain Contact with the Arrector Pili Muscle in Alopecia Areata but not in Androgenetic Alopecia: A Model for Reversible Miniaturization and Potential for Hair Regrowth. Int J Trichology 4, 154-157.

Zahalka, A. H., Arnal-Estape, A., Maryanovich, M., Nakahara, F., Cruz, C. D., Finley, L. W. S., and Frenette, P. S. (2017). Adrenergic nerves activate an angio-metabolic switch in prostate cancer. Science 358, 321-326.

Zhang, B., and Hsu, Y. C. (2017). Emerging roles of transit-amplifying cells in tissue regeneration and cancer. Wiley Interdiscip Rev Dev Biol 6, e282.

Zhang, B., Ma, S., Rachmin, I., He, M., Baral, P., Choi, S., Goncalves, W. A., Shwartz, Y., Fast, E. M., Su, Y., et al. (2020). Hyperactivation of sympathetic nerves drives depletion of melanocyte stem cells. Nature 577, 676-681.

Zhang, B., Tsai, P. C., Gonzalez-Celeiro, M., Chung, 0., Boumard, B., Perdigoto, C. N., Ezhkova, E., and Hsu, Y. C. (2016). Hair follicles' transit-amplifying cells govern concurrent dermal adipocyte production through Sonic Hedgehog. Genes Dev 30, 2325-2338.

Zhang, Y. V., Cheong, J., Ciapurin, N., McDermitt, D. J., and Tumbar, T. (2009). Distinct self-renewal and differentiation phases in the niche of infrequently dividing hair follicle stem cells. Cell Stem Cell 5, 267-278.

Zurborg, S., Piszczek, A., Martinez, C., Hublitz, P., Al Banchaabouchi, M., Moreira, P., Perlas, E., and Heppenstall, P. A. (2011). Generation and characterization of an Advillin-Cre driver mouse line. Mol Pain 7, 66.

### Example 2

### Evaluating and Enhancing AAV-Mediated Strategies to Treat Skin Diseases

Treatment of most skin conditions currently relies on the development of compounds or chemicals that can manipulate specific pathways. This approach has limited scope for specific diseases and is not cell-type specific. The approach described herein differs in that it can be applied to modifying genes and pathways with endless possibilities. AAV serotypes and enhancer/promoter combinations will be defined that can achieve cell-type-specific delivery and expression in diverse skin cells. In addition, the potential of using local delivery of AAVs to treat skin diseases will be tested and demonstrated. This will demonstrate the rapid translation of gene/pathway discovery in skin biology to the treatment of diseases.

### To Establish Methods for Cell-Type-Specific Gene Modification in a Wide Variety of Skin Cell Populations

Skin serves as a physical barrier protecting organisms from injury, infection, and dehydration. Skin also regulates body temperature and receives complex sensory inputs. These diverse functions are made possible by a rich array of cell types. The epidermis, the hair follicle, and the melanocyte lineage contain tissue-resident stem cells and are among some of the most highly regenerative tissues in adult mammals. These stem cells regenerate in a rich environment filled with fibroblasts, immune cells, neurons, blood vessels, muscle, and adipocytes. Mutations in these various cell types or dysregulations of these cell-cell interactions lead to diseases and conditions such as hair loss, hair graying, delayed wound healing, blistering diseases, loss of sensation, and diverse skin cancers including melanoma and basal cell carcinoma2.

AAV-mediated gene therapy has enormous potential in treating a wide spectrum of skin diseases. Preliminary data shows that local delivery of AAV8 through intradermal injection is a promising strategy by which to achieve skin-specific gene delivery. Although this relatively broad infectivity can already provide useful application for certain skin diseases, discovering strategies to enhance cell-type specificity can expand the utility of AAV when cell-type specificity is desired (e. g., correct mutations or express transgenes or toxins only in a specific cell type). The aim is to identify approaches that can introduce AAVs into diverse cell types in skin in a cell-type specific manner.

Various serotypes of AAVs will be explored. A wide variety of CAG-EFP containing AAVs (AAV1 to AAV9, AAV-DJ, AAV-Php.s) will be tested to determine their cell-type specificity in the skin. FACS and immunofluorescence will be conducted to determine if different AAVs have differential infectivity in epidermal stem cells, hair follicle stem cells, melanocyte stem cells, diverse dermal fibroblasts, nerve fibers, Schwann cells, blood vessels, and immune cells in skin.

Cell-type-specific promoters/enhancers will be defined. For example, several cell-type-specific genes have been identified for various cell types in skin, including for hair follicle stem cells, epidermal stem cells, and dermal papilla—three skin cell types that are important for hair follicle regeneration and wound healing²⁻⁶. Candidate enhancer elements will be cloned from cell type specific transcription factors and their ability to drive reporter gene expression will be tested in a cell-type-specific manner. In addition, novel single cell profiling (a new method that allowed us to conduct single cell RNAseq and single cell ATACseq within the same cell) was recently conducted for the whole skin⁷, which provided a powerful new way to predict regulatory elements that can drive gene expression in a cell type-specific manner at the single cell level. Candidate regulatory elements will be designed based on this newly obtained profiling data and parallel in vivo enhancer assays will be conducted to screen and identify DNA elements that can drive gene expression in a cell-type specific manner in skin⁸⁻⁹.

Testing the efficacy of using AAV-mediated gene delivery in treating skin diseases To test the feasibility of using AAV as a therapeutic strategy for treating skin diseases, three distinct problems to tackle were identified: hair follicle regeneration, wound healing, and epidermolysis bullosa. Hair loss occurs in many situations, including male pattern baldness, chronic stress, and chemotherapy. In all conditions, hair follicle stem cells remain, but stay quiescent¹⁰. The importance of SHH and Gas6 was previously identified. Wound healing is a complex process that involves essentially all of the cell types in skin. It has been discovered that SHH can affect multiple cell types concurrently to promote wound repair¹⁰. While gene correction in cultured keratinocytes and regrafting of these corrected keratinocytes back to patient has been used with some clinical success to treat epidermolysis bullosa¹¹⁻¹², this is an extremely laborious, expensive, and painful procedure to go through with lengthy recovery. Direct gene editing in vivo can provide a rapid and simple alternative.

For hair follicle regeneration, AAVs that carry SHH or Gas6 will be introduced during the telogen (resting) phase of the hair cycle. The hair growth speed of SHH-, Gas6-, and GFP- injected mice will be compared in control and stressed situation to see which factor(s) are effective in promoting hair growth

To promote wound healing, a full-thickness biopsy wound on the backs of the mice will be created, followed by intradermal injections containing CAG-SHH expressing AAV8. The wound-healing speed of CAG-GFP- and CAG-SHH-injected skin will be compared.

To correct epidermolysis bullosa, two approaches will be assessed: (a) exon skipping by excising the mutated exon80 using gRNAs that will result in the deletion of exon80¹³, and (b) a direct base conversion from A (mutant base pair) to G (wildtype base pair) using AAVs containing an adenine base editor14.

### REFERENCE

1. Samulski, R. J. & Muzyczka, N. AAV-Mediated Gene Therapy for Research and Therapeutic Purposes. Annu Rev Virol 1, 427-451 (2014).
2. Hsu, Y. C., Li, L. & Fuchs, E. Emerging interactions between skin stem cells and their niches. Nat Med 20, 847-856 (2014).
3. Hsu, Y. C., Li, L. & Fuchs, E. Transit-amplifying cells orchestrate stem cell activity and tissue regeneration. Cell 157, 935-949 (2014).
4. Ge, Y., et al. Stem Cell Lineage Infidelity Drives Wound Repair and Cancer. Cell 169, 636-650 e614 (2017).
5. Adam, R. C., et al. Pioneer factors govern super-enhancer dynamics in stem cell plasticity and lineage choice. Nature 521, 366-370 (2015).
6. Rendl, M., Lewis, L. & Fuchs, E. Molecular dissection of mesenchymal-epithelial interactions in the hair follicle. PLoS Biol 3, e331 (2005).
7. Ma, S., et al. Chromatin-mediated lineage priming and chromatin potential identified by shared single cell profiling of RNA and chromatin. Nature, Submitted(2019).
8. White, M. A., Myers, C. A., Corbo, J. C. & Cohen, B. A. Massively parallel in vivo enhancer assay reveals that highly local features determine the cis-regulatory function of ChIP-seq peaks. Proc Natl Acad Sci U S A 110, 11952-11957 (2013).
9. Shen, S. Q., et al. Massively parallel cis-regulatory analysis in the mammalian central nervous system. Genome Res 26, 238-255 (2016).
10. Zhang, B., et al. Hair follicles' transit-amplifying cells govern concurrent dermal adipocyte production through Sonic Hedgehog. Genes Dev 30, 2325-2338 (2016).
11. Webber, B. R., et al. CRISPR/Cas9-based genetic correction for recessive dystrophic epidermolysis bullosa. NPJ Regen Med 1(2016).
12. Hirsch, T., et al. Regeneration of the entire human epidermis using transgenic stem cells. Nature 551, 327-332 (2017).
13. Wu, W., et al. Efficient in vivo gene editing using ribonucleoproteins in skin stem cells of recessive dystrophic epidermolysis bullosa mouse model. Proc Natl Acad Sci USA 114, 1660-1665 (2017).
14. Gaudelli, N. M., et al. Programmable base editing of A*T to G*C in genomic DNA without DNA cleavage. Nature 551, 464-471 (2017).

### Example 3

### Cell Type-Specific Transduction in Skin Using Adeno-Associated Virus (AAV) As Delivery Vector

The hair follicle cycles between growth, regression, and rest phases. This cyclic activity is regulated by the activation and quiescence of hair follicle stem cells (HFSCs). Recent studies indicate that the crosstalk between HFSCs and the stem cell niche play an important role in regulating hair follicle maintenance. Due to the complex architecture and diverse skin cell types, it is often challenging to study how a specific cell type of interest influences the microenvironment of HFSC niche. There is a necessity for developing an investigation tool which can induce cell-type specific transduction in skin.

In the study described herein, AAVs were incorporated with a reporter transgene and injected intradermally on the back skin of mice. Their transduction efficiency was evaluated via fluorescence microscopy and the labeled cells quantified. In addition, immunohistochemistry was performed with different skin cell markers to identify which skin cell types were infected by the AAV candidates.

The pattern of AAV transduction in skin was shown to vary depending on capsid serotype, promoter, and the timing of injection. In adult mice, AAV8-CAG-GFP showed the most widespread transduction, while AAV6-CAG-GFP and AAV-PHP. S-EF1a targeted arrector pili muscle with a high frequency. The P0 injection of AAV in neonatal mice showed a significant improvement in transduction efficiency, compared to the adult injection. Importantly, the P0 injection of AAV6-EF1a-DTA-mCherry was highly efficient to transduce APM. In addition, the P0 injection of AAV-PHP. S-EF1a-DTA-mCherry showed tissue-specific transduction in dermal papilla.

This study demonstrated that the combination of different conditions can achieve a cell-type specific transduction of AAV in mice skin. With a blend of conditions, AAV can induce cell-type-specific transduction in the HFSC niche, including dermal fibroblasts, adipocytes, APM and DP.

### Results

### Capsid Serotype Influences AAV Transduction in Mice Skin

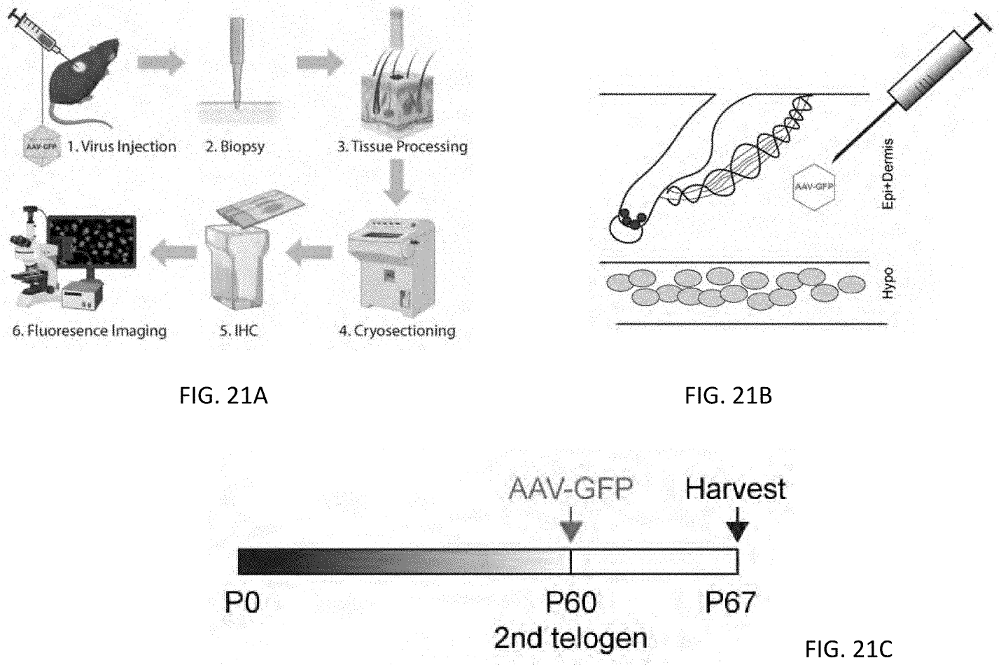

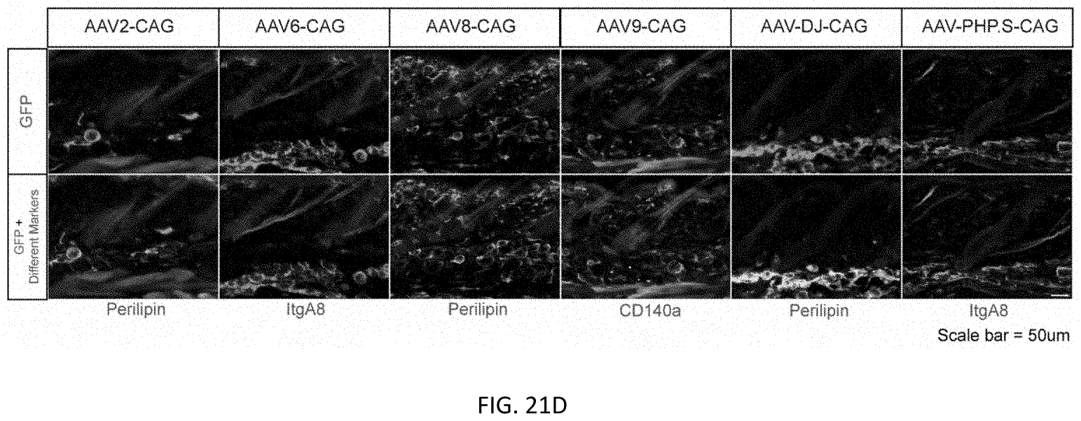

To optimize the AAV administration in skin, the transduction efficiency of different AAV serotypes was evaluated. The transduction requires successful entry into the host cell, and the success rate of viral entry can depend on how the AAV capsid interacts with the receptors on the target cell. Therefore, it was hypothesized that AAV serotypes bearing different capsid proteins would result in varying transduction patterns in the skin. To examine this, different AAV serotypes were injected in adult mice and the transduction in skin was evaluated via immunohistochemistry (FIG. 21A). Previous studies reported that intravenous and retro-orbital injection delivered AAV via a systemic route and could lead to undesired infection in random tissues. Therefore, the present study utilized intradermal injection to maximize the local infection in the skin (FIG. 21B). The tested serotypes (AAV2, AAV6, AAV8, AAV9, AAV-DJ, and AAV-PHP. S) were combined with a CAG promoter and designed to induce the expression of the GFP reporter gene in the transduced cells. The skin samples were harvested 7 days post injection (P67) and were analyzed via immunohistochemistry (FIG. 21C). In fluorescence microscopy, Cy5 beads showed where the virus injection took place and reporter expression showed which cells were transduced by AAV injection.

The data suggested that different AAV serotypes showed varying transduction patterns (FIG. 21D). Each of the tested serotypes, AAV2, AAV6, AAV8, AAV9, AAV-DJ, and AAV-PHP. S, resulted in transducing dermal adipocytes. GFP expressing adipocytes were evident throughout all of the skin samples, suggesting that dermal adipocytes can be transduced with most serotypes. The target-specificity, however, varied among the serotypes. AAV2-CAG-GFP and AAV-DJ-CAG-GFP showed minimal transduction pattern where the infection appeared to be limited primarily to adipocytes, and only minimal infection of other skin cell types. In contrast, AAV6-CAG-GFP and AAV-PHP. S-CAG-GFP seemed highly efficient in infecting APM. These observations were confirmed by conducting co-immunohistochemistry with Perilipin, the adipocyte marker and ItgA8, the APM marker. Among the tested serotypes, AAV8-CAG-GFP and AAV9-CAG-GFP showed the most widespread infection pattern, transducing a large number of dermal fibroblasts. The dermal fibroblasts transduction was confirmed as the co-localizations of CD140a, the dermal fibroblast marker, and GFP were frequently detected. Collectively, these data indicate that the capsid serotypes influence the transduction abilities of AAV. Importantly, some AAV serotypes transduce specific skin cell types allowing a user to choose the most appropriate capsid to target a cell of interest.

### Timing of Injection Influences AAV Transduction in Mice Skin

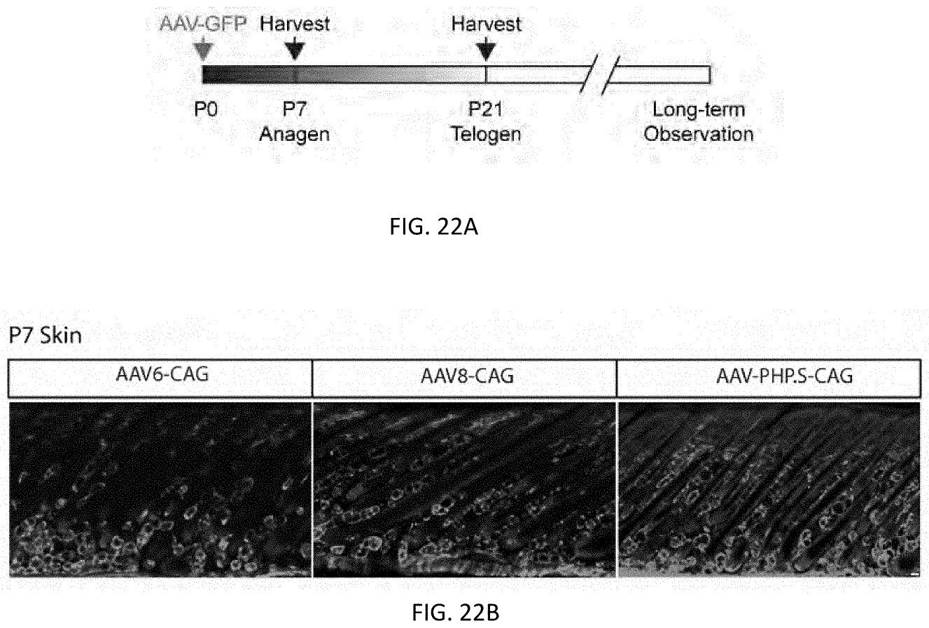

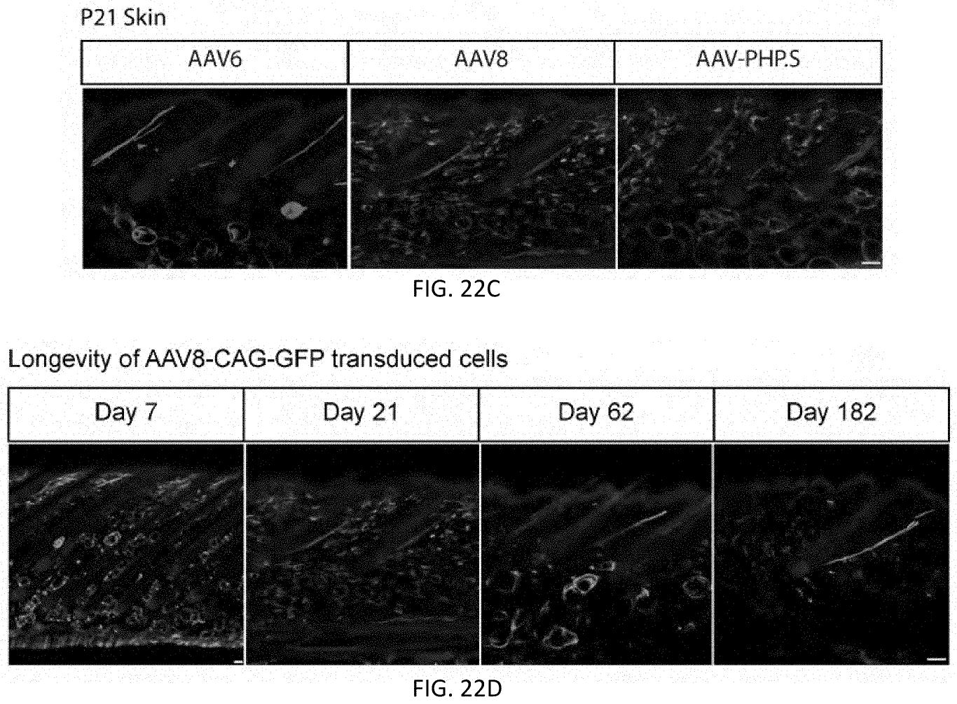

Whether the timing of injection will have an effect on the transduction abilities of AAV was then evaluated. It was hypothesized that AAV capsid protein may be capable of entering more diversified cell types in developing mice before the skin cells are differentiated and compartmentalized. To assess this question, three serotypes, AAV6, AAV8, and AAV-PHP. S which showed most distinctive patterns of transduction, were tested following intradermal injection in mice on neonatal day P0 (FIG. 22A). In view of the smaller body size of mice, the amount of virus solution used for injection was decreased to 20u1 (total 2xE10 genomic copies of AAV). P0 injected mice were biopsied at day 6 and 21. Additionally, the animals were monitored long-term to evaluate the longevity of the transduced cells. The skin samples were analyzed via immunohistochemistry staining.

Overall, P0 injection of the three serotypes resulted in widespread and robust transduction despite the lesser amount of virus given. The P0 injected mice of AAV8 showed a successful transduction of arrector pili muscle, adipocyte, and dermal fibroblast in P7 developmental stage (FIG. 22B). A large number of GFP expressing cells were evident in the P21 adult stage, and the total number of transduced cells seemed to be increased, compared to that observed in the adult injection (FIG. 22C). The P0 injected mice of AAV6 and AAV-PHP. S showed similar transduction patterns as compared to when they were injected in adults. These mice showed an increased number of GFP expressing cells, while APM still appeared to be the primary target. The quantification data showed that 78% of APM (32 out of 41) was transduced in serotype 6, while 88% of APM (45 out of 51) was transduced in serotype PHP. S. Collectively, these data suggest that the timing of injection influences the transduction abilities of AAV, and the choice of injection time point is an important factor for optimizing AAV administration.

### AAV Transduction in Skin Lasts Over 6 Months

The longevity of AAV-transduced cells was evaluated by quantifying GFP expressing cells over the long term. To examine this, one mouse of the P0 injected cohort of AAV8-CAG-GFP was undertaken for biopsies at multiple time points, day 7, 21, 62, and 182. The biopsy skin was then stained for GFP to observe the presence of AAV-transduced cells. The immunohistochemistry staining showed that AAV-transduced cells persist up to 6 months but the GFP signaling gradually faded away in adipocytes and dermal fibroblasts (FIG. 22D). This phenomenon may be explained by the natural turnover of the skin. The AAV vectors do not integrate into the genome of host cells remaining episomal, and thus diluted with subsequence cellular division. As a result, the AAV transduced cell population decreases over time. However, strong GFP signaling was detected in APM, suggesting that the transduced state may last longer in the non-proliferative cell types. These data demonstrate that AAV-mediated transgene expression can last over 6 months in skin.

### Combination of AAV-PHP. S and EF1a Promoter Showed Tissue-Specific Transduction in APM and DP

In addition to capsid serotype and the timing of injection, it was hypothesized that the regulatory elements in AAV transgene expression cassettes will have an effect on transduction abilities. The serotypes previously tested were incorporated with CAG promoter upstream of the GFP expression cassette. To test the hypothesis, AAV serotypes were incorporated with the EF1a promoter and were injected at P0. The data obtained was compared to those AAV serotypes with the CAG promoter. To label the transduced cells, the red fluorescent protein (RFP or mCherry) expression cassette was inserted downstream of the EF1a promoter. All of the tested AAV serotypes with EF1a promoter contained Cre-dependent DTA transgene, a cell death-inducing gene cassette which only becomes activated with the presence of Cre molecule.

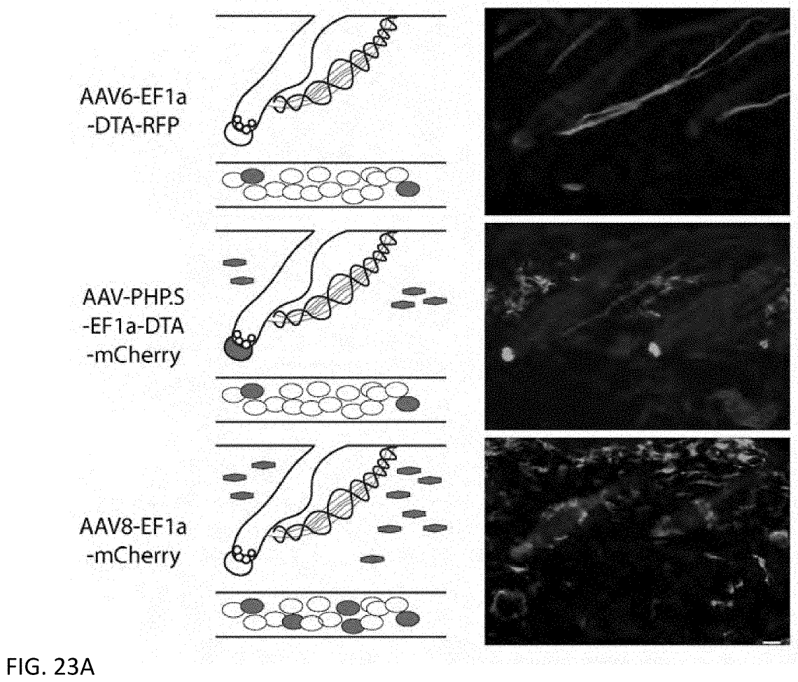

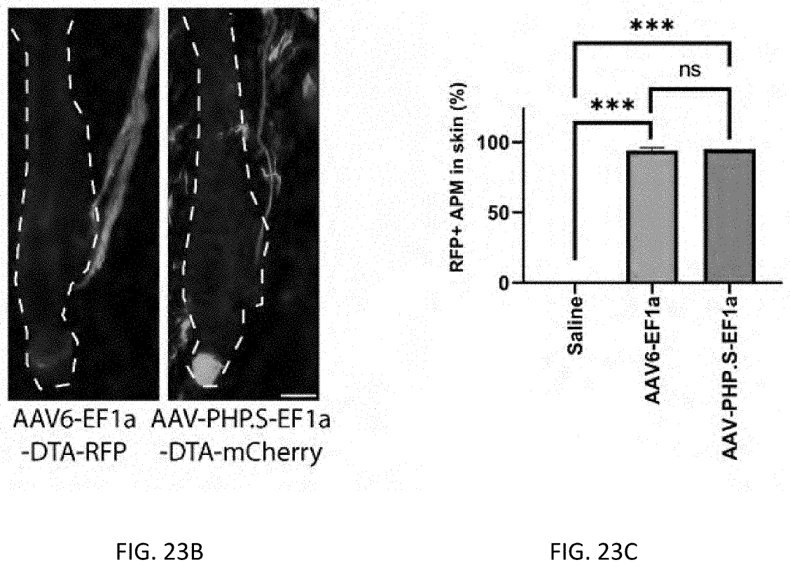

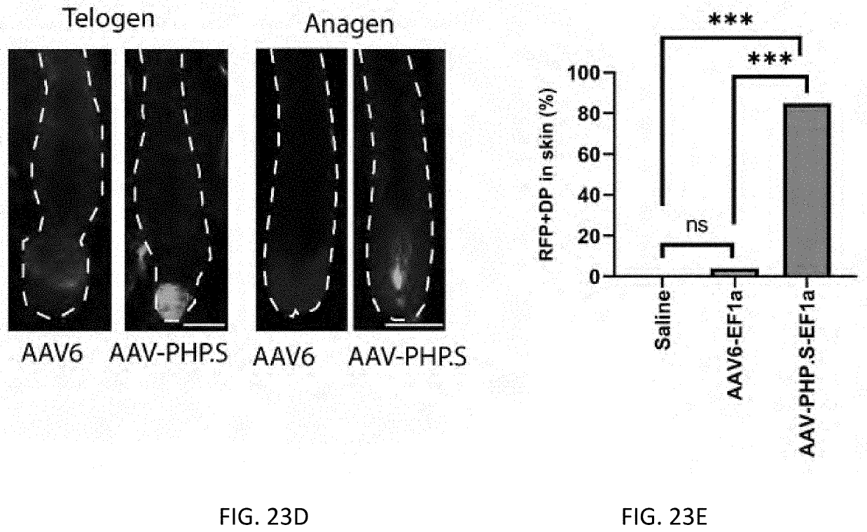

The RFP antibody staining demonstrated that the use of EF1a promoter influenced the distribution and transduction pattern of AAV when analyzed at P21. All of the AAVs with the EF1a promoter that were tested showed a reduced frequency of transduction in adipocytes when compared to AAVs with the CAG promoter (FIG. 23B). Importantly, AAV6-EF1a-DTA-RFP showed a strong transduction in APM with a reduced transduction in adipocytes. This result led to a tissue-specific transduction of APM in AAV6-EF1a- DTA-RFP. In addition, the use of the EF1a promoter resulted in an increase in the transduction efficiency in APM. Quantification of RFP signaling demonstrated that 94% of APM were transduced in AAV6-EF1a-DTA-mChery (49 out of 52) and 95% in AAV-PHP. S-EF1a-DTA-mCherry (69 out of 73) (FIGS. 23B-23C). Collectively, the data suggest that P0 injection of AAV6-EF1a-DTA-RFP and AAV-PHP. S-DTA-mCherry are highly efficient for transducing APM.

In addition, P0 injection of AAV-PHP. S-EF1a-DTA-mCherry showed a unique infection pattern, transducing APM and dermal papilla (DP). This result is interesting in view of a previous study by Hengge et al. which reported that neonatal injection of AAVlacZ led to transduction in epidermal keratinocytes and HF epithelial cells, but did not show DP infection (Hengge & Mirmohammadsadegh, 2000). Quantification data showed that 85% of DP were expressing RFP in AAV-PHP. S-EF1a-DTA-mCherry (FIGS. 23D-23E). This finding is significant when studying DP, which may have a close relationship with the HFSC niche population. P0 injection of AAV-PHP. S-EF1a-DTA-mCherry appears promising in transducing both DP and APM and can serve as a tool for studying how these cell types influence the microenvironment of HFSC niche.

### Application of Targeted Transduction of AAV in Mice Skin

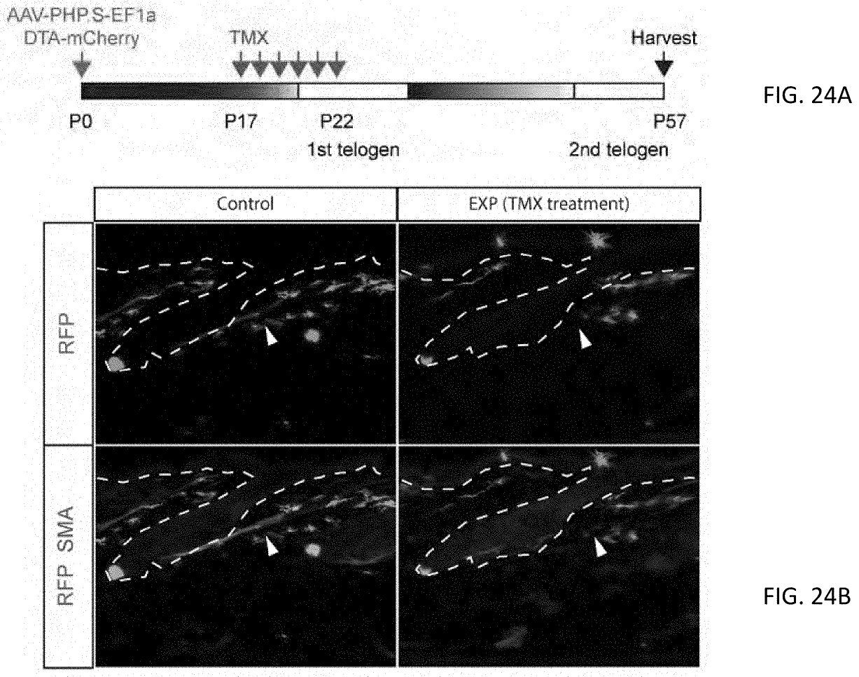

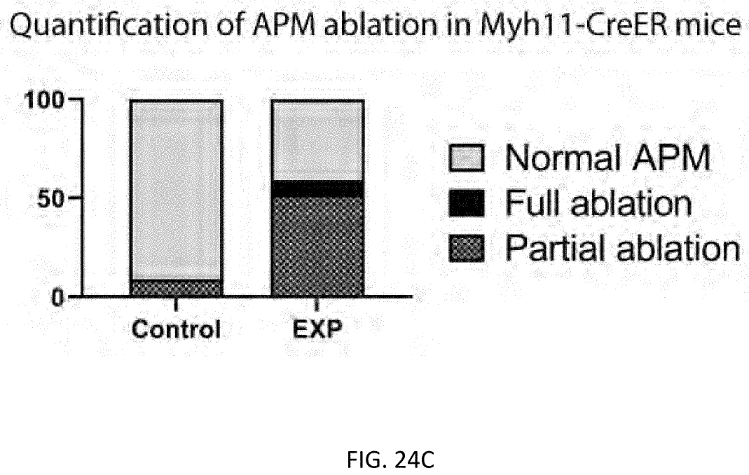

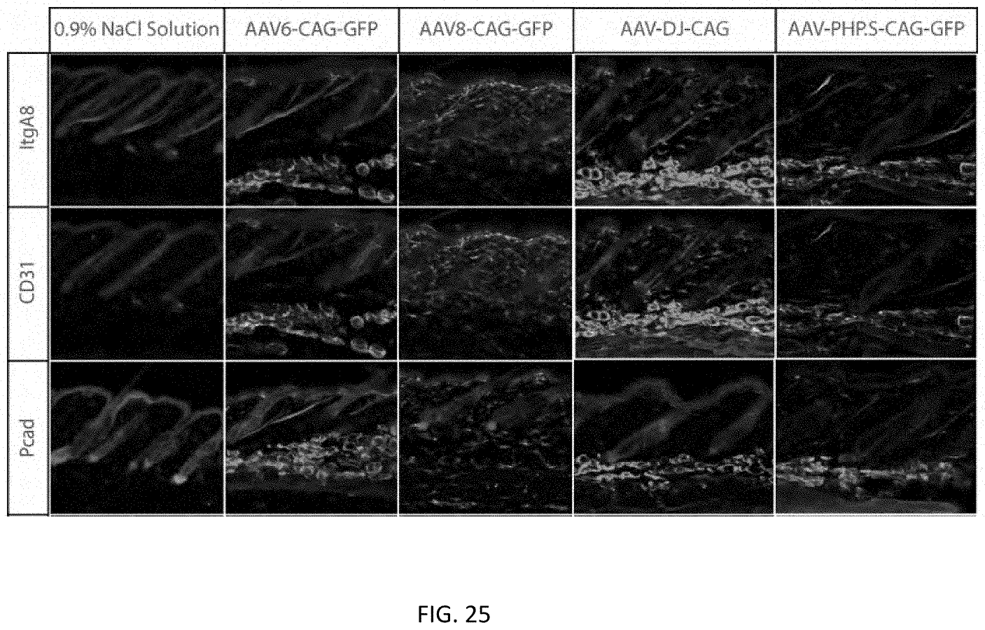

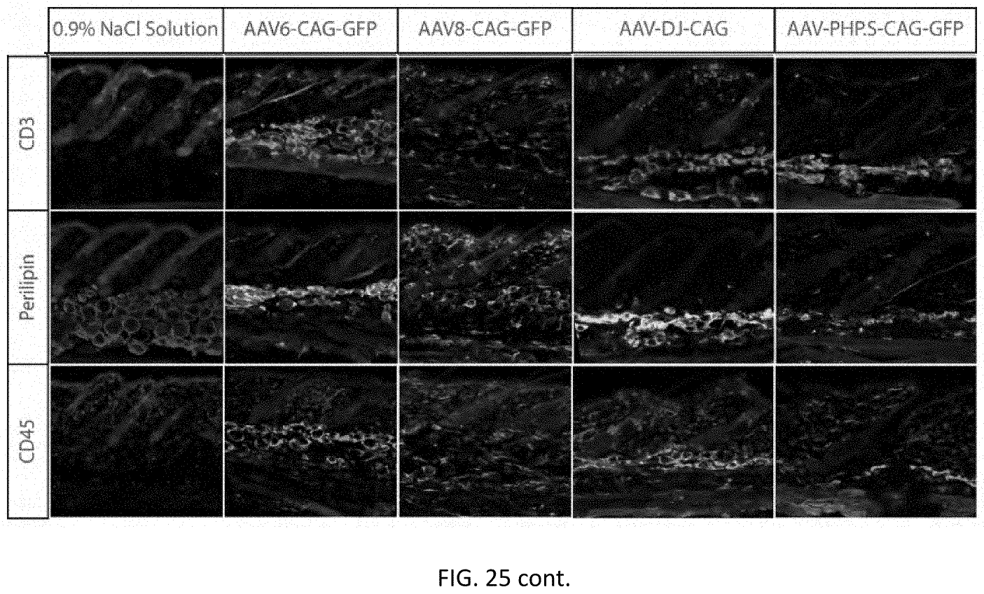

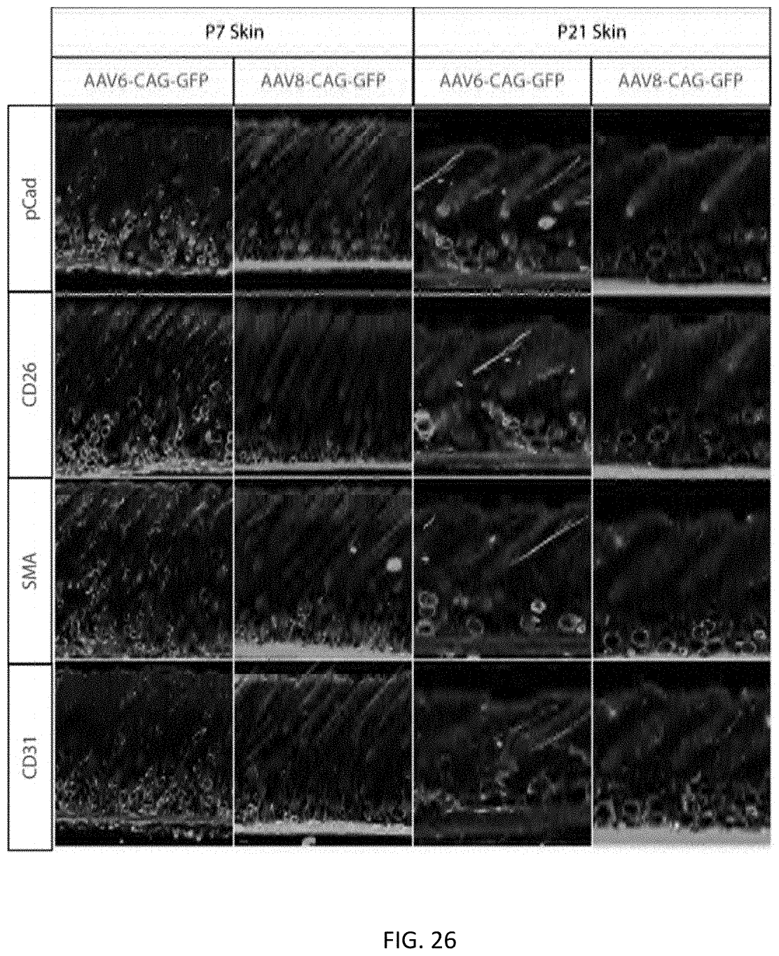

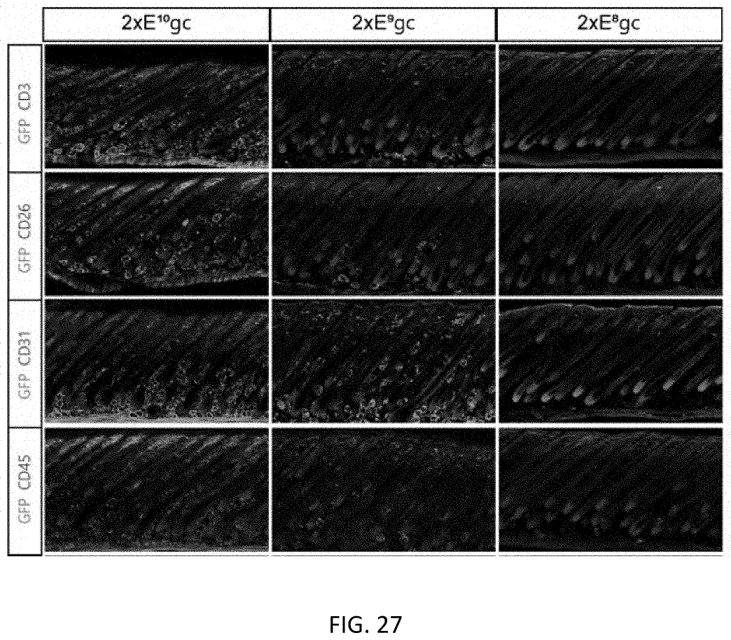

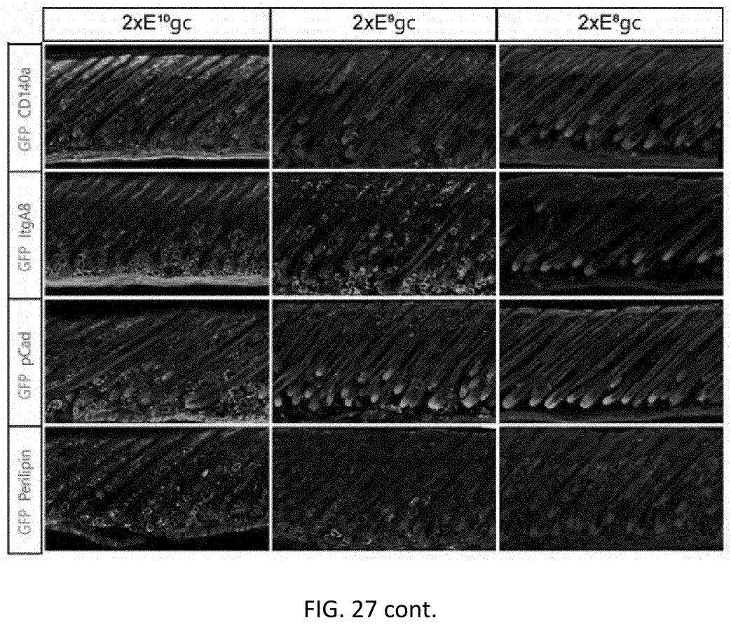

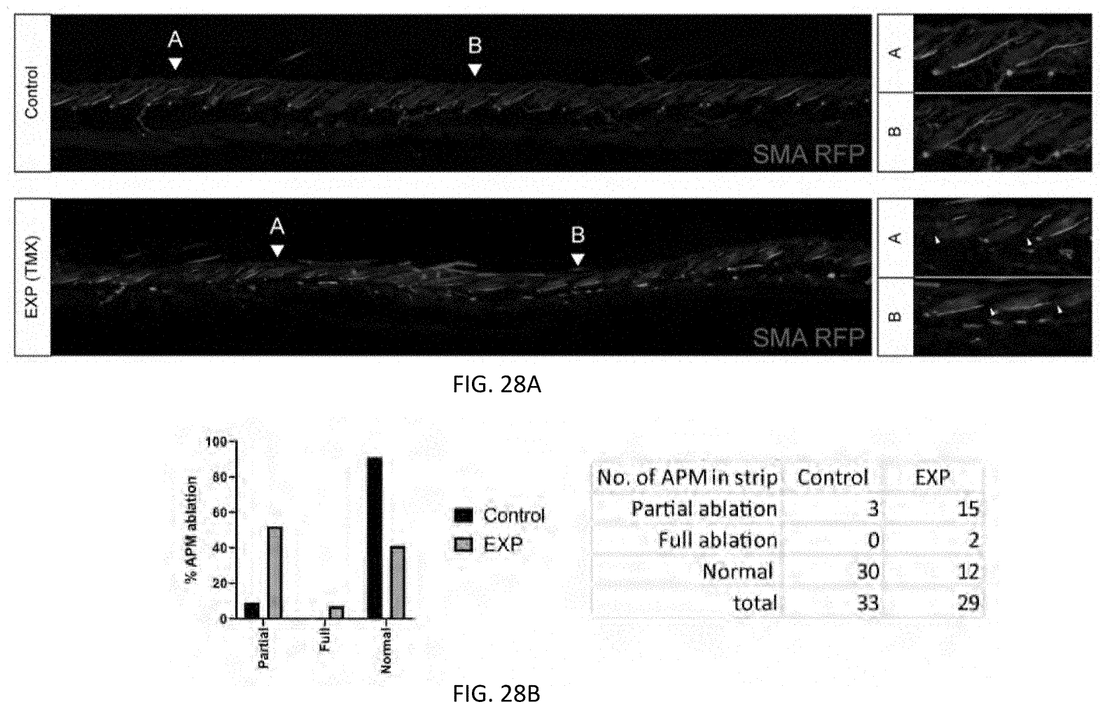

Modifications to the capsid serotype, promoter, and the timing of injection have shown effects to tissue-specific transduction in some skin cell types. In particular, P0 injection of AAV-PHP. S-EF1a-DTA-mCherry resulted in outstanding transduction efficiency in APM and DP. Given this finding, it was investigated whether the ablation of APM has an effect on the maintenance of HF. The P0 injection of AAV-PHP. S-EF1a-DTA-mCherry was performed in Myh11-CreER transgenic mice, following 6 rounds of tamoxifen treatment from P17 to P22. The P0 injected cohort of AAV-PHP. S. -DTA-mCherry was divided into control and experimental groups (n=2, each group). The control group did not receive further treatment, while the experimental group was treated with tamoxifen. These mice were observed and harvested in the next following hair cycle (FIG. 24A). The P0 injection induced the flex-DTA-mCherry transgene in the transduced cells. It was shown that diverse skin cell types, including APM, dermal fibroblasts and adipocytes, were infected by AAV. The DTA transgene, however, was only activated in APM and ensured the targeted ablation. In Myh11-CreER transgenic mice, the expression of Cre is tissue-specific under control of smooth muscle (Mhy11) promoter, and the Cre-dependent DTA transgene is activated in APM exclusively.

As a result, P0 injection of AAV-PHP. S-EF1a-DTA-mCherry was able to induce the targeted ablation of APM in Myh11-CreER mice treated with tamoxifen treatment. Immunofluorescent staining for RFP showed that P0 injection successfully transduced APM, 97% (33 out of 34) in the control group and 94% (29 out of 31) in the experimental group. Then, the co-localization of RFP signal and smooth muscle actin (SMA) antibody was observed to quantify APM ablation. Whereas the control group showed normal APM, the experimental group frequently showed a discontinuous muscle fiber in arrector pili which indicates the partial or full ablation of APM (FIG. 24B). In the control group, 9% of total APM (3 out of 33) showed discontinuous muscle fiber in arrector pili, which might be due to technical reasons such as section angle. In the experimental group, 54% of total APM (15 out of 29) were partially ablated and 7% (2 out of 29) were fully ablated resulting in HFs with no attached APM (FIG. 24C).

However, the experimental group with partially or even fully ablated APM did not show a marked difference in the timing of hair cycle entry. It is possible the remaining APM are still functional and rescue the animals from phenotype. It is also possible that APM may play a significant role in developing pups, rather than in adult mice. Collectively, these data demonstrate the usage of AAV to induce targeted-cell death in specific tissues, showing AAV's potential in various therapeutic applications.

### Discussion

To date, a number of studies have utilized AAV vectors as tools for gene delivery. In one study, AAV-mediated gene therapy was used for ocular gene transfer, which rescued blindness in aged animals and the effect was sustained long-term (Liu et al., 2018). While AAV holds value in various therapeutic strategies, understanding the tissue-tropism of AAV and preventing undesired infection of AAV remains an overarching goal for the field (Hickey et al., 2017). It would be beneficial to establish a system in which AAV can target specific cell types of interest.

In this study, the optimization of AAV mediated transgenesis in mice skin was demonstrated. The pattern of AAV transduction in skin varied depending on capsid serotype, promoter, and the timing of injection. In adult mice, AAV8-CAG-GFP showed the most widespread transduction, while AAV6-CAG-GFP and AAV-PHP. S-EF1a targeted APM with a high frequency. The AAV injection in P0 neonatal mice showed a significant improvement in transduction efficiency. Importantly, the P0 injection of AAV6-EF1a-DTA-mCherry was highly efficient to transduce APM. The P0 injection of AAV-PHP. S-EF1a-DTA-mCherry also showed tissue-specific transduction in APM and DP. Finally, these findings were used to achieve targeted-cell death in APM. The P0 injection of AAV-PHP. S-EF1a-DTA-mCherry in Mhy11-CreER mice resulted in the partial ablation in 58% of APM and full ablation in 7% of APM.

This study demonstrated that the combination of different conditions can achieve cell-type specific expression of AAV in mice skin. The comparison of different injection time points also suggest that AAV-mediated gene delivery may show varying efficiency in adult and juvenile mice. In addition, the tracing of GFP expressing cells in P0 injected mice provides an approximation of how long the effect of AAV-mediated gene transfer will last in clinical trials.

### Materials and Method

### AAV Construction and Preparation

Plasmid transformation was adapted from High Efficiency Transformation Protocol (C30401). A 30 ul aliquoted tube of NEB Stable Competent E. Coli cells was thawed on ice until the last ice crystals disappear. The volume of lul containing 100 ng of plasmid DNA was added to the 30 ul of E. Coli cells. Then the cell mixture was cultured in 570 ul of NEB 10-beta/Stable Outgrowth Medium at 30° C. for 60 minutes. The cell mixture was evenly spread onto an ampicillin selection plate and incubated at 37° C. overnight. Purification of the plasmid DNA was performed using ZymoPure II™ Plasmid Midiprep kit (D4200). The purified plasmid DNA was measured with Nanodrop and transferred to sterile tube. The tube of concentrated DNA was then shipped to Welgen, the AAV construction company, to be packaged with a desired AAV serotypes.

Prior to injection, the AAV plasmids were diluted to the volume of 40 ul with the concentration of 1×E12 gc/ml with the sterile sodium chloride solution, 0. 9% in water, (Sigma-Aldrich, S8779), that is 4×E1° genomic copies of AAV. Added with lul of Cy5 beads ThermoFisher, F8807), a total 41 ul of virus solution was administrated intradermally on back skin of adult mice (P60).

All tested AAV vectors incorporated either the chicken beta-Actin (CAG) promoter or elongation factor-1 alpha (EF1a) promoter. Downstream of the promoter domain, the reporter transgenes (GFP, RFP and mCherry) cassettes were inserted to label the transduced cells.

### Mice

All husbandries and procedures involving animal subjects were performed upon the approval by Harvard University Institutional Animal Care and Use Committee (IACUC) and conducted in accordance with NIH guidelines. C57BL6/J female mice (Catalog #000664) were purchased from the Jackson Laboratory. All animals were housed up to five to a cage and maintained with food and water with a 12 hours light/dark cycle.

### Intradermal Injection

The prepared AAV mixture was injected intradermally on the back skin of mice, using 31G Insulin syringe (BD #328438). In adult injection, the mice were anesthetized with isoflurane and injected with total 4×E¹⁰ genomic copies of AAV. The skin samples were harvested after 6 days post injection. In P0 injection, the newborn pups were anesthetized with ice for 3 minutes and injected with total 2×E¹⁰ genomic copies of AAV. The time of newborn delivery was monitored closely and P0 pups were injected as close as possible to their birth (0-12 hours postnatal). The skin samples were harvested at multiple time points (Day 6, 21, 62, and 182).

### Immunohistochemistry

The following antibodies were used in this study: ItgA8 (goat, R&D Systems AF4076-SP 1:100), Perilipin-1 (goat, Abcam 1:400), GFP (rabbit, Abcam ab290, 1:500), GFP (chicken, Ayes Labs, GFP-1010, 1:500), tdTomato (rat, KERAFAST EST203, 1:500), RFP (rabbit, Abcam ab61682, 1:500), SMA (mouse, Santa Cruz sc-32251, 1:100), CD3 (Thermo Fisher Scientific 14-0032-82, 1:100), CD31 (rat, eBioscience, 1:50), CD26 (goat, R&D Systems AF954-SP, 1:100), CD140a (goat, R&D Systems AF1062-SP, 1:100), P-Cad (goat, R&D Systems, AF761, 1:200), Perilipin-1 (goat, Abcam ab61682, 1:400)

### Histology

Skin samples were harvested, fixed in 4% paraformaldehyde (VWR #15710), embedded in Tissue-Tek OCT Compound (Sakura #4583) and cryo-sectioned with typically 40-60 um thickness. The frozen sections were fixed again for 2 minutes and undergone immunohistochemistry staining with different skin cell type markers. The sections were incubated with primary antibody at 4° C. overnight, followed by the secondary antibody staining at room temperature for 1 hour. Finally, the samples are mounted with Prolong Gold with DAPI (SouthernBiotech 0100-20) and preserved in 4° C.

### Fluorescent Microscopy

Immunofluorescent images were acquired with a Keyence epifluorescence microscope (Keyence America, BX-700) and imported into BZ-Analysis for Z-plane-stacked analysis. Images were further processed and assembled into panels using Image J, Adobe Photoshops CC 2019 and Adobe Illustrator 2019.

### REFERENCES

Alcolea, M. P., & Jones, P. H. (2014). Lineage analysis of epidermal stem cells. Cold Spring Harb Perspect Med, 4(1), a015206. doi:10.1101/cshperspect.a015206

Ariyachet, C., Tovaglieri, A., Xiang, G., Lu, J., Shah, M. S., Richmond, C. A., . . . Zhou, Q. (2016). Reprogrammed Stomach Tissue as a Renewable Source of Functional beta Cells for Blood Glucose Regulation. Cell Stem Cell, 18(3), 410-421. doi:10.1016/j. stem.2016.01.003

Cheng, C. C., Tsutsui, K., Taguchi, T., Sanzen, N., Nakagawa, A., Kakiguchi, K., . . . Fujiwara, H. (2018). Hair follicle epidermal stem cells define a niche for tactile sensation. Elife, 7. doi:10.7554/eLife.38883

Cheng, J. B., Sedgewick, A. J., Finnegan, A. I., Harirchian, P., Lee, J., Kwon, S., . . . Cho, R. J. (2018). Transcriptional Programming of Normal and Inflamed Human Epidermis at Single-Cell Resolution. Cell Rep, 25(4), 871-883. doi:10.1016/j.celrep.2018. 09. 006

Colella, P., Ronzitti, G., & Mingozzi, F. (2018). Emerging Issues in AAV-Mediated In Vivo Gene Therapy. Mol Ther Methods Clin Dev, 8, 87-104. doi:10.1016/j.omtm.2017.11.007

Driskell, R. R., Giangreco, A., Jensen, K. B., Mulder, K. W., & Watt, F. M. (2009). Sox2-positive dermal papilla cells specify hair follicle type in mammalian epidermis. Development, 136(16), 2815-2823. doi:10.1242/dev.038620

Duong, T. T., Lim, J., Vasireddy, V., Papp, T., Nguyen, H., Leo, L., . . . Mills, J. A. (2019). Comparative AAV-eGFP Transgene Expression Using Vector Serotypes 1-9, 7m8, and 8b in Human Pluripotent Stem Cells, RPEs, and Human and Rat Cortical Neurons. Stem Cells Int, 2019, 7281912. doi:10.1155/2019/7281912

Hanlon, K. S., Kleinstiver, B. P., Garcia, S. P., Zaborowski, M. P., Volak, A., Spirig, S. E., . . . Gyorgy, B. (2019). High levels of AAV vector integration into CRISPR-induced DNA breaks. Nat Commun, 10(1), 4439.doi:10.1038/s41467-019-12449-2

Heinrich, C., Spagnoli, F. M., & Berninger, B. (2015). In vivo reprogramming for tissue repair. Nat Cell Biol, 17(3), 204-211.doi:10.1038/ncb3108

Hengge, U. R. (2005). Progress and prospects of skin gene therapy: a ten year history. Clin Dermatol, 23(1), 107-114. doi:10.1016/j.clindermato1.2004.09.008

Hengge, U. R. (2006). Gene therapy progress and prospects: the skin—easily accessible, but still far away. Gene Ther, 13(22), 1555-1563. doi:10.1038/sj.gt.3302855

Hengge, U. R., & Mirmohammadsadegh, A. (2000). Adeno-associated virus expresses transgenes in hair follicles and epidermis. Mol Ther, 2(3), 188-194. doi:10. 1006/mthe.2000.0118

Hickey, D. G., Edwards, T. L., Barnard, A. R., Singh, M. S., de Silva, S. R., McClements, M. E., . . . MacLaren, R. E. (2017). Tropism of engineered and evolved recombinant AAV serotypes in the rdl mouse and ex vivo primate retina. Gene Ther, 24(12), 787-800. doi:10.1038/gt.2017.85

Hsu, Y. C., Li, L., & Fuchs, E. (2014). Transit-amplifying cells orchestrate stem cell activity and tissue regeneration. Cell, 157(4), 935-949. doi:10.1016/j.cell. 2014. 02. 057

Jahoda, C. A., & Christiano, A. M. (2011). Niche crosstalk: intercellular signals at the hair follicle. Cell, 146(5), 678-681. doi:10.1016/j.cell.2011.08.020

Kaufman, C. K., Zhou, P., Pasolli, H. A., Rendl, M., Bolotin, D., Lim, K. C., . . . Fuchs, E. (2003). GATA-3: an unexpected regulator of cell lineage determination in skin. Genes Dev, 17(17), 2108-2122. doi:10.1101/gad.1115203

Kurita, M., Araoka, T., Hishida, T., O'Keefe, D. D., Takahashi, Y., Sakamoto, A., . . . Izpisua Belmonte, J. C. (2018). In vivo reprogramming of wound-resident cells generates skin epithelial tissue. Nature, 561(7722), 243-247. doi:10.1038/s41586-018-0477-4

Lee, H., Koehler, D. R., Pang, C. Y., Levine, R. H., Ng, P., Palmer, D. J., . . . Hu, J. (2005). Gene delivery to human sweat glands: a model for cystic fibrosis gene therapy. Gene Ther, 12(24), 1752-1760. doi:10.1038/sj.gt.3302587

Liu, Y., Fortmann, S. D., Shen, J., Wielechowski, E., Tretiakova, A., Yoo, S., . . . Campochiaro, P. A. (2018). AAV8-antiVEGFfab Ocular Gene Transfer for Neovascular Age-Related Macular Degeneration. Mol Ther, 26(2), 542-549. doi:10.1016/j.ymthe.2017. 12. 002

Morgan, B. A. (2014). The dermal papilla: an instructive niche for epithelial stem and progenitor cells in development and regeneration of the hair follicle. Cold Spring Harb Perspect Med, 4(7), a015180. doi:10.1101/cshperspect.a015180

Naso, M. F., Tomkowicz, B., Perry, W. L., 3rd, & Strohl, W. R. (2017). Adeno-Associated Virus (AAV) as a Vector for Gene Therapy. BioDrugs, 31(4), 317-334. doi:10.1007/s40259-017-0234-5

Rompolas, P., Deschene, E. R., Zito, G., Gonzalez, D. G., Saotome, I., Haberman, A. M., & Greco, V. (2012). Live imaging of stem cell and progeny behaviour in physiological hair-follicle regeneration. Nature, 487(7408), 496-499. doi:10.1038/nature11218

Shi, W., Teschendorf, C., Muzyczka, N., & Siemann, D. W. (2002). Adeno-associated virus-mediated gene transfer of endostatin inhibits angiogenesis and tumor growth in vivo. Cancer Gene Ther, 9(6), 513-521. doi:10.1038/sj.cgt.7700463

Sujata, L., & Chaudhuri, S. (2008). Stem cell niche, the microenvironment and immunological crosstalk. Cell Mol Immunol, 5(2), 107-112. doi:10.1038/cmi.2008. 13

Tan, D. W., Jensen, K. B., Trotter, M. W., Connelly, J. T., Broad, S., & Watt, F. M. (2013). Single-cell gene expression profiling reveals functional heterogeneity of undifferentiated human epidermal cells. Development, 140(7), 1433-1444. doi: 10.1242/dev.087551

Torkamani, N., Rufaut, N. W., Jones, L., & Sinclair, R. D. (2014). Beyond goosebumps: does the arrector pili muscle have a role in hair loss? Int J Trichology, 6(3), 88-94. doi:10.4103/0974-7753.139077

Yokouchi, M., Atsugi, T., Logtestijn, M. V., Tanaka, R. J., Kajimura, M., Suematsu, M., . . . Kubo, A. (2016). Epidermal cell turnover across tight junctions based on Kelvin's tetrakaidecahedron cell shape. Elife, 5. doi:10.7554/eLife.19593

Zhang, B., Ma, S., Rachmin, I., He, M., Baral, P., Choi, S., . . . Hsu, Y. C. (2020). Hyperactivation of sympathetic nerves drives depletion of melanocyte stem cells. Nature, 577(7792), 676-681. doi:10.1038/s41586-020-1935-3

Zincarelli, C., Soltys, S., Rengo, G., & Rabinowitz, J. E. (2008). Analysis of AAV serotypes 1-9 mediated gene expression and tropism in mice after systemic injection. Mol Ther, 16(6), 1073-1080. doi:10.1038/mt.2008.76

### Example 4

### Using Adeno-Associated Viruses to Explore Novel Gene Function and Enhance Wound Repair Iin the Skin

The utility of an AAV delivery system in the skin for both research and therapeutic purposes has not been thoroughly explored. In this study, the cellular specificity of various AAVs' transduction patterns were identified. Moreover, by using SHH and Edn3 as a proof of concept, AAV was established as a valid gene delivery tool under homeostatic and pathological conditions for two purposes: discovering novel regulatory factors and therapeutically improving wound healing.

### Results

### Identifying AAV Infectivity Patterns

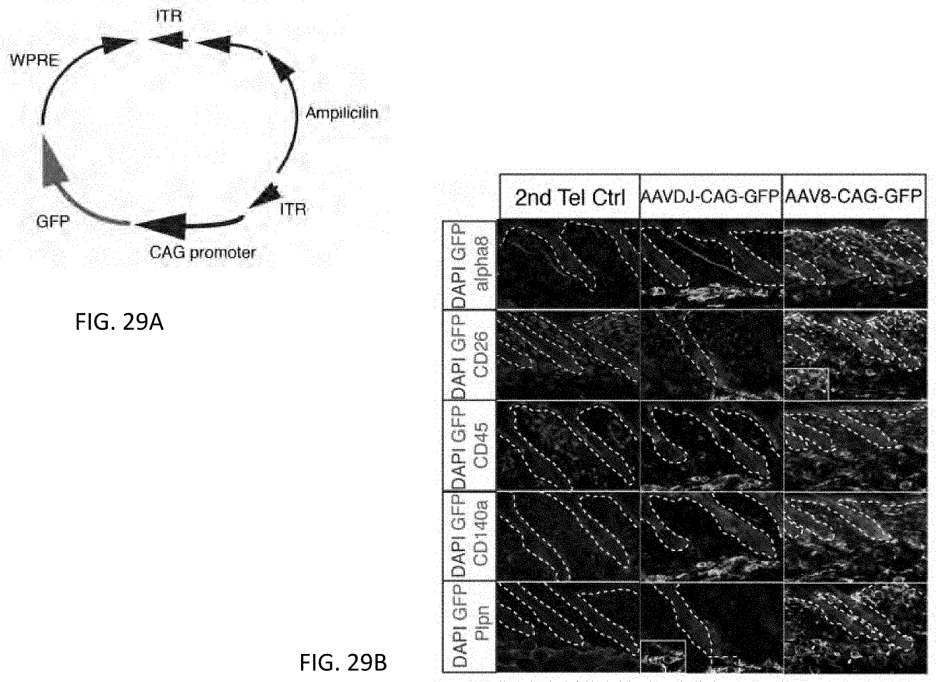

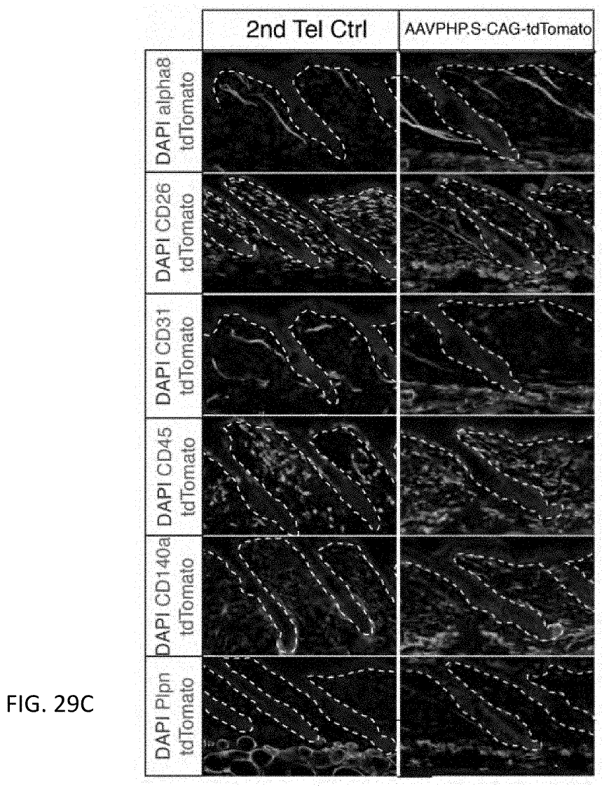

The skin is composed of multiple cells from different lineages, each with its own distinct function. Hence, it is essential to establish the specific cellular transduction patterns of the multiple AAV serotypes. For this aim, different AAV serotypes, including AAV8, AAVDJ, and AAVPHP. S, which either carried the GFP or the TdTomato fluorescent reporters to indicate infectivity, were dermally injected into mice during telogen, in which there is little proliferative activity occurring in the skin (FIG. 29A). After collecting the skin six days after injection and performing immunofluorescence staining, GFP and tdTomato expression varied between the different AAV serotypes. The AAV8 serotype showed an affinity for dermal fibroblasts (as indicated by the CD140a marker), adipocytes (Plpn marker), and the dermal papillae (Pcad marker, which marks the hair germ located right above the DP) while the AAVDJ serotype mainly transduced adipocytes (FIG. 29B). Furthermore, the AAVPHP. S serotype displayed a tendency to infect the arrector pili muscle (a8 marker) as well as dermal fibroblasts and adipocytes (FIG. 29C). This data suggests the AAV toolkit can be used in the skin as many serotypes could effectively transduce into different cell types of interest in the skin.

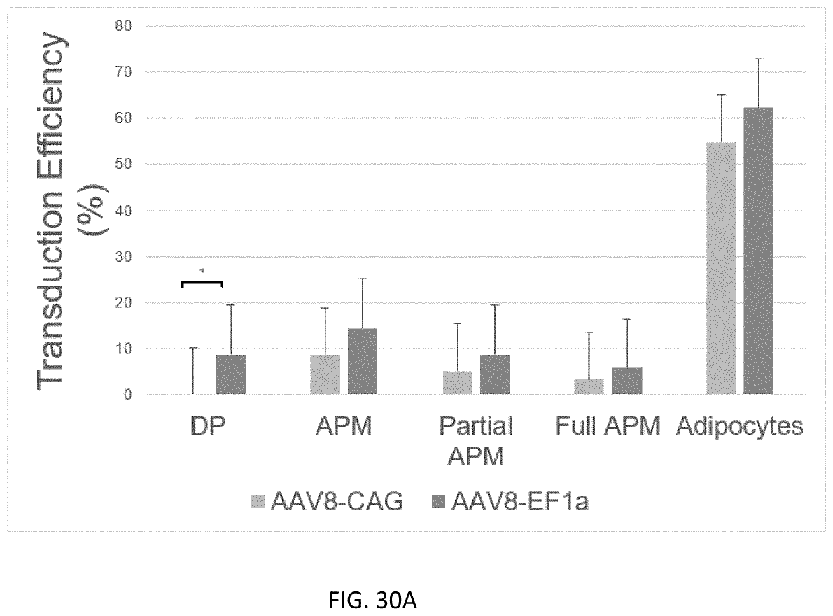

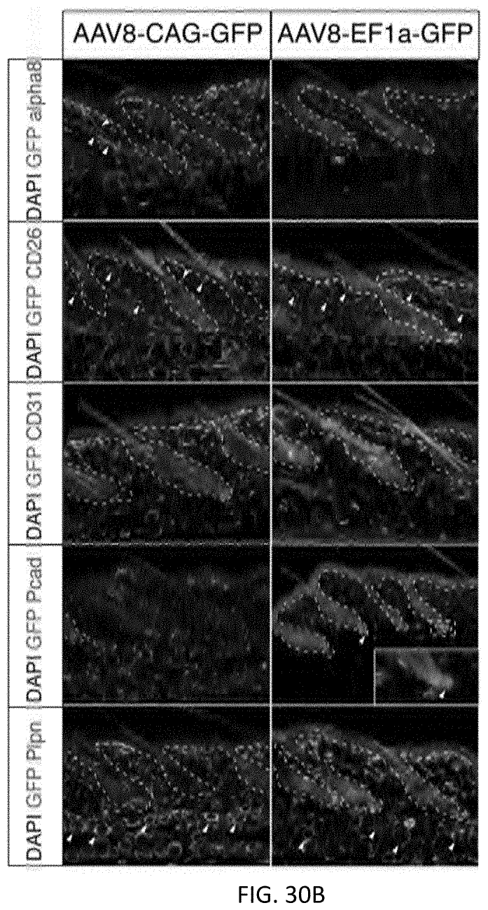

AAV8 serotype's broad infectivity patterns would be pivotal to establishing AAV as a useful transgene overexpression tool, because using AAV8 would ensure ectopic overexpression in many dermal cells. Since the transduction pattern can also depend on the promoter used to drive expression, the infectivity pattern of two ubiquitous promoters, namely the CAG and EF1a promoters, were checked. Interestingly, slight variations in infection targets and abundance were identified (FIG. 30). When the GFP expression was driven by the EF1a promoter, the AAV would infect the DP at a higher frequency (FIG. 30). In regard to other quantifiable cell types in the skin, including the APM and adipocytes, there was no significant difference between the two promoters. Qualitatively, the GFP signal created by AAV8-CAG-GFP was more intense than the GFP+ APMs infected by AAV8-EF1a. This data suggests that there are no significant differences in most transduction targets except for a higher infection of the DP by the AAV8-CAG virus.

### Validating AAVs as a Gene Delivery System to Uncover Edn3's Role in Maintaining Melanocyte Activation

After exploring the efficacy of AAVs in the skin, potential applications facilitated through delivering AAVs were examined. As a proof of concept, Shh, a known factor that facilitates anagen progression by inducing HFSC proliferation, was chosen. AAV8-Shh was delivered through intradermal injection into the skin of a mouse in the extended second telogen phase, when HFSCs are quiescent. Compared to control mice, AAV-Shh injected mice exhibited drastic darkening of the skin around the injection sites, an indicator of anagen entry. The mouse's early anagen entry suggests that the delivery of Shh to dermal cells activated HFSC proliferation. Thus, this data validates the use of AAVs as a gene delivery tool under normal conditions (FIG. 31A).

After demonstrating how AAV-mediated dermally overexpressed Shh can affect the hair follicles, whether AAV-mediated manipulation is limited to the epithelial compartment was examined by looking at the melanocyte population that resides inside the hair follicle. Endothelin-3 (Edn3) presented itself as an ideal candidate because of its previously established role in melanocyte development and maintenance during injury (Saldana-Caboverde and Kos 2010; Li et al. 2017). To explore the effects of Edn3 on the adult skin's melanocyte population, AAV-Edn3 during second telogen was intradermally delivered. To confirm appropriate delivery of the gene to cells in the skin, staining was performed for the Myc tag, which was included in the vector for this particular AAV (FIG. 31D). Interestingly, the Edn3-overexpressed mouse exhibited abnormal pigmentation in its ears 33 days after AAV delivery (FIG. 31B). In the Edn3-overexpressed skin, there was both ectopic pigmentation and heavy pigmentation inside the hair bulge region (FIG. 31C). These data suggest a novel role for Edn3 in regulating melanocyte activation from the dermis.

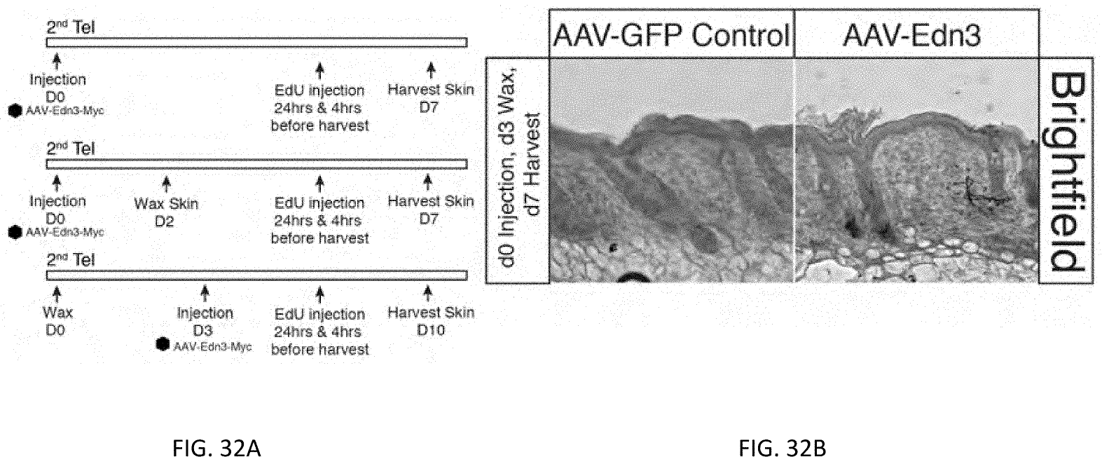

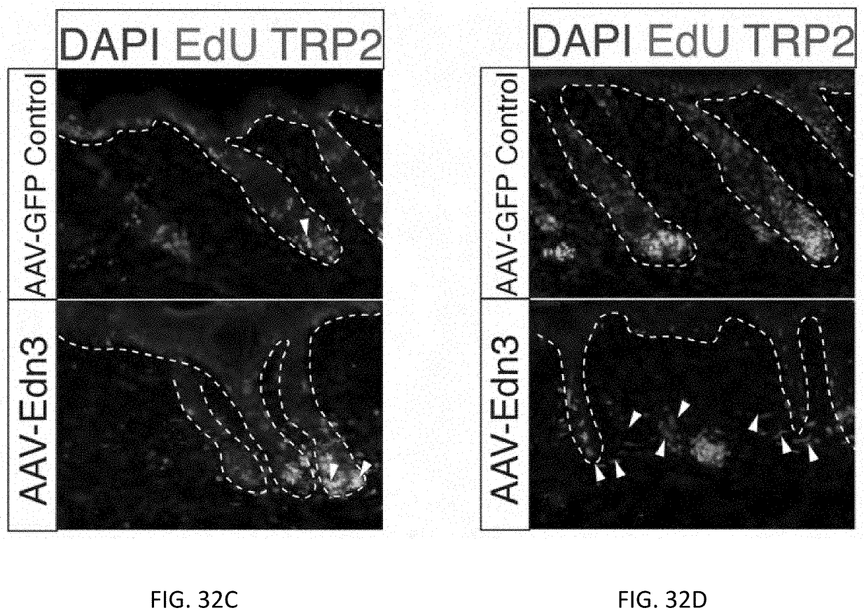

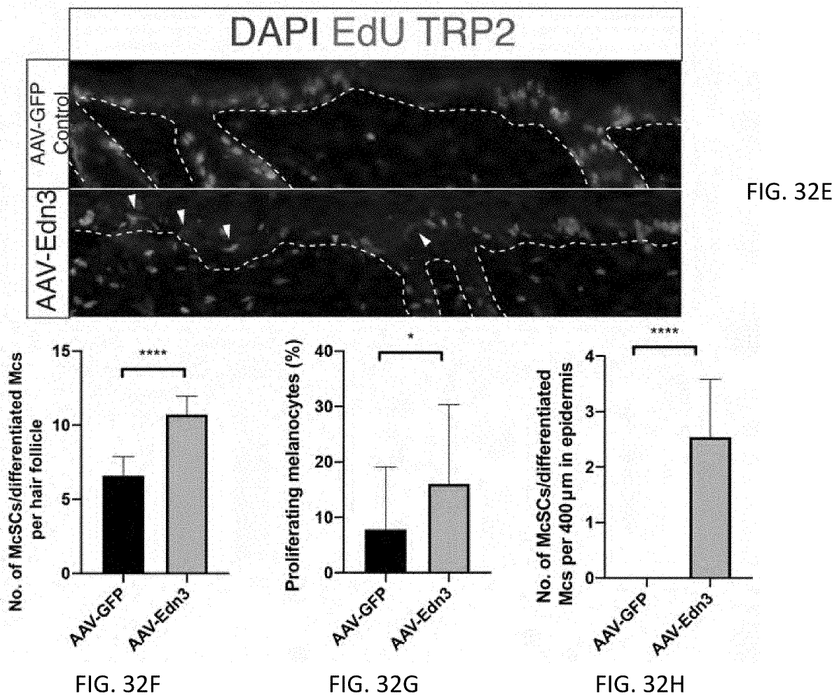

As Edn3 has a known role in some aspects of melanocyte development and maintenance during injury, its effects on the melanocyte population when it is overexpressed in the skin under normal conditions were assessed. McSCs normally reside in the bulge with HFSCs, and both populations are coordinately activated at the onset of anagen to generate a pigmented hair shaft (Nishimura et al. 2002). To observe how the hair cycle affects Edn3's effect on McSCs three conditions were observed and analyzed at an earlier time point: injection and harvest of skin in second telogen, injection and harvest of skin during anagen, and injection of Edn3 during telogen with harvest occurring during early anagen (FIG. 32A). This analysis allowed for the capture of how the environments and dynamics during telogen, anagen, and the telogen to anagen transition impact Edn3's effect on MsSCs. EdU was used to capture any proliferative activity and immunostaining for melanocytes stem cells as well as differentiated melanocytes (TRP2 marker), and the McSC population was observed for any aberrant activity. In the two conditions of injection and harvest during the same phase of the hair cycle, there was no observable difference in proliferation for both MsSCs and non-MsSCs. However, when Edn3 was injected and the skin was allowed to undergo anagen entry before collection, the skin exhibited abnormal and ectopic pigmentation (FIG. 32B). When the skin was visually analyzed, there were more melanocytes throughout the hair follicle and even in the epidermis, suggesting increased migration of melanocytes in the AAV-Edn3 injected mice (FIGS. 32C-E). Additionally, the increase in total number of melanocytes may be due to an increase in proliferative activity of melanocytes (FIG. 32G). Overall, these data suggest that the overexpression of Edn3 works in concordance with the changing environment during the telogen to anagen transition to activate McSCs, which can then migrate either into the epidermis or even into the dermis (FIG. 32D).

### Effects of AAV-Mediated Overexpression of Shh on Wound Healing

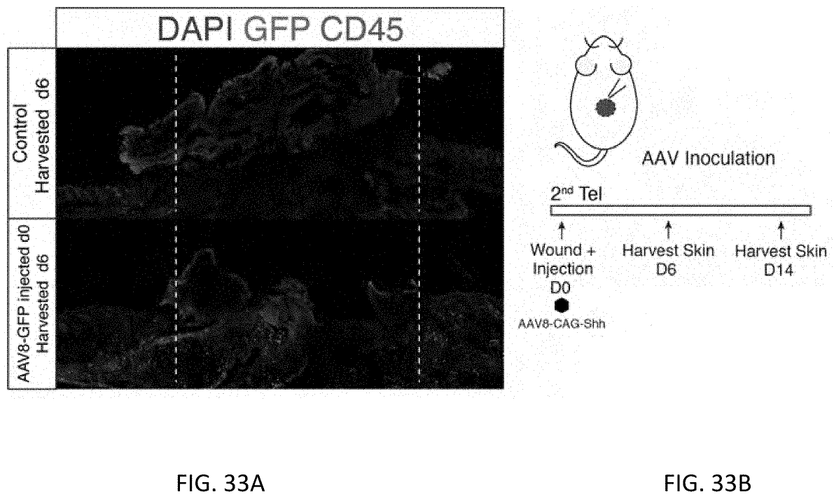

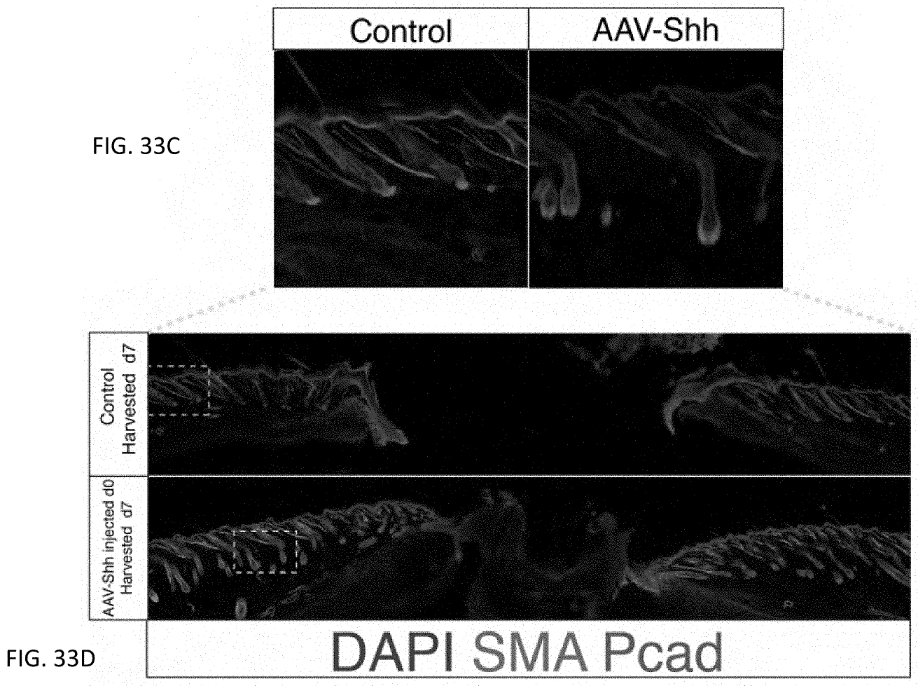

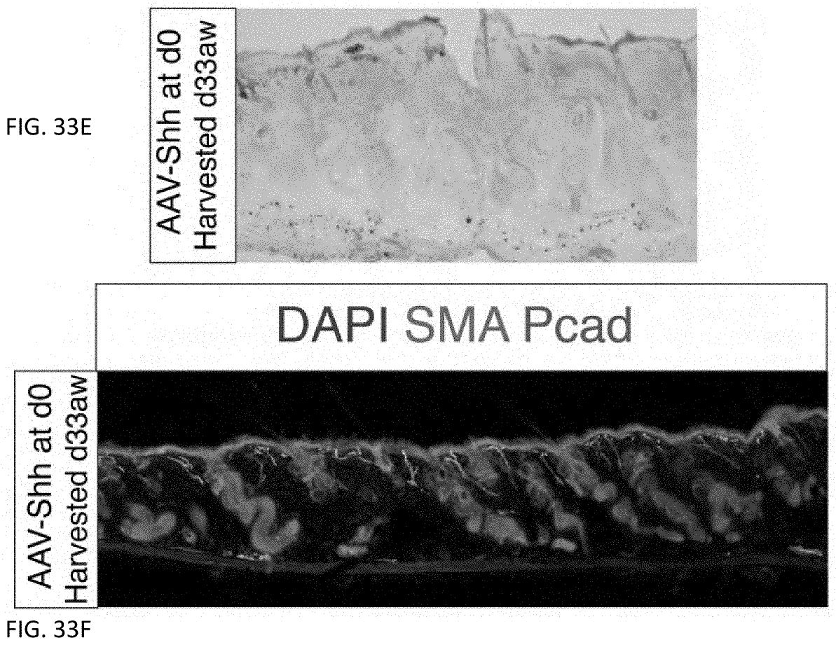

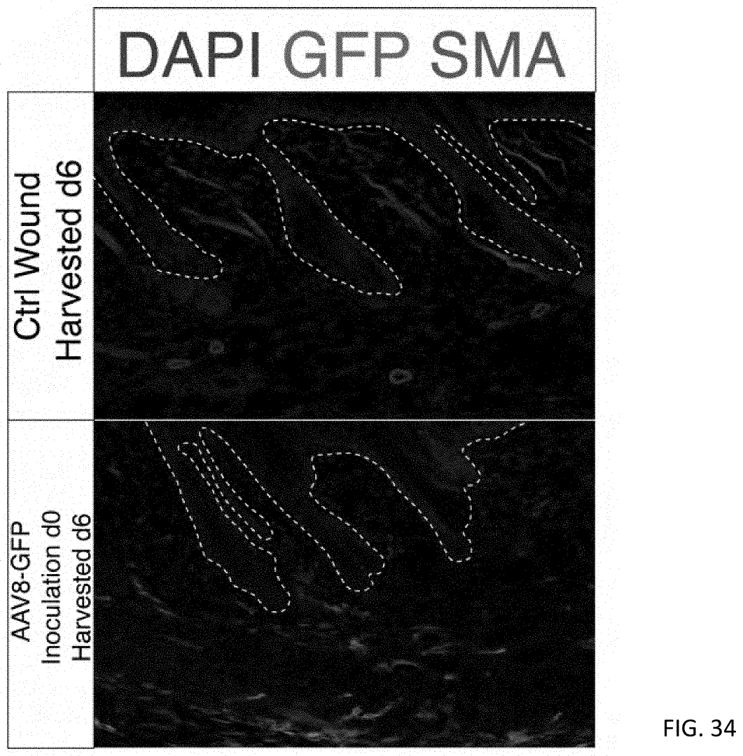

One of the most common assaults to the skin occurs during wounding, making wound healing critical to maintenance of homeostatic conditions. Moreover, the impairment of wound healing in pathological conditions, including diabetes, cardiovascular diseases, and autoimmune diseases, increases the need for possible enhancements to the process (Avishai, Yeghiazaryan, and Golubnitschaja 2017). In order to test the applicability of AAVs in the context of wound healing, the success of delivering AAVs into a wound was evaluated. After creating a small wound on the back of a mouse, a solution containing AAV8-GFP was pipetted onto the wound bed. Immunofluorescence staining was then conducted to detect GFP expression in the wounded skin. Interestingly, AAV8 transduced cells in this manner simply by dropping solution onto the wound (FIG. 33A). However, because there was no co-localization with immune cells or even myofibroblasts, two common cell types found in wounds, the cell type that the AAV infected was not identified (FIG. 33A; FIG. 34). Nevertheless, this data suggests that AAVs can be used to infect and manipulate cells in the dermis even under wounded conditions.

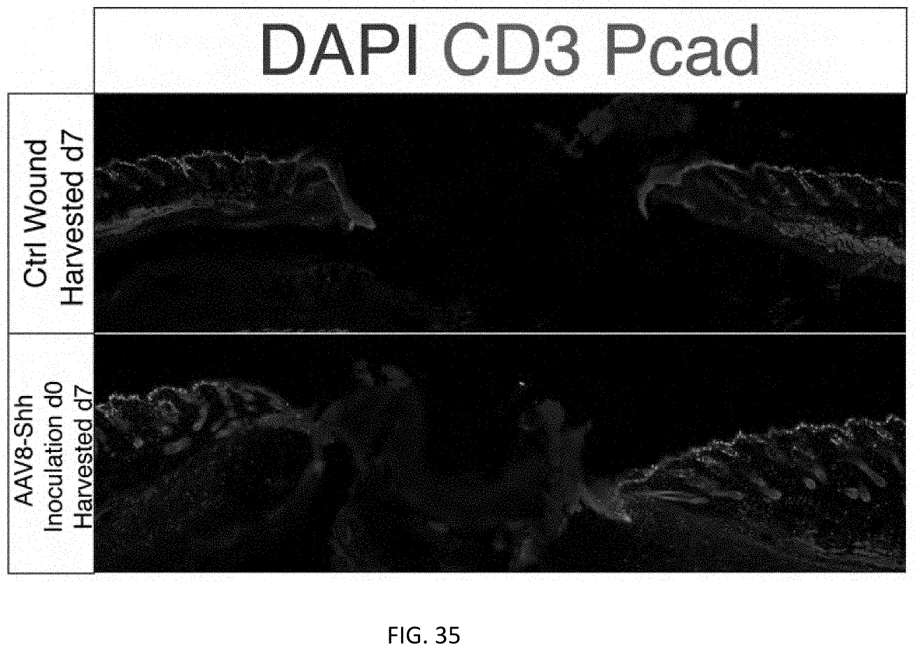

Following Lim et al. 's discovery that constitutive expression of dermal Shh can induce hair follicle neogenesis in healing wounds, whether it would be possible to induce alteration in the wound healing process by delivering AAV-Shh to the wound was examined (Lim et al. 2018). After solution containing AAV8-Shh was dropped onto the wounds of 3 mice, the healing wounded skin was collected at three different timepoints: 7 days, 14 days, and 33 days after wounding. In order to confirm that the AAV8-Shh induced overexpression of Shh in dermal cells, in-situ hybridization was performed to detect Shh mRNA expression in the wounded skin that was collected 33 days after wounding (FIG. 33E). Once ectopic Shh expression was confirmed in the dermis, the skin was analyzed for any differences during the healing process through immunofluorescence. The skin was stained for markers marking the epithelial layer (Pcad marker), immune cells (CD45 marker), myofibroblasts (SMA marker), and APM (SMA marker) to piece together a time-lapse of wound healing in both control and AAV-Shh infected mice (FIG. 33D; FIG. 35). In staining for Pcad, which marks the hair germ as well as the epithelial layer, the presence of hair bulbs deeper in the dermis layer, an indicator of anagen entry, confirmed ectopic Shh-induced HFSC proliferation (FIG. 33C). However, in comparing the AAV-Shh infected wounds to the control wounds, no significant difference in the rate of wound healing was found. The epithelial layer did not seem to re-epithelialize faster than control conditions, and there also seemed to be no difference in myofibroblasts, APM, or immune cell localization (FIGS. 33C-33D; FIG. 35). In the long-term regeneration and wound healing condition, there were both overgrown hair follicles and small hair follicles protruding near and from the epithelial layer (Pcad marker) (FIG. 33F). The overexpression of Shh most likely caused both the aberrant growth of certain hair follicles and hair follicle neogenesis. This finding is important because hair follicles do not normally regenerate after wounding in the adult skin. Besides causing hair follicle neogenesis in the long term, these data suggest that ectopic overexpression of Shh in the wound does not have a significant immediate effect on the process of wound healing.

### Discussion

The AAV toolkit has proved promising in a wide range of organ systems, including in muscles, the liver, and neurons, as a convenient and effective mode of genetic manipulation (Tabebordbar et al. 2016; Li et al. 2019; Haggerty et al. 2020). Here, it was demonstrated that AAVs can also similarly serve as a tool in the skin by addressing two main questions. Not only can AAVs infect a wide variety of cell types in the skin under normal conditions, allowing for the possibility of specific genetic manipulation, but they can also transduce cells in wounded conditions through a topical application. Additionally, it was shown that Edn3 activates McSC, providing an example of how AAVs can help identify a factor's role in maintaining a skin stem cell population.

As demonstrated through overexpression of Edn3 in the skin, the use of AAVs provides a convenient rapid tool to explore the function of secreted factors. Through AAV-mediated overexpression of Edn3, it was demonstrated that it has a novel role in regulating McSC activity from the dermis during the telogen to anagen transition (Garcia et al. 2008). In the telogen to anagen condition, the significant increase seen in total number of melanocytes, their proliferative activity, and migration after Edn3 overexpression support this conclusion.

In the AAV-Shh overexpressed mouse, hair follicle neogenesis at the wound area 33 days after the initial wounding indicates that the overexpression of Shh can aid in remodeling tissue architecture. Even though it was unclear where the initial wound site was 33 days after, the aberrant growth of hair follicles and budding new hair follicles throughout the skin supports hair follicle neogenesis occurring in the wound. This phenotype supports both the effects of AAV-mediated overexpression of Shh on existing hair follicles and the possibility that Shh overexpression caused new hair follicles to grow in the wound bed.

Given all the possible routes one could take to potentially enhance wound healing, one interesting aspect of using AAVs is its inability to infect the epidermis. Although the wound provides a condition in which the epithelial layer is broken, inoculating AAVs into the wound bed did not lead to their infection of epidermal cells. One possible explanation is the epidermis acting as a physical barrier so that the AAVs can only enter into the dermis. However, there is still an obvious phenotypic effect that can occur through dermal overexpression.

### Materials and Methods

### Mice

All animals were maintained in an Association for Assessment and Accreditation of Laboratory Animal Care-approved animal facility at Harvard University, and procedures were performed with Institutional Animal Care and Use Committee-approved protocols.

### Hair Cycle Timing

Subdivisions of hair cycle into telogen and anagen stages were based on Muller-Rover et al. 2001. Since hair cycles vary among strains and sexes, stages instead of exact mouse ages were evaluated and carefully monitored for each experiment.

### AAV Generation and Administration

The following commercially available constructs were used: AAV8-CAG-GFP (BWH), AAVDJ-CAG-GFP (BWH), AAVPHP. S-CAG-tdTomato (Addgene), AAV8-EF1a-GFP (Vigene Biosciences), AAV8-CAG-Edn3 (Welgen), and AAV8-CAG-Shh (Welgen) viruses.

All AAV viruses were injected intradermally. Viral stock was diluted to a concentration of 1×1012 gc/mL with dPBS. 40 μl of the diluted virus was injected once intradermally. Dorsal skin was collected 6 to 33 days following injection, as indicated by results.

### Wounding and AAV

6 mm full thickness wounds were made onto the backs of wild type C57/B16 mice during second telogen using biopsy punches. 40 ul of the diluted AAV8-CAG-GFP or AAV8-CAG-Shh were added immediately after by pipetting onto the wounds.

### Waxing

Waxing of dorsal hair was achieved by repeatedly applying lukewarm wax, letting it dry, and then peeling the hair off.

### Histology and Immunohistochemistry

Dorsal skins were fixed for 15 minutes using 4% paraformaldehyde (PFA) at room temperature, washed with PBS, immersed in 30% sucrose overnight at 4° C., and embedded in OCT (Sakura Finetek). Forty μM sections were used for all staining unless otherwise noted. For all 40 μM thick immunofluorescent staining, slides were blocked (5% Donkey serum; 1% BSA, 2% Cold water fish gelatin in 0.3% Triton in PBS) for 1-4 hours at room temperature, incubated with primary antibody overnight at 4° C., then incubated with secondary antibody for 2-4 hours at room temperature or overnight at 4° C.

The following primary antibodies and dilutions were used: GFP (rabbit, Abcam ab290, 1:500), pCAD (goat, R&D AF761, 1:200), CD3 (rat, Thermo Fisher Scientific 14-0032-82, 1:100), CD140a (rat, eBioscience 14-1401-82, 1:100), CD140a (goat, R&D AF1062-SP, 1:100), CD26 (rat, R&D MAB954-SP, 1:100), Perilipin A (goat, Abcam ab61682, 1:400), CD31 (rat, BD 550274, 1:100), alpha8 (goat, R&D AF4076-SP, 1:100), SMA (rabbit, Abcam ab5694, 1:400), GFP_FITC (goat, Abcam ab6662, 1:500), Myc (rabbit, Cell Signaling 2278, 1:100). The following secondary antibodies were used: donkey anti-rabbit conjugated with Alexa 488 or Alexa 549 (Jackson ImmunoResearch 711-545-152 and 711-165-152, 1:250), donkey anti-rat conjugated with Alexa 488 or Alexa 549 (Jackson ImmunoResearch 712-545-150 and 712-165-153, 1:250), donkey anti-goat conjugated with Alexa 647 (Jackson ImmunoResearch 705-605-147, 1:250). Samples were mounted in Prolong Gold with DAPI (Life Technologies).

For immunofluorescence staining using melanocyte markers Trp2 and Tyrp1, 40 μM slides underwent methanol fixation in 0.3% H₂O₂ in methanol after PFA fixation and then followed immunofluorescence protocol. The following antibodies and dilutions were used: Tyrp1 (rabbit, Ting Cheng lab, 1:400) and Trp2 (goat, Santa Cruz Biotechnology sc-10451, 1:500).

### EdU Injection and Immunohistochemistry

For each gram a mouse weighed, 5 μl of 5 mg/ml EdU was injected intraperitoneally 24 hours and 4 hours before dorsal skin harvest. Then after incubation with primary antibody according to immunohistochemistry protocol, 40 μM thick slides were incubated with EdU staining cocktail (10× Reaction Buffer Component D, sterile milliQ H₂O, CuSO₄, Alexa Fluor Azide, Diluted Reaction Buffer Additive Component F 10×) for 40 minutes before proceeding with incubation of secondary antibodies according to immunohistochemistry protocol.

In situ Hybridization

The in situ hybridization was performed using the RNAscope 2.5 HD Detection Kit-RED according to manufacturer's instructions (Cat. No. 322360). 14 μM thick slides were first incubated in 50% EtOH, then 70% EtOH, then 100% EtOH, and left in 100% EtOH overnight. Slides were then pretreated and then incubated with Shh-specific RNA probe (RNAscope Probe-Mm-Shh Cat. No. 314361). The slides are then treated with a series of signal amplification molecules and then incubated with Fast Red substrate.

### Confocal Microscopy and Image Processing

Images were acquired with a Zeiss LSM 880 +FLIM microscope (Carl Zeiss Microlmaging) through a 40× oil objective or a 20× objective. Representative single Z planes are presented and colocalizations were interpreted only in single Z stacks. Z stacks were projected using ImageJ software. RGB images were assembled in Adobe Photoshop and panels were labeled in Adobe Illustrator. Statistical analyses were performed in Excel and Prism GraphPad.

### REFERENCES

Avishai, E., K. Yeghiazaryan, and O. Golubnitschaja. 2017. ‘Impaired wound healing: facts and hypotheses for multi-professional considerations in predictive, preventive and personalised medicine’, EPMA J, 8: 23-33.

Driskell, R. R., B. M. Lichtenberger, E. Hoste, K. Kretzschmar, B. D. Simons, M. Charalambous, S. R. Ferron, Y. Herault, G. Pavlovic, A. C. Ferguson-Smith, and F. M. Watt. 2013. ‘Distinct fibroblast lineages determine dermal architecture in skin development and repair’, Nature, 504: 277-81.

Gaj, T., B. E. Epstein, and D. V. Schaffer. 2016. ‘Genome Engineering Using Adeno-associated Virus: Basic and Clinical Research Applications’, Mol Ther, 24: 458-64.

Garcia, R. J., A. Ittah, S. Mirabal, J. Figueroa, L. Lopez, A. B. Glick, and L. Kos. 2008. ‘Endothelin 3 induces skin pigmentation in a keratin-driven inducible mouse model’, J Invest Dermatol, 128: 131-42.

Gurtner, G. C., S. Werner, Y. Barrandon, and M. T. Longaker. 2008. ‘Wound repair and regeneration’, Nature, 453: 314-21.

Haggerty, D. L., G. G. Grecco, K. C. Reeves, and B. Atwood. 2020. ‘Adeno-Associated Viral Vectors in Neuroscience Research’, Mol Ther Methods Clin Dev, 17: 69-82.

Hirsch, T., T. Rothoeft, N. Teig, J. W. Bauer, G. Pellegrini, L. De Rosa, D. Scaglione, J. Reichelt, A. Klausegger, D. Kneisz, 0. Romano, A. Secone Seconetti, R. Contin, E. Enzo, I. Jurman, S. Carulli, F. Jacobsen, T. Luecke, M. Lehnhardt, M. Fischer, M. Kueckelhaus, D. Quaglino, M. Morgante, S. Bicciato, S. Bondanza, and M. De Luca. 2017. ‘Regeneration of the entire human epidermis using transgenic stem cells’, Nature, 551: 327-32.

Hsu, Y. C., L. Li, and E. Fuchs. 2014a. ‘Emerging interactions between skin stem cells and their niches’, Nat Med, 20: 847-56.

Hsu, Y. C., L. Li, and E. Fuchs. 2014b. ‘Transit-amplifying cells orchestrate stem cell activity and tissue regeneration’, Cell, 157: 935-49.

Hsu, Y. C., H. A. Pasolli, and E. Fuchs. 2011. ‘Dynamics between stem cells, niche, and progeny in the hair follicle’, Cell, 144: 92-105.

Ito, M., Y. Liu, Z. Yang, J. Nguyen, F. Liang, R. J. Morris, and G. Cotsarelis. 2005. ‘Stem cells in the hair follicle bulge contribute to wound repair but not to homeostasis of the epidermis’, Nat Med, 11: 1351-4.

Ito, M., Z. Yang, T. Andl, C. Cui, N. Kim, S. E. Millar, and G. Cotsarelis. 2007. ‘Wnt-dependent de novo hair follicle regeneration in adult mouse skin after wounding’, Nature, 447: 316-20.

Kos, S., T. Blagus, M. Cemazar, U. Lampreht Tratar, M. Stimac, L. Prosen, T. Dolinsek, U. Kamensek, S. Kranjc, L. Steinstraesser, G. Vandermeulen, V. Preat, and G. Sersa. 2016. ‘Electrotransfer parameters as a tool for controlled and targeted gene expression in skin’, Mol Ther Nucleic Acids, 5: e356.

Li, A., C. M. Lee, A. E. Hurley, K. E. Jarrett, M. De Giorgi, W. Lu, K. S. Balderrama, A. M. Doerfler, H. Deshmukh, A. Ray, G. Bao, and W. R. Lagor. 2019. ‘A Self-Deleting AAV-CRISPR System for In Vivo Genome Editing’, Mol Ther Methods Clin Dev, 12: 111-22.

Li, H., L. Fan, S. Zhu, M. K. Shin, F. Lu, J. Qu, and L. Hou. 2017. ‘Epilation induces hair and skin pigmentation through an EDN3/EDNRB-dependent regenerative response of melanocyte stem cells’, Sci Rep, 7: 7272.

Lim, C. H., Q. Sun, K. Ratti, S. H. Lee, Y. Zheng, M. Takeo, W. Lee, P. Rabbani, M. V. Plikus, J. E. Cain, D. H. Wang, D. N. Watkins, S. Millar, M. M. Taketo, P. Myung, G. Cotsarelis, and M. Ito. 2018. ‘Hedgehog stimulates hair follicle neogenesis by creating inductive dermis during murine skin wound healing’, Nat Commun, 9: 4903.

Muller-Rover, S., B. Handjiski, C. van der Veen, S. Eichmuller, K. Foitzik, I. A. McKay, K. S. Stenn, and R. Paus. 2001. ‘A comprehensive guide for the accurate classification of murine hair follicles in distinct hair cycle stages’, J Invest Dermatol, 117: 3-15.

Nishimura, E. K., S. A. Jordan, H. Oshima, H. Yoshida, M. Osawa, M. Moriyama, I. J. Jackson, Y. Barrandon, Y. Miyachi, and S. Nishikawa. 2002. ‘Dominant role of the niche in melanocyte stem-cell fate determination’, Nature, 416: 854-60.

Platt, R. J., S. Chen, Y. Zhou, M. J. Yim, L. Swiech, H. R. Kempton, J. E. Dahlman, O. Parnas, T. M. Eisenhaure, M. Jovanovic, D. B. Graham, S. Jhunjhunwala, M. Heidenreich, R. J. Xavier, R. Langer, D. G. Anderson, N. Hacohen, A. Regev, G. Feng, P. A. Sharp, and F. Zhang. 2014. ‘CRISPR-Cas9 knockin mice for genome editing and cancer modeling’, Cell, 159: 440-55.

Plikus, M. V., J. A. Mayer, D. de la Cruz, R. E. Baker, P. K. Maini, R. Maxson, and C. M. Chuong. 2008. ‘Cyclic dermal BMP signalling regulates stem cell activation during hair regeneration’, Nature, 451: 340-4.

Ran, F. A., L. Cong, W. X. Yan, D. A. Scott, J. S. Gootenberg, A. J. Kriz, B. Zetsche, O. Shalem, X. Wu, K. S. Makarova, E. V. Koonin, P. A. Sharp, and F. Zhang. 2015. ‘In vivo genome editing using Staphylococcus aureus Cas9’, Nature, 520: 186-91.

Saldana-Caboverde, A., and L. Kos. 2010. ‘Roles of endothelin signaling in melanocyte development and melanoma’, Pigment Cell Melanoma Res, 23: 160-70.

Srivastava, A. 2016. ‘In vivo tissue-tropism of adeno-associated viral vectors’, Curr Opin Virol, 21: 75-80.

Tabebordbar, M., K. Zhu, J. K. W. Cheng, W. L. Chew, J. J. Widrick, W. X. Yan, C. Maesner, E. Y. Wu, R. Xiao, F. A. Ran, L. Cong, F. Zhang, L. H. Vandenberghe, G. M. Church, and A. J. Wagers. 2016. ‘In vivo gene editing in dystrophic mouse muscle and muscle stem cells’, Science, 351: 407-11.

Woo, W. M., H. H. Zhen, and A. E. Oro. 2012. ‘Shh maintains dermal papilla identity and hair morphogenesis via a Noggin-Shh regulatory loop’, Genes Dev, 26: 1235-46.

Wu, Z., A. Asokan, and R. J. Samulski. 2006. ‘Adeno-associated virus serotypes: vector toolkit for human gene therapy’, Mol Ther, 14: 316-27.

What is claimed is:

1. A delivery system comprising an adeno-associated virus (AAV) and a promoter for delivery of a gene to a cell selected from the group consisting of fibroblasts, dermal papilla, adipocytes, arrector pili muscle, sensory nerves, sympathetic nerves, immune cells, and panniculus carnosus.

2. The delivery system of claim 1, wherein the promoter is selected from the group consisting of CAG, EF1a, NPY, and hSYN.

3. The delivery system of claim 1 or claim 2, wherein the AAV is selected from the group consisting of AAV2, AAV6, AAV8, AAV9, AAVrh10, AAV-DJ, AAV-PHP. S, and AAV-retro.

4. The delivery system of any one of claims 1-3, wherein the AAV is selected from the group consisting of AAV8, AAVrh10, AAV6, AAV-PHP. S, and AAV-retro.

5. The delivery system of any one of claims 1-3, wherein the AAV comprises AAV2, the promoter comprises CAG, and the cell comprises adipocytes.

6. The delivery system of any one of claims 1-3, wherein the AAV comprises AAV9, the promoter comprises CAG, and the cell is selected from the group consisting of adipocytes, fibroblasts, and arrector pili muscle.

7. The delivery system of any one of claims 1-3, wherein the AAV comprises AAV-DJ, the promoter comprises CAG, and the cell comprises adipocytes.

8. The delivery system of any one of claims 1-4, wherein the AAV comprises AAV8, the promoter comprises CAG, and the cell is selected from the group consisting of fibroblasts, dermal papilla, adipocytes, arrector pili muscle, and immune cells.

9. The delivery system of any one of claims 1-4, wherein the AAV comprises AAV8, the promoter comprises EF1a, and the cell is selected from the group consisting of fibroblasts, dermal papilla, adipocytes, arrector pili muscle, and immune cells.

10. The delivery system of any one of claims 1-4, wherein the AAV comprises AAVrh10, the promoter comprises CAG, and the cell is selected from the group consisting of fibroblasts, adipocytes, and arrector pili muscle.

11. The delivery system of any one of claims 1-4, wherein the AAV comprises AAV6, the promoter comprises CAG, and the cell is selected from the group consisting of fibroblasts, adipocytes, and arrector pili muscle.

12. The delivery system of any one of claims 1-4, wherein the AAV comprises AAV6, the promoter comprises EF1a, and the cell comprises adipocytes and arrector pili muscle.

13. The delivery system of any one of claims 1-4, wherein the AAV comprises AAV-PHP. S, the promoter comprises CAG, and the cell is selected from the group consisting of fibroblasts, adipocytes, arrector pili muscle, sensory nerves, sympathetic nerves and panniculus carnosus.

14. The delivery system of any one of claims 1-4, wherein the AAV comprises AAV-PHP. S, the promoter comprises EF1a, and the cell is selected from the group consisting of fibroblasts, dermal papilla, adipocytes, and arrector pili muscle.

15. The delivery system of any one of claims 1-4, wherein the AAV comprises AAV-PHP. S, the promoter comprises NPY, and the cell is selected from the group consisting of sensory nerves and sympathetic nerves.

16. The delivery system of any one of claims 1-4, wherein the AAV comprises AAV-PHP. S, the promoter comprises hSYN, and the cell is selected from the group consisting of sensory nerves and sympathetic nerves.

17. The delivery system of any one of claims 1-4, wherein the AAV comprises AAV-retro, the promoter comprises CAG, and the cell is selected from the group consisting of adipocytes and sympathetic nerves.

18. The delivery system of any one of claims 1-4, wherein the AAV comprises AAV-retro, the promoter comprises hSYN, and the cell comprises sympathetic nerves.

19. A delivery system comprising an adeno-associated virus (AAV) and a promoter for delivery of a gene to an arrector pili muscle (APM) or a fibroblast.

20. The delivery system of claim 19, wherein the AAV is AAV-PHP. S.

21. The delivery system of claim 19, wherein the promoter is CAG.

22. A delivery system comprising an adeno-associated virus (AAV) and a promoter for delivery of a gene to a skin cell, wherein the AAV is AAV-PHP. S, wherein the enhancer is CAG, and wherein the skin cell is not a sympathetic nerve, a blood vessel, or a dermal sheath.

23. The delivery system of any of claims 19-22, wherein the gene is a DTA.

24. A delivery system comprising an adeno-associated virus (AAV) and a promoter for delivery of a gene to a hair follicle stem cell (HFSC).

25. The delivery system of claim 24, wherein the AAV is AAV8.

26. The delivery system of claim 24, wherein the promoter is CAG.

27. The delivery system of claim 24, wherein the gene is FGF18.

28. The delivery system of any one of claims 1-27, wherein the delivery system is suitable for administration to a patient via intradermal injection.

29. A pharmaceutical composition comprising the delivery system of any one of claims 1-28.

30. A method of treating a condition, disease, or disorder in a subject comprising administering the pharmaceutical composition of claim 29 to the subject.

31. A method of encouraging hair growth in a subject comprising elevating sympathetic nerve activity by exposing the subject to a cold temperature for a period of at least two hours.

32. The method of claim 31, wherein the exposure to the cold temperature activates hair follicle stem cells (HFSCs).

33. The method of claim 31, wherein the exposure to the cold temperature results in enhanced c-Fos expression.

34. The method of claim 31, wherein the cold temperature is a temperature of about 5° C.

35. The method of any of claims 31-34, wherein the cold temperature is applied directly and/or specifically to the location of desired hair growth.

36. The method of claim 35, wherein the location of desired hair growth is the scalp.


SEQUENCE LISTING (16 sequences)

SEQ ID NO: 1 - DNA, 21 bp, Artificial
  primer
tggggccagtcacatccttat

SEQ ID NO: 2 - DNA, 21 bp, Artificial
  primer
tgacgcacaacacatcaatgg

SEQ ID NO: 3 - DNA, 20 bp, Artificial
  primer
tacacaggggagccaaacac

SEQ ID NO: 4 - DNA, 20 bp, Artificial
  primer
tcacaaagccttccatgcct

SEQ ID NO: 5 - DNA, 20 bp, Artificial
  primer
cctgtatgcctctggtcgta

SEQ ID NO: 6 - DNA, 20 bp, Artificial
  primer
ccatctcctgctcgaagtct

SEQ ID NO: 7 - DNA, 18 bp, Artificial
  primer
gtcttgtggcgttctgca

SEQ ID NO: 8 - DNA, 18 bp, Artificial
  primer
gctggacccgttctggat

SEQ ID NO: 9 - DNA, 20 bp, Artificial
  primer
cccaggacttgaatgtgctt

SEQ ID NO: 10 - DNA, 20 bp, Artificial
  primer
actgctgtgcttccaggttc

SEQ ID NO: 11 - DNA, 20 bp, Artificial
  primer
gggaccgcagcaagtacggc

SEQ ID NO: 12 - DNA, 20 bp, Artificial
  primer
cggatttggccgccacggag

SEQ ID NO: 13 - DNA, 19 bp, Artificial
  primer
ggtaacaagggggtcggag

SEQ ID NO: 14 - DNA, 20 bp, Artificial
  primer
ttgcaacgctctgtcatcag

SEQ ID NO: 15 - DNA, 21 bp, Artificial
  primer
ctcttgcctacaagcagttca

SEQ ID NO: 16 - DNA, 19 bp, Artificial
  primer
ccgtgttctcctcgtcctt